(19) United States
(12) Patent Application Publication
(10) Pub. No.: US 2009/0186343 A1
(43) Pub. Date: Jul. 23, 2009

(54) METHODS FOR PREPARING MODIFIED BIOMOLECULES, MODIFIED BIOMOLECULES AND METHODS FOR USING SAME

(75) Inventors: Hongyi Wang, Houston, TX (US); Norha Deluge, Pearland, TX (US); Kristi Kincaid, Houston, TX (US); Yuri Belosludtsev, The Woodlands, TX (US); Tommie Lincecum, JR., Houston, TX (US); Amy Williams, Houston, TX (US); Amy Bryant, League City, TX (US); Ming Fa, Pearland, TX (US); Benjamin Stevens, Houston, TX (US); Susan H. Hardin, College Station, TX (US)

Correspondence Address: INVITROGEN CORPORATION;C/O INTELLEVATE, P.O. BOX 52050, MINNEAPOLIS, MN, 55402, US

(73) Assignee: VISIGEN BIOTECHNOLOGIES, INC., Houston, TX (US)

(21) Appl. No.: 12/035,352

(22) Filed: Feb. 21, 2008

Related U.S. Application Data
(60) Provisional application No. 60/891,029, filed on Feb. 21, 2007.

(30) Foreign Application Priority Data
Jan. 28, 2003 (JP) EP 03002027.5

(51) Int. Cl. C12Q 1/68; C12P 19/34

(52) U.S. Cl. 435/6; 536/124

(57) ABSTRACT

A novel and efficient single pot synthetic schemes are disclosed for preparing modified nucleotides, nucleotide analogs, nucleotide polyphosphates, and nucleotide polyphosphate analogs. The novel method is used to prepare nucleotides, nucleotide analogs, nucleotide polyphosphates, and nucleotide polyphosphate analogs having non-persistent or persistent and non-persistent modifications. Novel biomolecular reactions are also disclosed using the novel modified biomolecules disclosed herein.

### RELATED APPLICATIONS

This application claims priority to and the benefit of U.S. Provisional Patent Application Ser. No. 60/891,029 filed Feb. 21, 2007, incorporated herein by reference. The present application also is related to the following U.S. patent application Ser. Nos. 11/007,642, filed Dec. 8, 2004; 11/007,797, filed Dec. 8, 2004, now U.S. Pat. No. 7,329,492, issued Feb. 12, 2008; 11/648,164, filed Dec. 29, 2006, 11/648,722, filed Dec. 29, 2006; 11/648,174, filed Dec. 29, 2006; 11/648,191, filed Dec. 29, 2006; 11/648,136, filed Dec. 29, 2006; 11/648,182, filed Dec. 29, 2006; 11/648,713, filed Dec. 29, 2006; 11/648,106, filed Dec. 29, 2006; 11/648,115, filed Dec. 29, 2006; 11/648,137, filed Dec. 29, 2006; 11/648,856, filed Dec. 29, 2006; 11/648,184, filed Dec. 29, 2006; 11/648,138, filed Dec. 29, 2006; PCT/US01/21811, filed Jul. 9, 2001; 60/216,594, filed Jul. 7, 2000; 09/901,782, filed Jul. 9, 2001; 11/648,107, filed Dec. 29, 2006; 11/648,721, filed Dec. 29, 2006; 11/648,114, filed Dec. 29, 2006; 11/648,108, filed Dec. 29, 2006; 11/648,723, filed Dec. 29, 2006; PCT/US01/45819, filed Dec. 3, 2001; 60/250,764, filed Dec. 1, 2000; 10/007,621, filed Dec. 3, 2001, now U.S. Pat. No. 7,211,414, issued May 1, 2007; 60/527,909, filed Dec. 8, 2003; 11/007,794, filed Dec. 8, 2004; 60/832,010, filed Jul. 20, 2006; 11/781,157, filed Jul. 20, 2007; 60/765,693, filed Feb. 6, 2006; 11/671,956, filed Feb. 6, 2007; 60/787,434, filed Mar. 30, 2006; 11/694,605, filed Mar. 30, 2007; 60/832,097, filed Jul. 20, 2006; 11/781,160, filed Jul. 20, 2007; 60/832,098, filed Jul. 20, 2006; 11/781,166, filed Jul. 20, 2007, incorporated herein by reference.

### GOVERNMENTAL INTEREST

Governmental entities may have certain rights in and to the contents of this application due to funding from NIH NHGRI grant 5R01HG003580.

### BACKGROUND OF THE INVENTION

1. Field of the Invention

The present invention relates to modified biomolecules including one or a plurality of modifying groups, to methods for preparing modified biomolecules, especially modified nucleotides, and to methods for using same.

More particularly, the present invention relates to modified biomolecules including one or a plurality of modifying groups. The present invention also relates to methods for preparing modified biomolecules, especially nucleotides, including the step contacting a mono or polyphosphate biomolecule, especially mono or polyphosphate nucleotides, with a group having a detectable property to form a modified biomolecule having the group bonded to one of its phosphate moieties. Alternatively, the mono or polyphosphate biomolecule can be contacted with a linker including a leaving group to form a biomolecule bearing the linker bonded to one of its phosphate groups. The method can also include the step of attaching a group having a detectable property to the linker to form a biomolecule bearing the group bonded to one of its phosphate moieties through the linker. The present invention also includes methods for using same.

2. Description of the Related Art

Many synthetic procedures have been developed to prepare modified nucleotides. However, a large majority of these synthetic procedures are directed to preparing nucleotides that bear modifying moieties on the part of the structure that remains associated with the nucleotide after the nucleotide undergoes an incorporation reaction using a nucleotide polymerizing agent.

However, recent interest in producing native DNA strands (i.e., single molecule sequencing and enzyme activity assays) demonstrates that there is a need in the art for efficient methods that prepare phosphate modified nucleotides, especially structures that have multiple nucleotides bonded to the structure through one of the nucleotide's phosphate moieties.

### DEFINITIONS OF THE INVENTION

A nucleoside is a molecule including a sugar, usually ribose or deoxyribose, and a purine or pyrimidine base (sometimes referred to as a natural nucleoside).

A nucleoside analog is a nucleoside that includes a chemical modification to a portion of the nucleoside structure such as the natural or synthetic base, and/or the natural or synthetic sugars, e.g., the base and/or sugar can include groups bonded to atoms making up the sugar and/or base, can include atomic substitutions in the sugar or base, or can include both atomic substitutions in the sugar or base and groups bonded to atoms making up the sugar and/or base.

A nucleotide is any nucleoside with at least one phosphate attached to a nucleoside or a nucleoside analog.

A nucleotide type is a nucleotide having a specific base, where the base is a naturally occurring or synthetic base that adds complementary to a template base. For naturally occurring bases, the bases are selected from the group consisting of A, T, C, G, U, or other naturally occurring bases that can add complementary to a template base.

A nucleotide analog is a nucleotide that includes a chemical modification to a portion of the nucleotide structure such as the natural or synthetic base, the natural or synthetic sugars, and/or the phosphates and/or synthetic phosphate replacement moieties or groups, e.g., the base, sugar and/or phosphates can include other groups bonded to atoms making up the sugar, base and/or phosphates, can include atomic substitutions in the sugar, base, and/or phosphates can include both atomic substitutions in the sugar, base and/or phosphates and groups bonded to atoms making up the sugar, base and/or phosphates.

A nucleotide monophosphate is a nucleoside combined with a single phosphate group and forming the basic constituent of DNA and RNA (sometimes referred to as a natural nucleotide).

A nucleotide monophosphate analog is a nucleotide that includes a chemical modification to a portion of the nucleotide structure such as the natural or synthetic base, the natural or synthetic sugars, and/or the phosphates and/or synthetic phosphate replacement moieties or groups, e.g., the base, sugar and/or phosphates can include other groups bonded to atoms making up the sugar, base and/or phosphates, can include atomic substitutions in the sugar, base, and/or phosphates can include both atomic substitutions in the sugar, base and/or phosphates and groups bonded to atoms making up the sugar, base and/or phosphates.

A nucleotide polyphosphate is a nucleoside combined with more than one phosphate group and forming the basic monomers for polymerizing agents that form nucleic acids.

A nucleotide polyphosphate analog is a nucleotide that includes a chemical modification to a portion of the nucleotide structure such as the natural or synthetic base, the natural or synthetic sugars, and/or the phosphates or synthetic phosphate replacement moieties or groups.

A nucleotide triphosphate is a nucleoside combined with a triphosphate group.

A nucleotide triphosphate analog is a nucleotide triphosphate that includes a chemical modification to a portion of the nucleotide structure such as the natural or synthetic base, the natural or synthetic sugars, and/or the phosphates or synthetic phosphate replacement moieties or groups.

A nucleotide tetraphosphate is a nucleoside combined with a tetraphosphate group.

A nucleotide tetraphosphate analog is a nucleotide tetraphosphate that includes a chemical modification to a portion of the nucleotide structure such as the natural or synthetic base, the natural or synthetic sugars, and/or the phosphates or synthetic phosphate replacement moieties or groups.

A nucleotide pentaphosphate is a nucleoside combined with a pentaphosphate group.

A nucleotide pentaphosphate analog is a nucleotide pentaphosphate that includes a chemical modification to a portion of the nucleotide structure such as the natural or synthetic base, the natural or synthetic sugars, and/or the phosphates or synthetic phosphate replacement moieties or groups.

A nucleotide hexaphosphate is a nucleoside combined with a hexaphosphate group.

A nucleotide hexaphosphate analog is a nucleotide hexaphosphate that includes a chemical modification to a portion of the nucleotide structure such as the natural or synthetic base, the natural or synthetic sugars, and/or the phosphates or synthetic phosphate replacement moieties or groups, etc.

A persistently modified nucleoside, nucleotide, or nucleotide polyphosphate means a nucleoside, nucleotide or nucleotide polyphosphate analog where the modification remains with the nucleoside or nucleotide after undergoing a chemical or biochemical reaction such as incorporation into a growing nucleic acid sequence, i.e., the modification is on the base, the sugar and/or the backbone (alpha) phosphate.

A non-persistently modified nucleoside, nucleotide, or nucleotide polyphosphate means a nucleoside, nucleotide, or nucleotide polyphosphate analog where the modification is released after undergoing a chemical or biochemical reaction such as incorporation into a growing nucleic acid sequence, i.e., the modification is not on the base, the sugar and/or the backbone (alpha) phosphate.

A nucleotide polymerizing agent is an agent that is capable of polymerizing nucleotides in a stepwise fashion.

SAP means Shrimp Alkaline Phosphatase.

PDE1 means phosphodiesterase 1.

HPLC means High Pressure Liquid Chromatography.

TLC means Thin Layer Chromatography.

TEA means Triethylamine.

TEAB means Triethylamine bicarbonate.

DMSO means Dimethyl sulfoxide.

DMF means Dimethyl formamide.

AcN means Acetonitrile.

SAX means Strong Anion Exchange.

PEI-cellulose means Polyethyleneimine-cellulose.

NHS means N-hydroxysuccinimide.

Inc50 is a primer extension screening reaction in which the replicating complex (polymerase/primer/template) concentration is kept constant, while the concentration of dNTP is varied until 50% of the primer is extended. The Inc50 value is the concentration of nucleotide that supports 50% primer extension.

### SUMMARY OF THE INVENTION

### Methods of Modified Nucleotide Preparation

The present invention provides a method for preparing a modified biomolecule, where the method include the step of contacting a biomolecule including a phosphate group, a polyphosphate group or analog thereof and a modifying agent including a leaving group capable of being displaced by a phosphate group of the biomolecule under displacement reaction conditions to phosphate modified biomolecule.

The present invention provides a method for preparing a modified nucleotide including a non-persistent moiety, group or tag, including the step of contacting a natural nucleotide, nucleotide polyphosphate or an analog thereof and a modifying agent having a leaving group capable of being displaced by a phosphate of the natural nucleotide, nucleotide polyphosphate or the analog under displacement reaction conditions to form a phosphate modified nucleotide, phosphate modified polyphosphate or analog thereof.

The present invention also provides a method, system, apparatus and composition for sequencing nucleic acids including extending a template using a modified nucleotide of this invention, where the method comprising contacting a polymerizing agent, a primer-template duplex and a modified nucleotide of this invention in an extension solution, detecting changes of a detectable property of a detectable group on the modified nucleotide and/or the polymerizing agent and/or the primer-template duplex before, during and/or after one or a plurality of binding and/or incorporation events and analyzing the detected events to generate a sequence of incorporated nucleotides complementary to the template. The system and apparatus includes a reaction volume, a detector for detecting events occurring within a view field or volume, and an analyzer including software sufficient for converting the detected events into a sequence of incorporated nucleotides complementary to the template.

The present invention also provides a method, system, apparatus and composition for monitoring reactions between a modified phosphorylated biomolecule of this invention and its substrate.

### Compositions

The present invention also provides compositions including a molecular core, a first plurality of attachment sites extending out from the core, a second plurality of biomolecules, each biomolecule including a phosphate-containing group, where each biomolecule is attached to an attachment site of the molecular core through a direct bond to a terminal phosphate moiety of the phosphate-containing group or through a linker interposed between the site and the terminal phosphate moiety of the phosphate-containing group. In certain embodiments, the core includes one quencher or a plurality of quenchers. In other embodiments, the core includes an acceptor fluorophore or a plurality of acceptor fluorophore. In other embodiments, the core includes an acceptor fluorophore or a plurality of acceptor fluorophore and a donor fluorophore or a plurality of donor fluorophores. The core can be selected from the group consisting of boron-nitride nanostructures, carbon nanostructures, dendrimers, oligomers having multiple functional groups, polymers having multiple functional groups, metal oxide nanostructures (e.g., FeO, SiO₂, Al₂O₃, TiO2, ZnO, aluminosilicates, silicoaluminates, etc.), quantum dots (e.g., CdSe, etc.), metal clusters (e.g., non-transition metals, transition metals, actinide metals, lanthanide metals, etc. or mixed metal clusters), nanoshells (e.g., metal coated dielectric nanoparticles, metal coated metal nanoparticles, etc.), liposomes, or any other structure that can support attachment of a plurality of nucleotides through their terminal phosphate or mixtures of combinations thereof. The acceptor fluorophore can be any acceptor fluorophore set forth herein or any other acceptor fluorophore capable to undergo FRET with an appropriate donor fluorophore. The donor fluorophore can be any donor fluorophore set forth herein or any other donor fluorophore capable to undergo FRET with an appropriate acceptor fluorophore. The quencher can be any quencher set forth herein or any other agent that can quench the fluorescence of a fluorophore. The linker can be any linking molecular set forth here in or any other agent that can attach one molecular entity to another. In certain embodiments, the biomolecules can be the same or different. In other embodiments, the linkers can be the same or different. In certain embodiments, the quenchers can be the same or different. In certain embodiments, the acceptor fluorophores can be the same or different. In certain embodiments, the acceptor fluorophores can be the same or different and the donor fluorophores can be the same of different.

For nucleic acid sequencing or other reactions involving nucleotides, the structures are terminated by nucleotides. The compositions for carrying out the reaction include immobilized replication complexes (polymerase/primer/template) and a concentration of star molecules, each having a different attached dNTP type. If the polymerase includes a donor label, then the star molecules will include an acceptor and the FRET interaction will be between the donor on or associated with the polymerase or other type of polymerizing agent and the acceptor of the star molecule. If the star molecules are sufficiently large so that only a single star molecule can be at a replication site at a time, then the donor need not have a donor and the reaction can be followed simply by following the star molecule with incorporation events (binding and true incorporation events) when the star stays into the vicinity of the complex. The star molecules can either include a single fluorophore or a FRET active, donor-acceptor pair. Of course, in many embodiments, the replication complex includes a donor or is associated with a moiety or a separate structure that has a detectable property such as a fluorescently active quantum dot. By using star molecules having different fluorophores or different FRET active, donor-acceptor pairs, the identity of a base can be determined. Otherwise, the identity can be determined by the molecular dynamics, i.e., each dNTP can be modified so that each has an incorporation event duration that is unique so that a single fluorophore or FRET active, donor-acceptor pair can be used and identity coming from the duration of time the molecule spends within the vicinity of the replication complex.

For star molecules having a donor in the core and acceptor modified dNTPs attached at the terminal positions, the donor can be a donor that is excited by a persistent donor associated with or bonded to a replicating complex such as a quantum dot so that the core donor is not excited until it is in the immediate vicinity of the persistent donor. Once proximity is established (moves into the immediate vicinity of the replicating complex associated with or attached to the persistent donor), the donor is excited and in turn transfers energy to the acceptor. By monitoring the duration that the star molecule states in the vicinity of the replicating complex, base incorporation can be verified and the dNTP identity can be established either by the duration and/or wavelength of the triplet FRET emission. Triple FRET means that energy transfer to the acceptor does not occur via the persistent donor, but by the core donor. Thus, energy is first accepted by the persistent donor and transferred to the intermediate (core) donor and onto an acceptor to undergo FRET with the core donor. Once energy is transferred from the persistent donor to the core donor (an acceptor relative to the persistent donor), the core donor either transfers energy to the acceptor or fluoresces or does both.

These same basic principals can be applied to any reacting system where one desires to reduce background by limiting the number of fluorophores, while increasing the number of substrate molecules within the reaction volume.

### BRIEF DESCRIPTION OF THE DRAWINGS

The invention can be better understood with reference to the following detailed description together with the appended illustrative drawings in which like elements are numbered the same:

FIG. 1 depicts the preparation of dATP γ-ester, dATP-6-Cbz prepared using a method of this invention, where a phosphate serves as the nucleophilic agent to displace a suitable leaving group.

FIG. 2 depicts a synthetic scheme for preparing dual labeled dNTPs and a two dNTP functionalized label, a molecule that increases local dNTP concentration at constant dye concentration.

FIGS. 3A&B depict pictures of TLC plates showing synthesis products.

FIG. 4 depicts an embodiment of a star molecule, a molecule having two or more dNTPs attached thereto, synthetic scheme.

FIG. 5 depicts two examples of core structures used to prepare star molecules.

FIGS. 6A&B depict mass spectra of dA-L2-Cy3-L2-Cy5, a dual labeled dNTP and of dA-L2-Cy3-L2-dA, a two dNTP functionalized dye.

FIG. 7 depicts results of primer extension reactions of dA-L2-Cy3, a dATP linked with Cy3 via the gamma phosphate and of dA-L2-Cy3-L2-dA, “*dA2Cy3”, a two dNTP functionalized dye.

FIG. 8 depicts results of pictures of TLC plates of the reactions of FIG. 8.

FIG. 9 depicts results of primer extension reactions of “*dA2Cy3” (dA-L2-Cy3-L2-dA, a two dNTP functionalized dye).

FIG. 10 depicts results of primer extension reactions of dA-L2-Cy3, a gamma labeled dNTP and of ‘*dA2Cy3’ (dA-L2-Cy3-L2-dA, a two dNTP functionalized dye).

FIG. 11 depicts results of primer extension reactions of dA-L2-Cy3, a dATP linked with Cy3 via the gamma phosphate, either alone or with gamma-labeled dGTP-L2-Al610, and of ‘*dA2Cy3’ (dA-L2-Cy3-L2-dAa two dNTP functionalized dye) either alone or with gamma-labeled dGTP-L2-Al610. Reactions were performed on the indicated template with the indicated nucleotide amounts.

FIG. 12A depicts results of primer extension reactions of dA-L2-Cy3, a gamma labeled dNTP and of ‘*dA2Cy3’ (dA-L2-Cy3-L2-dA, a two dNTP functionalized dye) with several polymerase variants.

FIG. 12B depicts results of primer extension reactions of dA-L2-Cy3-L2-Cy5.

FIG. 12C depicts a graph showing the spectra of a quantum dot QDot 525, Cy3, Cy5 and 5-ROX illustrating a triple FRET sequencing strategy.

FIG. 13 depicts an embodiment of a dendrimer structure having as central group Z having a detectable property and arms terminating in an L-dNTP moeity.

FIG. 14 depicts a set of the structures set forth in Table 1.

FIGS. 15A-X depict mass spectra of the compound of FIG. 14.

### DETAILED DESCRIPTION OF THE INVENTION

The inventors have invented a novel and general synthetic methodology for preparing modified biomolecules, where the biomolecule includes at least one phosphate group or a plurality of phosphate groups and the modification occurs at one or more of the phosphate groups. The inventors have found this methodology is ideally suited for preparing modified nucleotides and nucleotide analogs, especially modified nucleotides including non-persistent or non-persistent and persistently labels, e.g., modified nucleotide tri, tetra, penta, hexa, hepta, etc. phosphates modified at one or more of the phosphate groups, which can be additionally modified on a persistent portion of the nucleotide such as the base. The inventors have found that a biomolecule including at least one phosphate group such as a nucleotide can be contacted with a modifying agent including a leaving group adapted to be displaced under mild conditions by the phosphate group to form modified biomolecules including the modifying agent save for the leaving group. The modifying agent can include a group having a detectable property sometimes called the detectable group or a quencher. If the modifying agent is a linker, then the methodology can further include the step of reacting the linker modified biomolecule with a detectable group to form a biomolecule with a detectable group bonded to the biomolecule via a linker. For quencher modified nucleotides, the quencher is adapted to quench donor fluorescence of a donor attached to a polymerase or any other compound capable of templated specific nucleotide addition. The donor quenching can then be correlated to binding and incorporation events to yield a sequence of base additions.

The present invention broadly relates to a class of molecules bearing one and generally a plurality of biomolecules including one or a plurality of phosphate moieties (—OP(O)(OH)O—) or analogs thereof attached to a molecular core of the molecule through its terminal phosphate moiety. Molecules bearing multiple biomolecules are sometimes referred to herein as star molecules.

The present invention broadly relates to a method for preparing a modified biomolecules including at least one phosphate group including the step of contacting the biomolecule and a modifying agent having a leaving group capable of being displaced by the at least one phosphate under displacement reaction conditions to form a phosphate modified biomolecule.

Sequencing with Quencher Modified Nucleotides

The modified nucleotides where the modifying agent includes a group or moiety that is capable or designed to quench a fluorescent property of a fluorophore associated with, located near, or bonded to a polymerizing agent such as a polymerase including normal polymerases or transcriptases or any other agent that can stepwise extend a primer relative to a template, to the primer, and/or to the template. For two quencher systems, two modified nucleotide types would include different quenchers having different quenching efficiencies for the fluorophore. For three quencher systems, three modified nucleotide types would include different quenchers having different quenching efficiencies for the fluorophore. For four quencher systems, four modifiers nucleotide types would include different quenchers having different quenching efficiencies for the fluorophore.

Alternatively, the a polymerizing agent, the primer and/or the template can include different fluorophores associated with, located near, or bonded to so that the quencher on the modified nucleotide types of this invention will differently quench the different fluorophores.

### Compositions Formulas

The present invention relates to phosphate modified biomolecules of the following structure:

Z′-(Z-BioM)ₙ (I)

where Z′ is a carbyl group or a group having a detectable property or a group capable of interfering with, quenching, augmenting, reducing or enhancing a detectable property of a group on another biomolecule, biomolecular complex or biomolecular assembly, Z is a phosphate-containing group including one or a plurality of phosphate moieties or a synthetic analogs thereof, BioM is a biomolecule, and n is an integer have a value between 1 and 10.

The present invention relates to phosphate modified biomolecules of the following structure:

Z′-Z″-(Z-BioM)ₘ (II)

where Z′ is a carbyl group or a group having a detectable property or a group capable of interfering with, quenching, augmenting, reducing or enhancing a detectable property of a group on another biomolecule, biomolecular complex or biomolecular assembly, Z″ is a multi-functional group, Z is a phosphate-containing group including one or a plurality of phosphate groups or a synthetic analogs thereof, BioM is a biomolecule, and m is an integer have a value between 1 and 1000.

The present invention relates to phosphate modified biomolecules of the following structure:

Z′-[Z″-(Z-BioM)ₘ]ᵢ (II)

where Z′ is a carbyl group or a group having a detectable property or a group capable of interfering with, quenching, augmenting, reducing or enhancing a detectable property of a group on another biomolecule, biomolecular complex or biomolecular assembly, Z″ is a multi-functional group, Z is a phosphate-containing group including one or a plurality of phosphate moieties or a synthetic analogs thereof, BioM is a biomolecule, m is an integer have a value between 1 and 1000, and i is an integer having a have between 1 and 1000.

The present invention relates to phosphate modified biomolecules of the following structure:

Z′-(L-Z-BioM)ₙ (IV)

where Z′ is a carbyl group or a group having a detectable property or a group capable of interfering with, quenching, augmenting, reducing or enhancing a detectable property of a group on another biomolecule, biomolecular complex or biomolecular assembly, L is a linker or linking group, Z is a phosphate-containing group including one or a plurality of phosphate moieties or a synthetic analogs thereof, BioM is a biomolecule, and n is an integer have a value between 1 and 10.

The present invention relates to phosphate modified biomolecules of the following structure:

Z′-Z″-(L-Z-BioM)ₘ (V)

where Z′ is a carbyl group or a group having a detectable property or a group capable of interfering with, quenching, augmenting, reducing or enhancing a detectable property of a group on another biomolecule, biomolecular complex or biomolecular assembly, Z″ is a multi-functional group, L is a linker or linking group, Z is a phosphate-containing group including one or a plurality of phosphate moieties or a synthetic analogs thereof, BioM is a biomolecule, and m is an integer have a value between 1 and 1000.

The present invention relates to phosphate modified biomolecules of the following structure:

Z′-[Z″-(L-Z-BioM)ₘ]ᵢ (VI)

where Z′ is a carbyl group or a group having a detectable property or a group capable of interfering with, quenching, augmenting, reducing or enhancing a detectable property of a group on another biomolecule, biomolecular complex or biomolecular assembly, Z″ is a multi-functional group, L is a linker or linking group, Z is a phosphate-containing group including one or a plurality of phosphate moieties or a synthetic analogs thereof, BioM is a biomolecule, m is an integer have a value between 1 and 1000, and i is an integer having a have between 1 and 1000.

The present invention relates to phosphate modified biomolecules of the following structure:

Z′-L′-Z″-(L-Z-BioM)ₘ (VII)

where Z′ is a carbyl group or a group having a detectable property or a group capable of interfering with, quenching, augmenting, reducing or enhancing a detectable property of a group on another biomolecule, biomolecular complex or biomolecular assembly, L′ is a second linker or linking group, Z″ is a multi-functional group, L is a first linker or linking group, Z is a phosphate-containing group including one or a plurality of phosphate moieties or a synthetic analogs thereof, BioM is a biomolecule, and m is an integer have a value between 1 and 1000.

The present invention relates to phosphate modified biomolecules of the following structure:

Z′-L′-[Z″-(L-Z-BioM)ₘ]ᵢ (VIII)

where Z′ is a carbyl group or a group having a detectable property or a group capable of interfering with, quenching, augmenting, reducing or enhancing a detectable property of a group on another biomolecule, biomolecular complex or biomolecular assembly, L′ is a second linker or linking group, Z″ is a multi-functional group, L is a first linker or linking group, Z is a phosphate-containing group including one or a plurality of phosphate moieties or a synthetic analogs thereof, BioM is a biomolecule, m is an integer have a value between 1 and 1000, and i is an integer having a have between 1 and 1000.

The present invention relates to phosphate modified biomolecules of the following structure:

Z′-[L′-Z″-(L-Z-BioM)ₘ]ᵢ (IX)

where Z′ is a carbyl group or a group having a detectable property or a group capable of interfering with, quenching, augmenting, reducing or enhancing a detectable property of a group on another biomolecule, biomolecular complex or biomolecular assembly, L′ is a second linker or linking group, Z″ is a multi-functional group, L is a first linker or linking group, Z is a phosphate-containing group including one or a plurality of phosphate moieties or a synthetic analogs thereof, BioM is a biomolecule, m is an integer have a value between 1 and 1000, and i is an integer having a have between 1 and 1000.

The present invention relates to phosphate modified biomolecules of the following structure:

Z′-Z′″-(Z-BioM)ₙ (X)

where Z′ is a first group, Z′″ is a second group, Z is a phosphate-containing group including one or a plurality of phosphate moieties or a synthetic analogs thereof, BioM is a biomolecule, n is an integer have a value between 1 and 10, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-Z′″-Z″-(Z-BioM)ₘ (XI)

where Z′ is a first group, Z′″ is a second group, Z″ is a multi-functional group, Z is a phosphate-containing group including one or a plurality of phosphate moieties or a synthetic analogs thereof, BioM is a biomolecule, m is an integer have a value between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-Z′″-[Z″-(Z-BioM)ₘ]ᵢ (XII)

where Z′ is a first group, Z′″ is a second group, Z″ is a multi-functional group, Z is a phosphate-containing group including one or a plurality of phosphate moieties or a synthetic analogs thereof, BioM is a biomolecule, m is an integer have a value between 1 and 1000, i is an integer having a have between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-Z′″-(L-Z-BioM)ₙ (XIII)

where Z′ is a first group, Z′″ is a second group, L is a linker or linking group, Z is a phosphate-containing group including one or a plurality of phosphate moieties or a synthetic analogs thereof, BioM is a biomolecule, n is an integer have a value between 1 and 10, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-Z′″-[Z″-(L-Z-BioM)ₘ]ᵢ (XIV)

where Z′ is a first group, Z′″ is a second group, Z″ is a multi-functional group, L is a linker or linking group, Z is a phosphate-containing group including one or a plurality of phosphate moieties or a synthetic analogs thereof, BioMis a biomolecule, m is an integer have a value between 1 and 1000, i is an integer having a have between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-L′-Z′″-(L-Z-BioM)ₙ (XV)

where Z′ is a first group, L′ is a second linker or linking group, Z′″ is a second group, L is a first linker or linking group, Z is a phosphate-containing group including one or a plurality of phosphate moieties or a synthetic analogs thereof, BioM is a biomolecule, n is an integer have a value between 1 and 10, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-L′-Z′″-Z″-(L-Z-BioM)ₘ (XVI)

where Z′ is a first group, L′ is a second linker or linking group, Z′″ is a second group, Z″ is a multi-functional group, L is a first linker or linking group, Z is a phosphate-containing group including one or a plurality of phosphate moieties or a synthetic analogs thereof, BioM is a biomolecule, m is an integer have a value between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-L′-Z′″-[Z″-(L-Z-BioM)ₘ]ᵢ (XVII)

where Z′ is a first group, L′ is a second linker or linking group, Z′″ is a second group, Z″ is a multi-functional group, L is a first linker or linking group, Z is a phosphate-containing group including one or a plurality of phosphate groups or a synthetic analogs thereof, BioM is a biomolecule, m is an integer have a value between 1 and 1000, i is an integer having a have between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-L′-Z′″-L″-Z″-(L-Z-BioM)ₘ (XVIII)

where Z′ is first group, L′ is a second linker or linking group, Z′″ is a second group, L″ is a third linker or linking group, Z″ is a multi-functional group, L is a first linker or linking group, Z is a phosphate-containing group including one or a plurality of phosphate moieties or a synthetic analogs thereof, BioM is a biomolecule, m is an integer have a value between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-L′-Z′″-L″-[Z″-(L-Z-BioM)ₘ]ᵢ (XIX)

where Z′ is a first group, L′ is a second linker or linking group, Z′″ is a second group, L″ is a third linker or linking group, Z″ is a multi-functional group, L is a first linker or linking group, Z is a phosphate-containing group including one or a plurality of phosphate moieties or a synthetic analogs thereof, BioM is a biomolecule, m is an integer have a value between 1 and 1000, i is an integer having a have between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-L′-Z′″-[L″-Z″-(L-Z-BioM)ₘ]ᵢ (XX)

where Z′ is a first group, L′ is a second linker or linking group, Z′″ is a second group, L″ is a third linker or linking group, Z″ is a multi-functional group, L is a first linker or linking group, Z is a phosphate-containing group including one or a plurality of phosphate moieties or a synthetic analogs thereof, BioM is a biomolecule, m is an integer have a value between 1 and 1000, i is an integer having a have between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-Z″-(Z′″-Z-BioM)ₘ (XXI)

where Z′ is a first group, Z″ is a multi-functional group, Z′″ is a second group, Z is a phosphate-containing group including one or a plurality of phosphate moieties or a synthetic analogs thereof, BioM is a biomolecule, m is an integer have a value between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-Z″-(Z′″-L-Z-BioM)ₘ (XXII)

where Z′ is a first group, Z′″ is a multi-functional group, Z′″ is a second group, L is a first linker or linking group, Z is a phosphate-containing group including one or a plurality of phosphate moieties or a synthetic analogs thereof, BioM is a biomolecule, m is an integer have a value between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-Z″-(L′-Z′″-Z-BioM)ₘ (XXIII)

where Z′ is a first group, Z″ is a multi-functional group, Z′″ is a second group, L′ is a second linker or linking group, Z is a phosphate-containing group including one or a plurality of phosphate moieties or a synthetic analogs thereof, BioM is a biomolecule, m is an integer have a value between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-Z″-L′-(Z′″-L-Z-BioM)ₘ (XXIV)

where Z′ is a first group, Z″ is a multi-functional group, L′ is a second linker or linking group, Z′″ is a second group, optionally L is a first linker or linking group, Z is a phosphate-containing group including one or a plurality of phosphate moieties or a synthetic analogs thereof, BioM is a biomolecule, m is an integer have a value between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-L″-Z″-L′-(Z′″-L-Z-BioM)ₘ (XXV)

where Z′ is a first group, L″ is a third linker or linking group, Z″ is a multi-functional group, optionally L′ is a second linker or linking group, Z′″ is a second group, optionally L is a first linker or linking group, Z is a phosphate-containing group including one or a plurality of phosphate moieties or a synthetic analogs thereof, BioM is a biomolecule, m is an integer have a value between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-(Z-Nu)ₙ (XXVI)

where Z′ is a carbyl group R² group or a group having a detectable property or a group capable of interfering with, quenching, augmenting, reducing or enhancing a detectable property of a group on another biomolecule, biomolecular complex or biomolecular assembly, Z is a phosphate-containing group including one or a plurality of phosphate moieties or a synthetic analogs thereof, Nu is a nucleoside or nucleoside analog, and n is an integer have a value between 1 and 10.

The present invention relates to phosphate modified biomolecules of the following structure:

Z′-Z″-(Z-Nu)ₘ (XXVII)

where Z′ is a carbyl group or a group having a detectable property or a group capable of interfering with, quenching, augmenting, reducing or enhancing a detectable property of a group on another biomolecule, biomolecular complex or biomolecular assembly, Z″ is a multi-functional group, Z is a phosphate-containing group including one or a plurality of phosphate moieties or a synthetic analogs thereof, Nu is a nucleoside or nucleoside analog, and m is an integer have a value between 1 and 1000.

The present invention relates to phosphate modified biomolecules of the following structure:

Z′-[Z″Z-(Z-Nu)ₘ]ᵢ (XXVIII)

where Z′ is a carbyl group or a group having a detectable property or a group capable of interfering with, quenching, augmenting, reducing or enhancing a detectable property of a group on another biomolecule, biomolecular complex or biomolecular assembly, Z″ is a multi-functional group, Z is a phosphate-containing group including one or a plurality of phosphate moieties or a synthetic analogs thereof, Nu is a nucleoside or nucleoside analog, m is an integer have a value between 1 and 1000, and i is an integer having a have between 1 and 1000.

The present invention relates to phosphate modified biomolecules of the following structure:

Z′-(L-Z-NU)ₙ (XXIX)

where Z′ is a carbyl group or a group having a detectable property or a group capable of interfering with, quenching, augmenting, reducing or enhancing a detectable property of a group on another biomolecule, biomolecular complex or biomolecular assembly, L is a linker or linking group, Z is a phosphate-containing group including one or a plurality of phosphate moieties or a synthetic analogs thereof, Nu is a nucleoside or nucleoside analog, and n is an integer have a value between 1 and 10.

The present invention relates to phosphate modified biomolecules of the following structure:

Z′-Z″-(L-Z-Nu)ₘ (XXX)

where Z′ is a carbyl group or a group having a detectable property or a group capable of interfering with, quenching, augmenting, reducing or enhancing a detectable property of a group on another biomolecule, biomolecular complex or biomolecular assembly, Z″ is a multi-functional group, L is a linker or linking group, Z is a phosphate-containing group including one or a plurality of phosphate moieties or a synthetic analogs thereof, Nu is a nucleoside or nucleoside analog, and m is an integer have a value between 1 and 1000.

The present invention relates to phosphate modified biomolecules of the following structure:

Z′-[Z″-(L-Z-NU)ₘ]ᵢ (XXXI)

where Z′ is a carbyl group or a group having a detectable property or a group capable of interfering with, quenching, augmenting, reducing or enhancing a detectable property of a group on another biomolecule, biomolecular complex or biomolecular assembly, Z″ is a multi-functional group, L is a linker or linking group, Z is a phosphate-containing group including one or a plurality of phosphate moieties or a synthetic analogs thereof, Nu is a nucleoside or nucleoside analog, m is an integer have a value between 1 and 1000, and i is an integer having a have between 1 and 1000.

The present invention relates to phosphate modified biomolecules of the following structure:

Z′-L′-Z″-(L-Z-Nu)ₘ (XXXII)

where Z′ is a carbyl group or a group having a detectable property or a group capable of interfering with, quenching, augmenting, reducing or enhancing a detectable property of a group on another biomolecule, biomolecular complex or biomolecular assembly, L′ is a second linker or linking group, Z″ is a multi-functional group, L is a first linker or linking group, Z is a phosphate-containing group including one or a plurality of phosphate moieties or a synthetic analogs thereof, Nu is a nucleoside or nucleoside analog, and m is an integer have a value between 1 and 1000.

The present invention relates to phosphate modified biomolecules of the following structure:

Z′-L′-[Z″-(L-Z-NU)ₘ]ᵢ (XXXIII)

where Z′ is a carbyl group or a group having a detectable property or a group capable of interfering with, quenching, augmenting, reducing or enhancing a detectable property of a group on another biomolecule, biomolecular complex or biomolecular assembly, L′ is a second linker or linking group, Z″ is a multi-functional group, L is a first linker or linking group, Z is a phosphate-containing group including one or a plurality of phosphate moieties or a synthetic analogs thereof, Nu is a nucleoside or nucleoside analog, m is an integer have a value between 1 and 1000, and i is an integer having a have between 1 and 1000.

The present invention relates to phosphate modified biomolecules of the following structure:

Z′-[L′-Z″-(L-Z-Nu)ₘ]ᵢ (XXXIV)

where Z′ is a carbyl group or a group having a detectable property or a group capable of interfering with, quenching, augmenting, reducing or enhancing a detectable property of a group on another biomolecule, biomolecular complex or biomolecular assembly, L′ is a second linker or linking group, Z″ is a multi-functional group, L is a first linker or linking group, Z is a phosphate-containing group including one or a plurality of phosphate moieties or a synthetic analogs thereof, Nu is a nucleoside or nucleoside analog, m is an integer have a value between 1 and 1000, and i is an integer having a have between 1 and 1000.

The present invention relates to phosphate modified biomolecules of the following structure:

Z′-Z′″-(Z-Nu)ₙ (XXXV)

where Z′ is a first group, Z′″ is a second group, Z is a phosphate-containing group including one or a plurality of phosphate moieties or a synthetic analogs thereof, Nu is a nucleoside or nucleoside analog, n is an integer have a value between 1 and 10, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-Z′″-Z″-(Z-Nu)ₘ (XXXVI)

where Z′ is a first group, Z′″ is a second group, Z″ is a multi-functional group, Z is a phosphate-containing group including one or a plurality of phosphate moieties or a synthetic analogs thereof, Nu is a nucleoside or nucleoside analog, m is an integer have a value between 1 and 1000, i is an integer having a have between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-Z′″-[Z″-(Z-Nu)ₘ]ᵢ (XXXVII)

where Z′ is a first group, Z′″ is a second group, Z″ is a multi-functional group, Z is a phosphate-containing group including one or a plurality of phosphate moieties or a synthetic analogs thereof, Nu is a nucleoside or nucleoside analog, m is an integer have a value between 1 and 1000, i is an integer having a have between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-Z′″-(L-Z-Nu)ₙ (XXXVIII)

where Z′ is a first group, Z′″ is a second group, L is a linker or linking group, Z is a phosphate-containing group including one or a plurality of phosphate moieties or a synthetic analogs thereof, Nu is a nucleoside or nucleoside analog, n is an integer have a value between 1 and 10, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-Z′″-[Z″-(L-Z-Nu)ₘ]ᵢ (XXXIX)

where Z′ is a first group, Z′″ is a second group, Z″ is a multi-functional group, L is a linker or linking group, Z is a phosphate-containing group including one or a plurality of phosphate moieties or a synthetic analogs thereof, Nu is a nucleoside or nucleoside analog, m is an integer have a value between 1 and 1000, i is an integer having a have between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure

Z′-L′-Z′″-(L-Z-Nu)ₙ (XL)

where Z′ is a first group, L′ is a second linker or linking group, Z′″ is a second group, L is a first linker or linking group, Z is a phosphate-containing group including one or a plurality of phosphate moieties or a synthetic analogs thereof, Nu is a nucleoside or nucleoside analog, n is an integer have a value between 1 and 10, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-L′-Z′″-Z″-(L-Z-Nu)ₘ (XLI)

where Z′ is a first group, L′ is a second linker or linking group, Z′″ is a second group, Z″ is a multi-functional group, L is a first linker or linking group, Z is a phosphate-containing group including one or a plurality of phosphate moieties or a synthetic analogs thereof, Nu is a nucleoside or nucleoside analog, m is an integer have a value between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-L′-Z′″-[Z″-(L-Z-Nu)ₘ]ᵢ (XLII)

where Z′ is a first group, L′ is a second linker or linking group, Z′″ is a second group, Z″ is a multi-functional group, L is a first linker or linking group, Z is a phosphate-containing group including one or a plurality of phosphate moieties or a synthetic analogs thereof, Nu is a nucleoside or nucleoside analog, m is an integer have a value between 1 and 1000, i is an integer having a have between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-L′-Z′″-L″-Z″-(L-Z-Nu)ₘ (XLIII)

where Z′ is a first group, L′ is a second linker or linking group, Z′″ is a second group, L″ is a third linker or linking group, Z″ is a multi-functional group, L is a first linker or linking group, Z is a phosphate-containing group including one or a plurality of phosphate moieties or a synthetic analogs thereof, Nu is a nucleoside or nucleoside analog, m is an integer have a value between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-L′-Z′″-L″-[Z″-(L-Z-Nu)ₘ]ᵢ (XLIV)

where Z′ is a first group, L′ is a second linker or linking group, Z′″ is a second group, L″ is a third linker or linking group, Z″ is a multi-functional group, L is a first linker or linking group, Z is a phosphate-containing group including one or a plurality of phosphate moieties or a synthetic analogs thereof, Nu is a nucleoside or nucleoside analog, m is an integer have a value between 1 and 1000, is an integer having a have between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-L′-Z′″-[L″-Z″-(L-Z-Nu)ₘ]ᵢ (XLV)

where Z′ is a first group, L′ is a second linker or linking group, Z′″ is a second group, L″ is a third linker or linking group, Z″ is a multi-functional group, L is a first linker or linking group, Z is a phosphate-containing group including one or a plurality of phosphate moieties or a synthetic analogs thereof, Nu is a nucleoside or nucleoside analog, m is an integer have a value between 1 and 1000, is an integer having a have between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-Z″-(Z′″-Z-Nu)ₘ (XLVI)

where Z′ is a first group, Z″ is a multi-functional group, Z′″ is a second group, Z is a phosphate-containing group including one or a plurality of phosphate moieties or a synthetic analogs thereof, Nu is a nucleoside or nucleoside analog, m is an integer have a value between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-Z″-(Z′″-L-Z-Nu)ₘ (XLVII)

where Z′ is a first group, Z″ is a multi-functional group, Z′″ is a second group, L is a first linker or linking group, Z is a phosphate-containing group including one or a plurality of phosphate moieties or a synthetic analogs thereof, Nu is a nucleoside or nucleoside analog, m is an integer have a value between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-Z″(L′-Z′″-Z-Nu)ₘ (XLVIII)

where Z′ is a first group, Z″ is a multi-functional group, Z′″ is a second group, L′ is a second linker or linking group, Z is a phosphate-containing group including one or a plurality of phosphate moieties or a synthetic analogs thereof, Nu is a nucleoside or nucleoside analog, m is an integer have a value between 1 and 1000, and the first group Z′ and the second group Z′″ form a coupled pair;

Z′-Z″-L′-(Z′″-L-Z-Nu)ₘ (XLIX)

where Z′ is a first group, Z″ is a multi-functional group, L′ is a second linker or linking group, Z′″ is a second group, optionally L is a first linker or linking group, Z is a phosphate-containing group including one or a plurality of phosphate moieties or a synthetic analogs thereof, Nu is a nucleoside or nucleoside analog, m is an integer have a value between 1 and 1000, and the first group Z′ and the second group Z′″ form a coupled pair at least one of which includes a group having a detectable property or a group capable of interfering with, quenching, augmenting, reducing or enhancing a detectable property of a group on another biomolecule, biomolecular complex or biomolecular assembly, where a coupled pair comprises two molecular structures that are bond to each other via covalent, ionic, dipolar, apolar and/or any other physical or chemical interaction.

The present invention relates to phosphate modified biomolecules of the following structure:

Z′-L″-Z″-L′-(Z′″-L-Z-Nu)ₘ (L)

where Z′ is a first group, L″ is a third linker or linking group, Z″ is a multi-functional group, optionally L′ is a second linker or linking group, Z′″ is a second group, optionally L is a first linker or linking group, Z is a phosphate-containing group including one or a plurality of phosphate moieties or a synthetic analogs thereof, Nu is a nucleoside or nucleoside analog, m is an integer have a value between 1 and 1000, and the first group Z′ and the second group Z′″ form a coupled pair.

In all of the formulas set forth above, the linkers L, L′, and L″ can be the same or different; and the Z″ and Z′″ can be the same or different. Although the inventors have attempted to set forth schematically a large number of phosphate modified biomolecules, phosphorylated biomolecules modified through a phosphate group, any structure including a plurality of biomolecules linked to the structure through a phosphate or phosphate analog group at terminal sites of the structure are also contemplated by this invention. In embodiments involving FRET interaction between a fluorophore in the center or core portion of a multi-armed structure, the biomolecules should be within the FRET distance of the core. Generally, the FRET distance is a volume centered about the immobilized fluorophore, where the volume has a radius of at most about 100 Å, but can be a larger depending on the environment, the FRET pair, etc. In certain embodiments, the FRET pair are with a distance of about 10 to about 100 Å of the core. In certain embodiments, the FRET pair are with a distance of about 10 to about 90 Å of the core. In certain embodiments, the FRET pair are with a distance of about 10 to about 80 Å of the core. In certain embodiments, the FRET pair are with a distance of about 10 to about 70 Å of the core. In certain embodiments, the FRET pair are with a distance of about 10 to about 60 Å of the core. In certain embodiments, the FRET pair are with a distance of about 10 to about 50 Å of the core. In certain embodiments, the FRET pair are with a distance of about 10 to about 40 Å of the core. In certain embodiments, the FRET pair are with a distance of about 10 to about 30 Å of the core. In certain embodiments, the FRET pair are with a distance of about 10 to about 20 Å of the core. Of course, each biomolecule can be the same or different distance from the core, provided that the distance is within the specified distance of one of the FRET geared structures. Similar distance considerations also pertain to star molecules have a FRET active, donor-acceptor in the core of the star molecular structure. Generally, the acceptor is situated on the source of nucleotides the modified nucleotides in the case; while the donor is situated near or on, associated with or bonded to the polymerizing agent and/or the primer-template duplex.

The star molecules of this invention are well suited for use in a direct detection of sequence information. For example, the star molecules can be used without using labeled polymerase due to a significant reduction background due to a high relative concentration of a given dNTP and a low relative concentration of fluorophore, only one per star molecule. Thus, a star molecule including a single fluorophore and arms, where each arm includes a nucleotides capable of incorporation by a replication complex, can be traced as it proceeds through a viewing field of a detector. If the star molecule encounters an immobilized or confined replication complex that requires the nucleotide at the end of the arms of the star molecule, then the star molecule would be fixed at the location of the immobilized or confined replication complex for a time sufficient to either unproductively bind to or be incorporated by the complex, providing direct information on sequencing activity of the complex. By making the size of the star molecules large enough, the inventors believe that only one star molecule will be present at a given replication complex, thus permitting base by base incorporation data to be directly detected.

Besides a star molecule with a single fluorophore, the star molecule can include a single donor-acceptor pair and arms, where each arm includes a nucleotide capable of incorporation by a replication complex, can be traced as it proceeds through a viewing field of a detector. By using such a star molecule, a single excitation laser can still be used in a 4 color dNPT scheme, one color for each dNTP type. Such a star molecule could be viewed as a donor-acceptor-amplifier-dNTP. Thus, the structure could be viewed as a flower, where the stem includes the donor, the base of the flower including the acceptor and the petals including attached dNTP—as many as needed to obtain the desired effect. Consistent association of signal with a particular location and emission features detected during duration at the location are used to determine sequence information.

Star molecules allow us to reduce the background noise and still work with an effectively high concentration of nucleotide. This is in stark contrast to what most other scientists would prefer to do. Other researches do whatever they can to increase the signal from their molecule often preferring molecules onto which many fluorophores can be attached.

The inclusion of star molecules in sequencing reactions enables high nucleotide concentrations and low fluorophore concentrations to be added to a reaction. The use of molecules including a single fluorophore or donor-acceptor pair and two or more nucleotides effectively increases the nucleotide concentration by the number of nucleotide per molecule. For example, if one desired to sequence using a reaction mixtures including 1 micromolar nucleotide concentration, one can add just 0.5 micromolar of molecule including two nucleotides and one fluorophore such as a dA-PPP-linker-Fluorophore-linker-PPP-dA (P represents phosphate or a phosphate analog), effectively reducing the amount of fluorophore added into the reaction by half, while maintaining the same nucleotide concentration. Alternatively, if one prefers to increase nucleotide concentration to 2 micromolar, one can add 1 micromolar of a dA-PPP-linker-Fluorophore-linker-PPP-dA molecule (P represents phosphate or a phosphate analog), effectively doubling the amount of nucleotide added into the reaction while maintaining the concentration of acceptor fluorophore. Similarly, for higher-order star molecules, the effective nucleotide concentration can be n times the fluorophore concentration, where n is the number of nucleotides attached to arms of the star molecule.

The number of arms in a star molecule including a single fluorophore or donor-acceptor pair is a matter of design, solubility, mobility, steric bulk, electrostatic, other physical and/or chemical factors. For example, we have a detectable molecule that is compatible with our sequencing technology, i.e., an acceptor fluorophore such as Alexa610 that is an acceptable FRET partner with a donor used in our sequencing system (i.e., Alexa488). Acceptor fluorophores are chosen such that they remain active for more extended periods of time, as they will undergo FRET with a donor multiple times. Preferably, the number of times that an acceptor fluorophore will undergo FRET reflects the number of monomers attached to the star molecule. Each nucleotide type is attached, preferably covalently, to a distinguishable acceptor fluorophore (i.e., there are at least 4 different types of star molecules in a reaction that is producing information about the sequence identity of 4 different base types in a DNA molecule). In certain circumstances, it may be desirable to use a subset of nucleotide types or fluorophores.). Acceptor fluorophores are distinguished by emission wavelength and/or duration and/or intensity. The increased size of the star molecule slow diffusion and increases the duration of base-specific signal, thereby facilitating accurate detection.

The molecule may consist of linked dNTPs without a central detectable moiety. This variant molecule type may be used in polymerase extension reactions to promote efficient DNA synthesis. If the star molecule is to be used in a PCR or other reaction that exposes it to heat or other extreme conditions, then the bonds attaching the dNTPs to the star should be heat stable (e.g., oxygen, carbon, etc.).

The molecule may include dNTPs attached to a center or molecular core including a detectable moiety via linkers. This variant molecule type is especially useful in single molecule sequencing reactions by promoting efficient DNA synthesis due to an apparent increase in nucleotide concentration. In certain embodiments, for stability purposes, the linker attaches to the dNTP via an oxygen or carbon atom. In other embodiments, the linker attaches to the dNTP via an oxygen or other atom.

In addition to the minimal ‘2 dimensional’ or dual star structure, nucleoside-PPP-linker-DetectabelMoiety-linker-PPP-nucleoside, where the P means phosphate, the term nucleoside encompasses deoxyribonucleoside and ribonucleosides (i.e., DATP, dGTP, dCTP, dTTP, dUTP, ATP, GTP, CTP, TTP, and analogs thereof), the molecule may be 3 dimensional including a large number of linker-PPP-nucleoside moieties. For example, the star structure may have a dendrimer type structure that is of variable size to allow for more or fewer monomer attachment sites, as needed, or a buckyball or other 3D structure with attachment sites on at least two sites with multiple attachments positions preferred. There may be more than 3 phosphates separating the nucleoside-linker and these phosphates may be considered to be part of the linker structure. To obtain similar efficiencies of incorporation, the preferred molecule for single molecule sequencing is symmetrical; however, asymmetrical molecules may also be used.

The minimal definition of a detectable star molecule is one that contains more than one nucleotide (monomer) linked via a linker to an atomic or detectable moiety which is in turn linked via a linker to another nucleotide (monomer). Atomic or detectable moieties include, without limitation, Europium shift agents, quantum dots, nanotube, nanoparticles, NMR active atoms or the like, any atomic element amenable to attachment to a specific site in a polymerizing agent or dNTP, especially fluorescent dyes such as d-Rhodamine acceptor dyes including dichloro[R110], dichloro[R6G], dichloro[TAMRA], dichloro [ROX] or the like, fluorescein donor dye including fluorescein, 6-FAM, or the like; Acridine including Acridine orange, Acridine yellow, Proflavin, pH 7, or the like; Aromatic Hydrocarbon including 2-Methylbenzoxazole, Ethyl p-dimethylaminobenzoate, Phenol, Pyrrole, benzene, toluene, or the like; Arylmethine Dyes including Auramine O, Crystal violet, H2O, Crystal violet, glycerol, Malachite Green or the like; Coumarin dyes including 7-Methoxycoumarin-4-acetic acid, Coumarin 1, Coumarin 30, Coumarin 314, Coumarin 343, Coumarin 6 or the like; Cyanine Dye including 1,1′-diethyl-2,2′-cyanine iodide, Cryptocyanine, Indocarbocyanine (Cy3)dye, Indodicarbocyanine (Cy5)dye, Indotricarbocyanine (Cy7)dye, Oxacarbocyanine (Cy3′)dye, Oxadicarbocyanine (Cy5′)dye, Oxatricarbocyanine (Cy7′)dye, Pinacyanol iodide, Stains all, Thiacarbocyanine (Cy3″)dye, ethanol, Thiacarbocyanine (Cy3″)dye, n-propanol, Thiadicarbocyanine (Cy5″)dye, Thiatricarbocyanine (Cy7″)dye, or the like; Dipyrrin dyes including N,N′-Difluoroboryl-1,9-dimethyl-5-(4-iodophenyl)-dipyrrin, N,N′-Difluoroboryl-1,9-dimethyl-5-[(4-(2-trimethylsilylethynyl), N,N′-Difluoroboryl-1,9-dimethyl-5-phenydipyrrin, or the like; Merocyanines including 4-(dicyanomethylene)-2-methyl-6-(p-dimethylaminost-yryl)₄H-pyran (DCM), acetonitrile, 4-(dicyanomethylene)-2-methyl-6-(p-dim-ethylaminostyryl)₄H-pyran (DCM), methanol, 4-Dimethylamino-4′-nitrostilbe-ne, Merocyanine 540, or the like; Miscellaneous Dye including 4′,6-Diamidino-2-phenylindole (DAPI), 4′,6-Diamidino-2-phenylindole (DAPI), dimethylsulfoxide, 7-Benzylamino-4-nitrobenz-2-oxa-1,3-diazole, Dansyl glycine, H2O, Dansyl glycine, dioxane, Hoechst 33258, DMF, Hoechst 33258, H2O, Lucifer yellow CH, Piroxicam, Quinine sulfate, 0.05 M H2SO4, Quinine sulfate, 0.5 M H2SO4, Squarylium dye III, or the like; Oligophenylenes including 2,5-Diphenyloxazole (PPO), Biphenyl, POPOP, p-Quaterphenyl, p-Terphenyl, or the like; Oxazines including Cresyl violet perchlorate, Nile Blue, methanol, Nile Red, Nile blue, ethanol, Oxazine 1, Oxazine 170, or the like; Polycyclic Aromatic Hydrocarbons including 9,10-Bis(phenylethynyl)anthracene, 9,10-Diphenylanthracene, Anthracene, Naphthalene, Perylene, Pyrene, or the like; polyene/polyynes including 1,2-diphenylacetylene, 1,4-diphenylbutadiene, 1,4-diphenylbutadiyne, 1,6-Diphenylhexatriene, Beta-carotene, Stilbene, or the like; Redox-active Chromophores including Anthraquinone, Azobenzene, Benzoquinone, Ferrocene, Riboflavin, Tris(2,2′-bipyridyl)ruth-enium(II), Tetrapyrrole, Bilirubin, Chlorophyll a, diethyl ether, Chlorophyll a, methanol, Chlorophyll b, Diprotonated-tetraphenylporphyrin-, Hematin, Magnesium octaethylporphyrin, Magnesium octaethylporphyrin (MgOEP), Magnesium phthalocyanine (MgPc), PrOH, Magnesium phthalocyanine (MgPc), pyridine, Magnesium tetramesitylporphyrin (MgTMP), Magnesium tetraphenylporphyrin (MgTPP), Octaethylporphyrin, Phthalocyanine (Pc), Porphin, Tetra-t-butylazaporphine, Tetra-t-butylnaphthalocyanine, Tetrakis(2,6-dichlorophenyl)porphyrin, Tetrakis(o-aminophenyl)porphyrin, Tetramesitylporphyrin (TMP), Tetraphenylporphyrin (TPP), Vitamin B12, Zinc octaethylporphyrin (ZnOEP), Zinc phthalocyanine (ZnPc), pyridine, Zinc tetramesitylporphyrin (ZnTMP), Zinc tetramesitylporphyrin radical cation, Zinc tetraphenylporphyrin (ZnTPP), or the like; Xanthenes including Eosin Y, Fluorescein, basic ethanol, Fluorescein, ethanol, Rhodamine 123, Rhodamine 6G, Rhodamine B, Rose bengal, Sulforhodamine 101, or the like; or mixtures or combination thereof or synthetic derivatives thereof or FRET fluorophore-quencher pairs including DLO-FB 1 (5′-FAM/3′-BHQ-1) DLO-TEB 1 (5′-TET/3′-BHQ-1), DLO-JB1 (5′-JOE/3′-BHQ-1), DLO-HB1 (5′-HEX/3′-BHQ-1), DLO-C3B2 (5′-Cy3/3′-BHQ-2), DLO-TAB2 (5′-TAMRA/3′-BHQ-2), DLO-RB2 (5′-ROX/3′-BHQ-2), DLO-C5B3(5′-Cy5/3′-BHQ-3), DLO-C55B3 (5′-Cy5.5/3′-BHQ-3), MBO-FB1 (5′-FAM/3′-BHQ-1), MBO-TEB1 (5′-TET/3′-BHQ-1), MBO-JB1 (5′-JOE/3′-BHQ-1), MBO-HB1 (5′-HEX/3′-BHQ-1), MBO-C3B2 (5′-Cy3/3′-BHQ-2), MBO-TAB2 (5′-TAMRA/3′-BHQ-2), MBO-RB2 (5′-ROX/3′-BHQ-2); MBO-C5B3 (5′-Cy5/3′-BHQ-3), MBO-C55B3 (5′-Cy5.5/3′-BHQ-3) or similar FRET pairs available from Biosearch Technologies, Inc. of Novato, Calif., tags with NMR active groups, tags with spectral features that can be easily identified such as near IR, IR, far IR, visible, UV, far UV, X-ray or the like, tags with quenching moieties, any other atomic or molecular entity that includes a detectable moiety o a quenching moiety or mixtures or combinations thereof.

The minimal definition of a star molecule lacking a detectable (i.e., fluorophore, chromophore, etc.) moiety is a nucleoside-PPPP-nucleoside. However, nucleoside-PPP-linker-PPP-nucleoside is also a preferred, non-detectable star molecule.

The star structure composition is applicable to deoxyribonucleotides, ribonucleotides, amino acids, tRNA, proteins, peptides, carbohydrates, any organic or inorganic monomer, and combinations thereof that are linked to form a unit that consists of at least two monomer types linked to a detectable (or not) moiety. A star molecule is stable to reaction conditions needed to promote monomer incorporation by a polymerizing agent. The number of monomer units attached to the star molecule is minimally two and can be maximally the number of units that can be added to the nucleating molecule, considering stearic and/or electrostatic and/or chemical constraints.

Different types of monomer units may be linked to the star molecule. This type of molecule is referred to as a hetero-star molecule.

For single molecule DNA sequencing the monomer units are preferably the same type (a homo-star molecule), but they may be linked to the star molecule by the same or different linking mechanisms or linker lengths. It may be advantageous to use different length linkers to increase the monomer capacity on the molecule.

The accuracy of incorporation by a star molecule is influence by the identity of the linker-fluorophore to which it is attached.

### Nanomaterials

Nanomaterials to the purposes of the inventions set forth herein, are any nanomaterial having multiple sites capable of interacting with biological systems in well controlled manners. The interactions can be covalent, ionic, dipolar, apolar, or a mixture or a combination of such interactions. Such materials have broad application is a number of fields and industries. These nanomaterials exhibit unique properties and functions because of their small size and unique properties.

Exemplary examples of such materials include, without limitation, boron-nitride nanostructures, carbon nanostructures, dendrimers, oligomers and polymers with multiple functional groups, metal oxide nanostructures (e.g., FeO, SiO₂, Al₂O₃, TiO2, ZnO, aluminosilicates, silicoaluminates, quantum dots (e.g., CdSe), etc.), metal clusters (e.g., non-transition metals, transition metals, actinide metals, lanthanide metals, etc. or mixed metal clusters), nanoshells (e.g., metal coated dielectric nanoparticles, metal coated metal nanoparticles, etc.), liposomes, or mixtures of combinations thereof.

### Carbon Nanostructures

Carbon nanostructures are compounds prepared from pure carbon sources (e.g., graphite and diamond), generally under partial oxidizing environment. Certain carbon nanostructures are based on fullerene molecules which are closed and convex cage molecules containing only hexagonal and pentagonal faces. Examples of carbon nanostructures include, without limitation, Buckyballs (Buck Minster fullerenes), nanotubes (e.g., single or multiple walled carbon nanotubes), nanowires, nanowhiskers, or the like, or mixtures of combinations thereof.

### Carbon Nanotubes and Buckyballs

Carbon nanotubes are basically elongated fullerenes resembling graphite sheets wrapped into cylinders and having very high length to width ratios (few nm in diameter and up to 1 mm in length).

Buckyballs are spherical fullerenes (e.g., C60 is most stable and symmetrical and resembles a soccer ball).

Some properties of carbon nanostructures include high tensile strength, physically stable, chemically reactive permitting functionalized nanostructures, doped, hydrophilic functionalized nanostructures, superconducting properties, and optical properties (endohedral fullerenes).

### Dendrimers

Dendrimers are spherical polymeric molecules including a series of chemical shells built on a small core molecule (each shell is called a generation). For example, some dendrimers are made from a core and alternating layers of 2 monomers: acrylic acid and diamine. Dendrimers have a molecular structure in the form of a tree with many branches. Dendrimers can serve as nano-devices for delivery of therapeutics or monomers needed for polymer synthetic, e.g., nucleotide.

### Other Star Molecule Applications

The preparation of Ni-fluorophore-Ni structures.

The preparation of fluorescent tags bound by his-tagged proteins.

The preparation of A-P-toxin/chromophore-P-A. A molecule that could target a cell for death or indicate phosphatase activity. Monitor enzyme activity, i.e., PDE could cleave this molecule.

The detection of the fate and role of dinucleotide phosphate A-PPP-DetectableMoiety-PPP-A or G-PPP-DetectableMoiety-PPP-G can be followed within a cell. Monitor phosphatase activity.

A delivery vehicle containing a receptor ligand and therapeutic attached to a star molecule. Preferable there are multiple copies of the therapeutic are included in the molecule, creating an apparent high local concentration of the therapeutic. An example of this concerns HIV and the delivery vehicle comprises a GP120 protein attached to a dendrimer-type star molecule that also contains multiple copies of a nucleotide therapeutic, such as AZT, attached via linkers to either a detectable or non-detectable core. If the therapeutic that is incorporated by the viral polymerase is excised, another therapeutic may be incorporated due to the presence of additional therapeutics on the proximate dendrimer.

The star molecule may be derivatized to contain primers which may be annealed to template DNA strands. The primers may be either the same or different, depending on the experiment. In a preferred embodiment, the same primer is attached via either a chemical- or a photo-labile linkage, the primers are annealed to template DNA and extended. Subsequently, the template strands are removed (e.g., via heat denaturation) and the primer strands may be isolated from the star via breaking the bond at the 5′ side of the primer. The extended primer strand and the isolated template strands may be used in single molecule sequencing reactions.

Base-labeled and gamma-labeled dNTPs may be used. The labels on the base or the gamma-phosphate may be fluorophores with different spectral characteristics (i.e., donor and acceptors), or they may be a fluorophore and a quencher molecule or a quencher and a fluorophore, respectively. The presence of a detectable label on the base enables tracking of the nascent DNA strand. In the context of star molecules containing a quencher-fluorophore combination, the presence of the quencher reduces fluorescence from the fluorophore until they are separated from each other. This may occur when the fluorescently base-labeled dNTP is incorporated into a nascent DNA strand, enabling monitoring of the nascent strand. Alternatively, this may occur when the quenched, base-labeled dNTP is incorporated into a nascent DNA strand, enabling monitoring of the released star molecule. FRET between the molecule attached to the gamma phosphate and the molecule attached to the base may occur and be monitored. Further, this may involve either changes in fluorescence or changes in quenching efficiency.

Also, because the donor and acceptor are within the star molecule and the polymerase is not labeled, it doesn't require the molecule to be like a flower (i.e., asymmetrical). Originally the inventors thought that an asymmetric molecule would support constant orientation of entry into the active site and the resulting signal would be consistent. The inventors were concerned that a symmetrical donor-acceptor molecule would produce different types of signals depending on the orientation that entered the polymerase active site—having in my mind that the donor was on the pol (hard to leave that thought). This concern was not borne out experimentally, if the donor is adjacent to the acceptor and within the star. This distance, and therefore signal, remains pretty constant. This could involve a nucleoside-PPP-linker-donor-linker-acceptor-linker-PPP-nucleoside or nucleoside-PPP-linker-acceptor-linker-donor-linker-PPP-nucleoside molecule. As before, it is the consistent location of signal and fluorophore features while at that location that provides DNA sequence information.

For details on sequencing reactions for which the compositions of this invention can be used, the reader is referred to the following United States patents and patent applications: U.S. Pat. Nos. 6,982,146, 7,033,764, Ser. Nos. 09/901,782, 10/007,621, 11/007,794, or 11/671,956, incorporated herein by reference. Star molecules are relevant to any sequencing technology as it is easier and more efficient to remove star dNTPs, relative to traditional (monomer) dNTPs.

### Suitable Reagents

Suitable biomolecules include, without limitation, any biomolecule including a phosphate group, a synthetic phosphate replacement moiety or group or any other group capable of displacing a leaving group of a modifying agent designed so that the leaving group is displaceable by a phosphate group or synthetic phosphate replacement moiety or group. Exemplary biomolecules include nucleotides, phosphorylated polypeptides, phosphorylated proteins, phosphorylated sugars or sacchrides, phosphorylated carbohydrates, phosphorylated enzymes, phosphorylated membranes, phosphorylated cells, phosphorylated tissues, phospholipids or any other bio-material or organized structure bearing at least one phosphate group or mixtures or combinations thereof.

Suitable modifying agents include, without limitation, any molecule having a leaving group capable of being displaced by a phosphate group attached to a biomolecule under displacement reaction conditions sufficient to form a phosphate modified biomolecule.

Suitable leaving groups include, without limitation, any leaving group capable of being displaced in a substitution reaction with a phosphoylated biomolecule or a biomolecular including a phosphate group, a phosphate group analog or a phosphate group equivalent. Exemplary group include carbylsulfonates, where the carbyl group is a group including at least one carbon atom and sufficient hydrogen atoms to satisfy the valence state of the group. The group can have one or all of its hydrogen substituted with monovalent atoms such as F, Cl, Br, or I and the carbon atom or atoms can be substituted by certain hetero atoms such as B, C, Si, Ge, N, P, As, O, S, Se, or the like. Exemplary examples of leaving groups include, without limitation, sulfonate groups, halogens, or the like. Exemplary examples of sulfonates include alkylsulfonates, arylsulfonates, alkarylsulfonates, or aralkylsulfonates, where the alkyl, aryl, alkaryl and aralkyl groups include from 1 to about 40 carbon atoms, one or more of which can be a hetero atom such as B, C, Si, Ge, N, P, As, O, S, and/or Se, and including sufficient hydrogen atoms to satisfy the valency, where one or more hydrogen atom can be F, Cl, Br, I, OR, SR, COR, COOR, CONH₂, CONHR, CONRR′, or any other group inert under the substitution/displacement reaction conditions, such as mesylate, ethylsulfonate, tosylate, etc.

Suitable solvents include, without limitation, formamide, dimethylformamide (DMF), n-methylpyrrolidone, acetonitrile, dimethylsulfoxide (DMSO), halogenated solvents such as dichloromethane (DCM), chloroform, carbon tetrachloride, tri-chloroethylene, di-chloroethane, tri-chloroethane, chlorobenzene, or the like, ethers such as furan, tetrahydrofuran, or the like, acetates such as ethyl acetate or the like, ketone such as acetone, methylethylketone (MEK) or the like or other solvent that support or promote S_{N}1 or S_{N}2 substitution reactions.

In certain embodiments, the modifying agent is a linking group including a leaving group capable of being displaced by the terminal phosphate group of the nucleotide, nucleotide polyphosphate or analogs thereof under displacement reaction conditions. The linking group can also include a protected group, such as a protected OH group, a protected NH₂ group, a protected NRH group, a protected NRR′ group, a protected SH group, a protected silicon containing group, a protected boron containing group, a protected phosphorus containing group, or any other group capable of being protected and de-protected for use in subsequence syntheses. In certain embodiments, the reactive groups are used, after de-protection, as attachment sites for tags or labels.

Suitable linkers or linking groups L, L′ and L″ include, without limitation, Q-E-R-E′-Q′, where Q is a leaving group, E and E′ are B, C, Si, Ge, N. P. As, O, S, and/or Se atom-containing moieties, Q′ is a leaving group or a protecting or blocking group, and R is an alkenyl group, an arenyl group, an aralkenyl group and/or a alkarenyl group. Exemplary alkenyl group include, without limitation, saturated orunsaturated, linear, branched or cyclic groups, e.g., —(CH₂)ₙ—, where n is an integer having a value between 1 and 40. Exemplary arenyl group include, without limitation, —(CH₂)ₖ-Ph-(CH₂)ₗ—, where Ph is phenyl and k and l are integers having values between 0 and 20 and where the substituents form a 1,2 (ortho), 1,3 (meta) or 1,4(para) aromatic substitution pattern. Exemplary and non-limiting examples of linkers are shown below:

TABLE 1
Linker Used in the Various Preparatory Methods

| Linker | Structure |
|---|---|
| 1 | 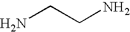 |
| 2 | 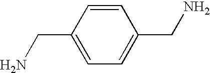 |
| 3 | 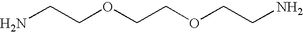 |
| 4 | 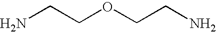 |
| 5 | 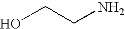 |
| 6 | 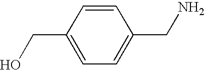 |
| 7 | 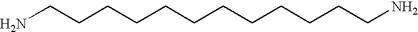 |
| 8 | 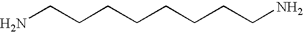 |
| 9 | 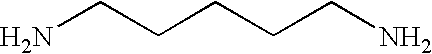 |
| 10 | 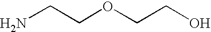 |
| 11 | 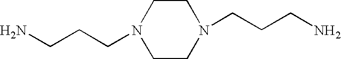 |
| 12 | 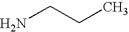 |
| 13 | 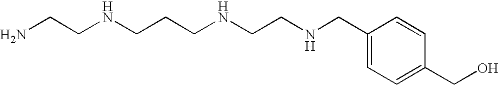 |
| 14 | 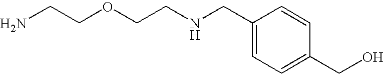 |
| 15 | 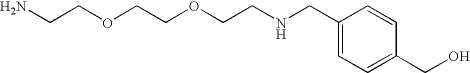 |

Suitable groups having a detectable property, detectable groups, tags or labels including, without limitation, any atom, molecule, atom cluster, nano-particle, nano-structure, quantum dots, or the like capable of reacting with a reactive group on the modified phosphorlated biomolecules to form a covalently or ionically bond therewith. Suitable tags or labels also include, without limitation, any group imparts an unique characteristic to the biomolecule such as a group that is analytically detectable, a group that alters reactively of the biomolecule, a group that permits a desired subsequence chemical modification, a group that permits a desired enzymatic modification, a group that permits enzymatic incorporation of all or a part of the modified biomolecule, a group that acts as a reporter group, or any other group that uniquely affects that properties of the biomolecule. Exemplary examples of groups that are analytically detectable include, without limitation, (1) groups including nmr active atoms such as D(²H), T (³H), ¹³C, ¹⁵N, ¹⁹F, ²⁹Si, ³¹P, ³³S, nmr active metal nuclei, or other nmr active halogen nuclei, (2) far IR, IR or near IR active groups or groups including far IR, IR or near IR active moieties, (3) UV or far UV active group or groups including an UV active moiety, (4) fluorescently active groups or groups including a fluorescently active moiety, (5) phosphorescently active groups or groups including a phosphorescently active moiety, (6) groups capable of undergoing light or chemically induced luminescence or including a moiety capable of undergoing light or chemically induced luminescence, (7) X-ray active groups or groups including an X-ray active moiety, (8) Raman active groups or groups including a Raman active moiety, (9) CD (circular dichroism) active groups or group including CD active groups, (10) neutron activation active groups or groups including a neutron activation active moiety, (11) paramagnetically active groups or groups including a paramagnetically active moiety, or (12) any other group have an analytically detectable property.

Suitable phosphate-containing groups, Z, include, without limitation, a mono phosphate group, —OP(O)(OA)O—, or a polyphosphate group, PₓO_{y}A_{z}, where x, y and z are an integers having values consistent with a given polyphosphate group, where P is phosphorus, O is oxygen and A is an atom, ion, or group, x is an integer having a value ranging from 1 to 10 or more and y is an integer having a value equal to x+3 and z is an integer having a value equal to x (e.g., P₂O₇A₂-OP(O)(OA)OP(O)(OA)O—, P₃O₁₀A₃-OP(O)(OA)OP(O)(OA)OP(O)(OA)O—, etc.). Sometimes the phosphate-containing groups are represented as Pᵢ, where P represent a phosphate moiety and I in an integer having a value from 1 to 10 or more. In all of the phosphate groups or moieties set forth above, one more of the oxygen atoms can be replaced by a B, C, Si, Ge, N. P. As, O, S, or Se atoms or atom containing groups or moieties. In all of the phosphate groups or moieties, A is a hydrogen atom or ion, an alkali atom or ion, an ammonium ion (R¹R²R³R⁴N⁺), a phosphonium ion (R¹R²R³R⁴P⁺), an R group, other metal atoms or ions, or mixtures or combinations thereof, where R, R¹, R², R³, and R⁴ are the same or different and are carbyl group having between 1 and 20 carbon atoms, where one more of the carbon atoms can be replaced by a B, C, Si, Ge, N. P. As, O, S, or Se atoms or atom containing groups or moieties or groups and where one or more hydrogen atoms can be replaced with F, Cl, Br, I, OR, SR, COR, COOR, CONH₂, CONHR, CONRR′, or any other monovalent group inert or substantially inert under the substitution/displacement reaction conditions.

Suitable quencher or quenching group include, without limitation, DDQ-I, Dabcyl (4-(4-dimethylamino-phenylazo)-benzene), Eclipse, Iowa Black FQ, BHQ-1, QSY-7, BHQ-2, DDQ-II, Iowa Black RQ, QSY-21, BHQ-3, ATTO 540Q, ATTO 580Q, ATTO 612Q and mixtures or combinations thereof.

Suitable molecular cores for formation of star molecules include, without limitation, bifunctional molecules, polyfunctional molecules, polyfunctional boron-nitride nanostructures, polyfunctional carbon nanostructures, polyfunctional dendrimers, polyfunctional oligomers, polyfunctional polymers, polyfunctional metal oxide nanostructures (e.g., FeO, SiO₂, Al₂O₃, TiO2, ZnO, aluminosilicates, silicoaluminates, etc.), polyfunctional quantum dots (e.g., CdSe, etc.), polyfunctional metal clusters (e.g., non-transition metals, transition metals, actinide metals, lanthanide metals, etc. or mixed metal clusters), polyfunctional nanoshells (e.g., metal coated dielectric nanoparticles, metal coated metal nanoparticles, etc.), polyfunctional liposomes, or any other structure that can support attachment of a plurality of nucleotides through their terminal phosphate or mixtures of combinations thereof.

### Synthetic Schemes of the Invention

One embodiment of a method of the present invention is Scheme I shown below:

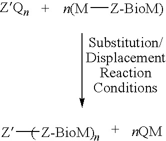

where: (a) Z′ is a group having a detectable property or a carbyl group having from 1 to 40 carbon, where one or more of the carbon atoms can be replaced with a hetero atoms selected from the group consisting of B, C, Si, Ge, N, P, As, O, S, or Se and having sufficient hydrogen atoms to satisfy the valency of the carbyl group, where one or more hydrogen atoms can be replaced with F, Cl, Br, I, OR, SR, COR, COOR, CONH₂, CONHR, CONRR′, or any other monovalent group inert or substantially inert under the substitution/displacement reaction conditions; (b) Q is a leaving group such as Ms (mesylate), Ts (tosylate), Cl, Br, I, Tf, or the like; (c) M is a monovalent ion such as H⁺, Li⁺, Nan, K⁺, Rb⁺, Cs⁺, Cu¹⁺, H₄N⁺, H₂R₂N, HR₃N⁺, R₄N⁺, H₄P⁺, H₂R₂P⁺, HR₃P⁺, R₄P⁺ or the like; (d) Z is a phosphate-containing group having one or a plurality of phosphate moieties (—OP(O)(OH)O—) or analogs thereof, (e) BioM is a biomolecule such as a base, a nucleoside, nucleotide, oligonucleotide, nucleic acid, amino acid, organic soluble polypeptide, organic soluble proteins, saccharide or sugar, organic soluble carbohydrate, a lipid, derivatives and analogs thereof, a compound including one or more of the afore listed biomolecules compounds, or the like; (f) n is an integer having a value between 1 and 10. In Scheme I, a linker or linking group can be inserted between Z and Z′ and/or between Z and BioM.

From the above example, some of the features of this method can be seen as follows: (1) Scheme I avoids activation of phosphates and P—O—P bond formation, (2) Scheme I avoids side-products as the phosphate is blocked at one end, (3) Scheme I is run under mild reaction conditions and easy to manage synthetically, and (4) Scheme I permits the modifying agent Q Z′ to be used in excess to increase consumption of expensive biomolecules such as nucleotides.

Another embodiment of the method of the present invention is Scheme II shown below:

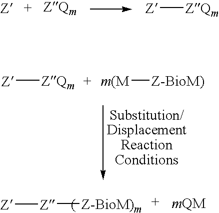

where: (a) Z′ is a group having a detectable property or a carbyl group having from 1 to 40 carbon, where one or more of the carbon atoms can be replaced with a hetero atoms selected from the group consisting of B, C, Si, Ge, N, P, As, O, S, or Se and having sufficient hydrogen atoms to satisfy the valency of the carbyl group, where one or more hydrogen atoms can be replaced with F, Cl, Br, I, OR, SR, COR, COOR, CONH₂, CONHR, CONRR′, or any other monovalent group inert or substantially inert under the substitution/displacement reaction conditions; (b) Z″ is a multi-functional group capable of reacting with up to m phosphorylated biomolecules to form star molecules having 1 to 1000 arms; (c) Q is a leaving group such as Ms (mesylate), Ts (tosylate), Cl, Br, I, Tf, or the like; (d) M is a monovalent ion such as H⁺, Li⁺, Na⁺, K⁺, Rb⁺, Cs⁺, Cu¹⁺, H₄N⁺, H₂R₂N⁺, HR₃N⁺, R₄N⁺, H₄P⁺, HR₂P⁺, HR₃P⁺, R₄P⁺ or the like; (e) Z is a phosphate-containing group having one or a plurality of phosphate moieties (—OP(O)(OH)O—) or analogs thereof, (f) BioM is a biomolecule such as a base, a nucleoside, nucleotide, oligonucleotide, nucleic acid, amino acid, organic soluble polypeptide, organic soluble proteins, saccharide or sugar, organic soluble carbohydrate, a lipid, derivatives and analogs thereof, a compound including one or more of the afore listed biomolecules compounds, or the like; and (g) m is an integer having a value between 1 and 1000. In Scheme II, a linker or linking group can be inserted between Z′ and Z″, Z″ and Z and/or between Z and BioM.

Another embodiment of the method of the present invention is as Scheme III is shown below:

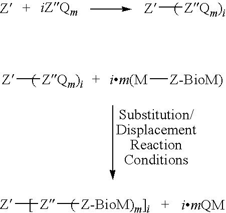

where: (a) Z′ is a group having a detectable property or a carbyl group having from 1 to 40 carbon, where one or more of the carbon atoms can be replaced with a hetero atoms selected from the group consisting of B, C, Si, Ge, N, P, As, O, S, or Se and having sufficient hydrogen atoms to satisfy the valency of the carbyl group, where one or more hydrogen atoms can be replaced with F, Cl, Br, I, OR, SR, COR, COOR, CONH₂, CONHR, CONRR′, or any other monovalent group inert or substantially inert under the substitution/displacement reaction conditions; (b) Z″ is a multi-functional group capable of reacting with up to m phosphorylated biomolecules to form star molecules having 1 to 1000 arms; (c) Q is a leaving group such as Ms (mesylate), Ts (tosylate), Cl, Br, I, Tf, or the like; (d) M is a monovalent ion such as H⁺, Li⁺, Na⁺, K⁺, Rb⁺, Cs⁺, Cu¹⁺, H₄N⁺, H₂R₂N⁺, HR₃N⁺, R₄N⁺, H₄P⁺, H₂R₂P⁺, HR₃P⁺, R₄P⁺ or the like; (e) Z is a phosphate-containing group including one or a plurality of phosphate moieties (—OP(O)(OH)O—) or analogs thereof, (f) BioM is a biomolecule such as a base, a nucleoside, nucleotide, oligonucleotide, nucleic acid, amino acid, organic soluble polypeptide, organic soluble proteins, saccharide or sugar, organic soluble carbohydrate, a lipid, derivatives and analogs thereof, a compound including one or more of the afore listed biomolecules compounds, or the like; (g) m is an integer having a value between 1 and 1000; and (h) i is an integer having a value between 1 and 1000. In Scheme III, a linker or linking group can be inserted between Z′ and Z″, Z″ and Z and/or between Z and BioM.

These methods are ideally suited for preparing modified biological phosphate esters, e.g., NMP, NDP, phosphorylated carbohydrates, phosphorylated peptides, phospholipids, and biomolecules including a phosphate ester or synthetic equivalent thereof and biomolecules including one or more structures such as sugars, saccharides, nucleoside bases, nucleoside, NMP, NDP, NTP, carbohydrates, amino acids, polypeptides, proteins, lipids, fatty acids, etc.

### EXPERIMENTS OF THE INVENTION

γ-esters of NTPs have found extensive applications in biotechnology. See Alexandre Lebedev, TriLink BioTechnologies Technical Information: Enzymatic activity of selected nucleoside 5′-Triphosphates and their analogs for more details. However, only a few methods have been developed to prepare these molecules. Typically a monophosphate ester is reacted with an activating reagent and the resulting intermediate is quenched by a diphosphate. See Natalia Nikolajewna Gaiko, Dissertation 2001, Synthesis and analysis of fluorescently labeled deoxynucleotides en route single molecule DNA-sequencing Johann Wolfgang Goethe-University, Frankfurt am Main, Germany, and references cited therein for more details. While this method has dominated the literature it usually takes long reaction times (>24 hr) to give moderate yields (<50%). A different approach activates NTPs with DCC and the resulting intermediate is quenched by a nucleophile. See D. G. Knorre, V. A. Kurbatov, and V. V. Samukov, FEBES Lett. 1976, 105 for more details. Although this method works extremely well with amines, it barely works for alcohols.

There have been reports using monophosphate, diphosphate and rarely, triphosphate, as nucleophiles to react with a substrate bearing a leaving group. See V. Jo Davisson, Andrew B. Woodside, Timothy R. Neal, Kay E. Stremler, Manfred Muehlbacher, and C. Dale Poulter, J. Org. Chem. 1986, 51, 4768 for more details. See A. M. Shprit, L. O. Kononov, V. I. Torgov, and V. N. Shibaev, Russian Chemical Bulletin, International Edition, 2005, 481 for more details. See Vyas M. Dixit and C. Dale Poulter, Tetrahedron Lett. 1984, 4055 for more details. Notably, linear triphosphate has used as anucleophile to react with Adenosine-5′-tosylate to yield ATP in 55% yield. See V. Jo Davisson, Darrell R. Davis, Vyas M. Dixit, and C. Dale Poulter, J. Org. Chem. 1987, 1794 for more details. Uracil diphosphate (UDP) has used as a nucleophile to react with a-halo-sugars to produce sugar nucleotides in modest yields (10-30%). See Michael Arlt and Ole Hindsgaul, J. Org. Chem. 1995, 14 for more details.

Moreover, ATP and its analogues were selectively alkylated at their gamma-position with 1-(2-nitrophenyl)diazoethane. See J. W. Walker, G. P. Reid, J. A. McCray, and D. R. Trentham, J. Am. Chem. Soc. 1988, 7170. This method demands the preparation of unstable diazo-intermediates in multiple steps and has only been adopted to prepare several caged nucleotides.

Prompted by these literature observations, the inventors set out to investigate a different approach to preparation of γ-esters of NTPs by using the terminal phosphate of an NTP as a nucleophile to attack a substrate which bears a variety of leaving groups. Herein we describe this methodology and extend it to the preparation of a wide variety of phosphate esters.

### Example 1

This example illustrates a general method for preparing phosphate modified biomolecules, with DATP as the biomolecule and Cbz-6-Ms as the modifying agent, where 6 represents linker 6 or 1-hydroxymethyl,2-aminomethylbenzene.

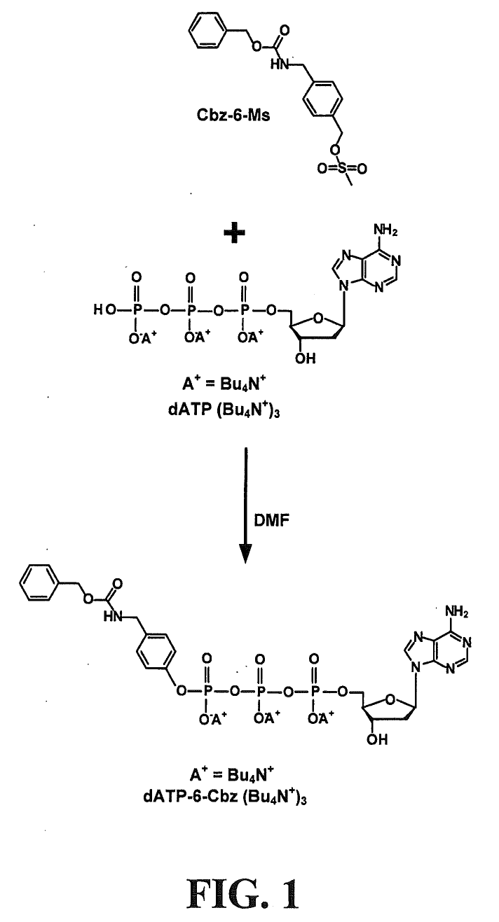

Referring now to FIG. 1, the compound Cbz-6-Ms (20 μmol) was reacted with DATP (Bu₄N⁺) (5 μmol) in DMF (0.4 mL) at room temperature (r.t.) overnight. The reaction mixture was concentrated to dryness on a rotary evaporator and redissolved in water (1 mL). Centrifugation removed the pellet, while the aqueous solution was subjected to HPLC purification (reverse phase or anion exchange) to afford the dATP γ-ester, dATP-6-Cbz. After lyophilization the product was dissolved in water and quantified by UV absorption at 258 nm (1.51 mmol, 30% yield).

From the above example, some of the features of this method can be seen as follows: (1) the method avoids activation of phosphates and P—O—P bond formation; (2) the method avoids side-products with triphosphate being blocked at one end; (3) the method is run under mild reaction conditions and easy to operate; and the reactant R-Q can be used in excess to better consume the expensive NTPs or other phosphorylated biomolecule.

This method can be extended to preparation of other biological relevant phosphate esters: NMP, NDP, phosphorylated carbohydrates, phosphorylated peptides, phospholipids, and combinations of them. It can be generalized as in the following scheme:

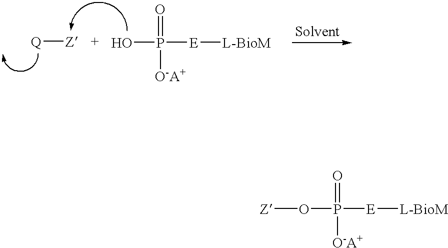

where Q is a leaving group selected from the group consisting of Ms, Ts, Cl, Br, I, Tf, N2+ or the like, Z′ is a group having a desired property such as a detectable property or capable of changing a detectable property of a secondary compound (i.e., capable of quenching a fluorophore), L is a linker, E is a main group element selected from the group consisting of C, N, O, S, Se, As, Si, Ga, Ge, or the like, BioM is a biomolecule selected from the group consisting of a nucleoside or nucleoside analog, a nucleotide or nucleotide analog, an oligonucleotide or oligonucleotide analog, a nucleic acid or a nucleic acid analog, a polypeptide, a protein, a glycopeptide, glycoprotein, an enzyme, a sugar, a saccharide, a polysaccharide, a carbohydrate, a lipid, membrane, a cell, or any other bio-material and A is a monovalent counterion selected from the group consisting of Li⁺, Na⁺, K⁺, Rd⁺, Cs⁺, Cu⁺, R₄N⁺, R₃HN⁺, R₄P⁺, R₃HP⁺, or the like. The solvent is selected from the group consisting of DMF, MeCH, DMSO, DCM, or the like.

As a summary, a new chemistry was developed to prepare nucleotide phospho-ester and was tested with different bases or nucleophiles, using different substrate leaving groups or attacking different types of carbon. The results show that the conditions can be worked out for a wide variety of substrates. Other projects included the synthesis of new linkers derived from Linker 2, the tentative synthesis of a 3′-phosphate 5′dNTP as a terminator, the synthesis of a dual-labeled dNTP, and the synthesis of the star molecule along with primers and dNTP labeling.

In each case the nucleotide reacts with a substrate which bears a good leaving group to give the desired product. The results are summarized below in Table 1.

More specific examples are presented in the following table. Unless noted, all solvents are Aldrich anhydrous quality and all reactants were dried under vacuum at least 4 hr. Cbz-10-Ms represents Cbz-NH—CH₂CH₂OCH₂CH₂O Ms. Some features of the method can be seen from the table:

TABLE 1
List of Phosphate Nucleotide Prepared by the Method of Example 1

| | | | Solvent | | | | Rxn | | ms | |
|---|---|---|---|---|---|---|---|---|---|---|
| | | Amt | Volume | Conc. | | Substrate | time | Yield | (ESI- | |
| # | Nucleotide | μmol | μL | mM | Substrate | eq. | (hr) | (%) | neg) | notes |
| 1 | dATP(Bu₄N⁺)₃ | 5 | 200 | 25 | Cbz-6-Ms | 4.6 | 16 | 33 | 743.6 | SAX purified |
| | | | | | | | | | (M-H) | |
| 2 | dATP(Bu₄N⁺)₃ | 5 | 400 | 12.5 | TFA-6-Ms | 4 | 18 | 30 | 705.4 | SAX purified |
| | | | | | | | | | (M-H) | |
| 3 | dATP(Bu₄N⁺)₃ | 2 | 250 | 8 | Cbz-10-Ms | 9.5 | 20 | 42 | 711.4 | SAX purified |
| | | | | | | | | | (M-H) | |
| 4 | dGTP(Bu4N⁺)₃ | 5 | 200 | 25 | Cbz-10-Ms | 7 | 16 | 25 | 727.2 | SAX purified |
| | | | | | | | | | (M-H) | |
| 5 | dTTP(Bu4N⁺)₃ | 5 | 150 | 33.3 | Cbz-10-Ms | 7 | 13 | 24 | 702.2 | SAX purified |
| | | | | | | | | | (M-H) | |
| 6 | dCTP(Bu4N⁺)₃ | 5 | 150 | 33.3 | Cbz-10-Ms | 7 | 12 | 28 | 687.2 | SAX purified |
| | | | | | | | | | (M-H) | |
| 7 | dUTP(Bu4N⁺)₃ | 3 | 100 | 30 | Cbz-10-Ms | 5.3 | 16 | 18 | 688.4 | SAX purified |
| | | | | | | | | | (M-H) | |
| 8 | ATP(Bu4N⁺)₃ | 5 | 200 | 25 | Cbz-10-Ms | 7 | 16 | 26 | 727.2 | SAX purified |
| | | | | | | | | | (M-H) | |
| 9 | dADP(Bu4N⁺)₂ | 3 | 200 | 15 | Cbz-10-Ms | 5 | 19 | 22 | | not isolated |
| 10 | dADP(Bu4N⁺)₂ | 3 | 400 | 7.5 | Cbz-10-Ms | 5 | 16 | 7 | 631.4 | SAX purified |
| | | | | | | | | | (M-H) | |
| 11 | dATP(Bu₄N⁺)₃ | 2 | 100 | 20 | PhCH2Br | 5 | 14 | 30 | 580.4 | C18 purified |
| | | | | | | | | | (M-H) | |
| 12 | dATP(Bu₄N⁺)₃ | 1 | 100 | 10 | CH2═CHCH2Br | 10 | 14 | 36 | 530.2 | SAX purified |
| | | | | | | | | | (M-H) | |
| 13 | dATP(Bu₄N⁺)₃ | 2 | 100 | 20 | CH≡CCH2-Ts | 5 | 14 | 37 | 528.4 | SAX purified |
| | | | | | | | | | (M-H) | |
| 14 | dATP(Bu4N⁺)₃ | 2 | 100 | 20 | PhCH2CH2Br | 5 | 14 | 14 | | C18 purified |
| 15 | dATP(Bu4N⁺)₃ | 2 | MeCN 250 | 8 | Cbz-10-Ms | 9.5 | 20 | 20 | | SAX analyzed |
| 16 | dATP(Bu4N⁺)₃ | 2 | dioxane 250 | 8 | Cbz-10-Ms | 9.5 | 20 | 18 | | SAX analyzed |
| 17 | dATP(Bu4N⁺)₃ | 2 | MeOH 250 | 8 | Cbz-10-Ms | 9.5 | 20 | 10 | | SAX analyzed |
| 18 | dATP(Bu4N⁺)₃ | 2 | NMP 250 | 8 | Cbz-10-Ms | 9.5 | 20 | 22 | | SAX analyzed |
| 19 | dATP(Bu4N⁺)₃ | 2 | DCE 250 | 8 | Cbz-10-Ms | 9.5 | 18 | 7 | | SAX anal., solv |
| | | | | | | | | | | evap o/n to 50 |
| 20 | dATP(Bu4N⁺)₃ | 2 | diglyme 250 | 8 | Cbz-10-Ms | 9.5 | 20 | 14 | | SAX analyzed |
| 21 | dATP(Bu4N⁺)₃ | 2 | 250 | 8 | Cbz-10-Ms | 9.5 | 20 | 23 | | SAX analyzed, |
| | | | | | | | | | | repeat of #3 |
| 22 | dATP(Bu4N⁺)₃ | 2 | HMPA 250 | 8 | Cbz-10-Ms | 9.5 | 20 | 0 | | SAX analyzed |
| 23 | dATP(Bu4N⁺)₃ | 2 | DMSO 250 | 8 | Cbz-10-Ms | 9.5 | 20 | 20 | | SAX analyzed |
| 24 | dATP(Bu4N⁺)₃ | 2 | i-PrOH 250 | 8 | Cbz-10-Ms | 9.5 | 20 | 18 | | SAX analyzed |
| 25 | dATP(Bu4N⁺)₃ | 2 | 250/H₂O 10 | 8 | Cbz-10-Ms | 9.5 | 20 | 6 | | SAX analyzed |
| 26 | dATP(Bu4N⁺)₃ | 2 | 250/Air | 8 | Cbz-10-Ms | 9.5 | 20 | 11 | | SAX analyzed |
| 27 | dATP(Bu4N⁺)₃ | 2 | 50 | 40 | Cbz-10-Ms | 9.5 | 20 | 15 | | SAX analyzed |
| 28 | dATP(Bu4N⁺)₃ | 2 | 100 | 20 | Cbz-10-Ms | 9.5 | 20 | 25 | | SAX analyzed |
| 29 | dGTPαB(Bu4N⁺)₃ | 7.3 | 40 | 183 | TFA-6-Ms | 4 | 20 | 10 | | SAX purified, α- |
| | | | | | | | | | | Borano-dGTP |
| 30 | dATP(Bu4N⁺)₃ | 2 | HMPA 250 | 8 | Cbz-10-Ms | 9.5 | 20 | 0 | | SAX analyzed, |
| | | | | | | | | | | repeat of #22 |
| 31 | dUTP(Bu4N⁺)₄ | 3 | 100 | 30 | Cbz-10-Ms | 5.3 | 16 | 17.7 | 909.4, | SAX, |
| | | | | | | | | | 1130.4 | doubly&triply |
| | | | | | | | | | | modified |

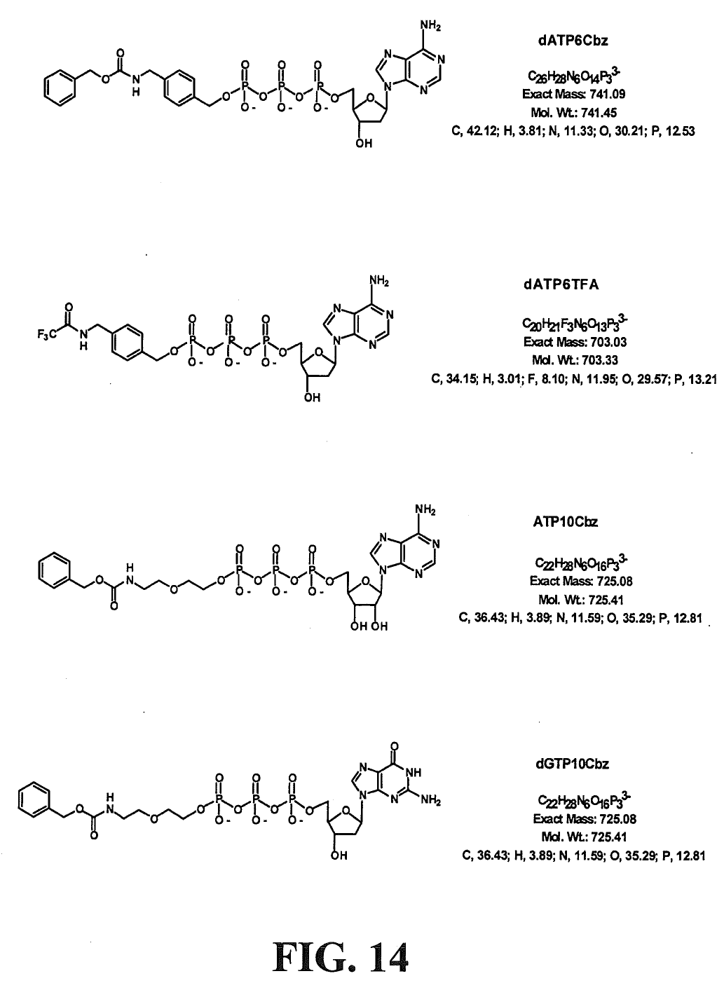

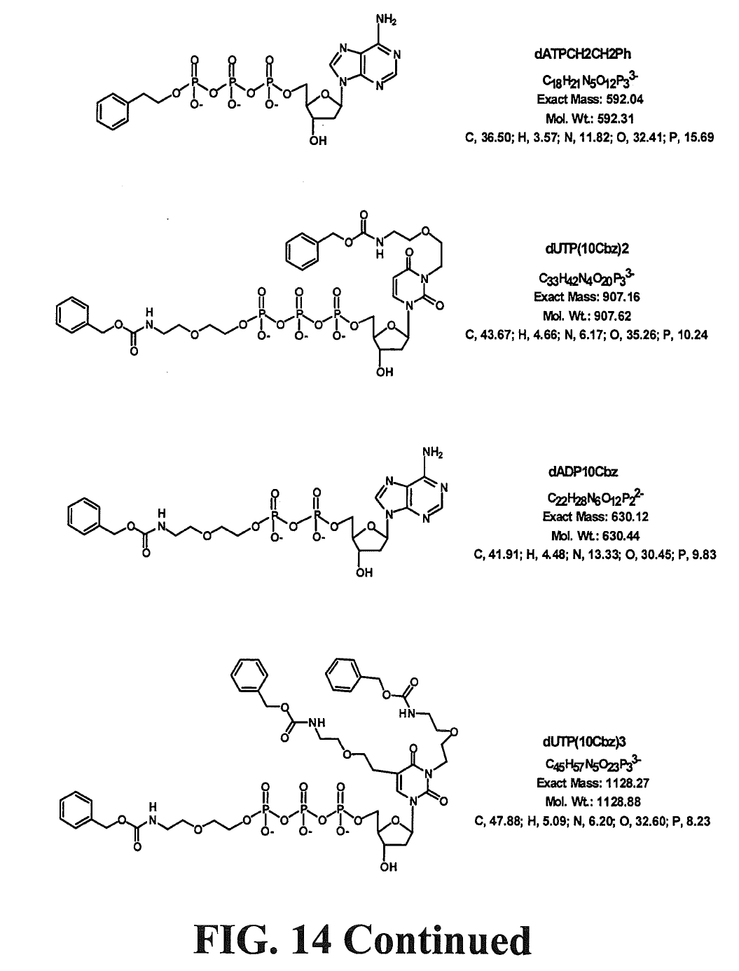

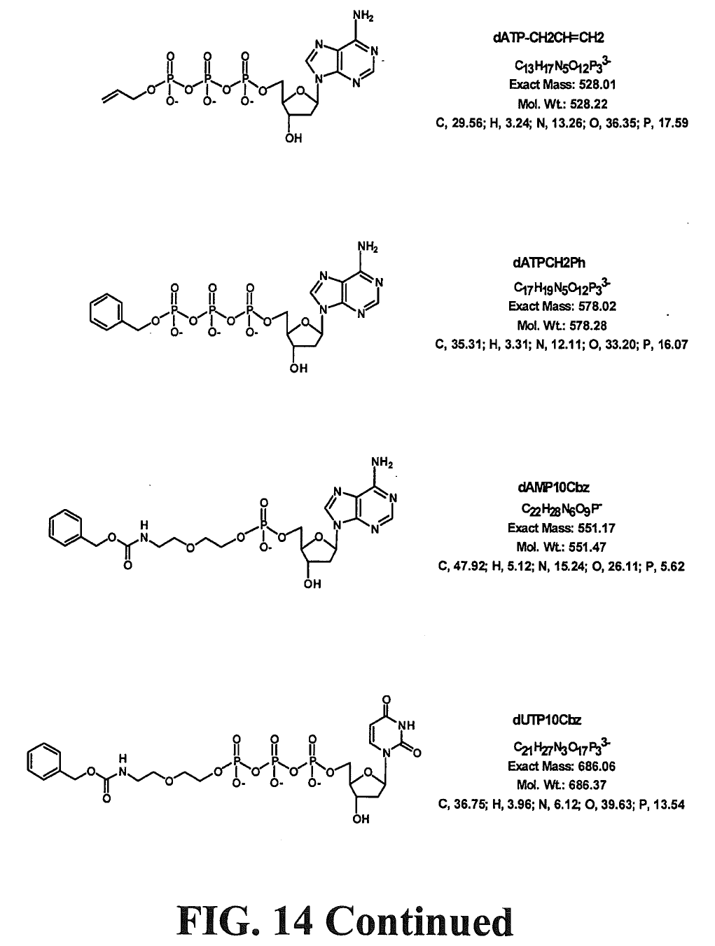

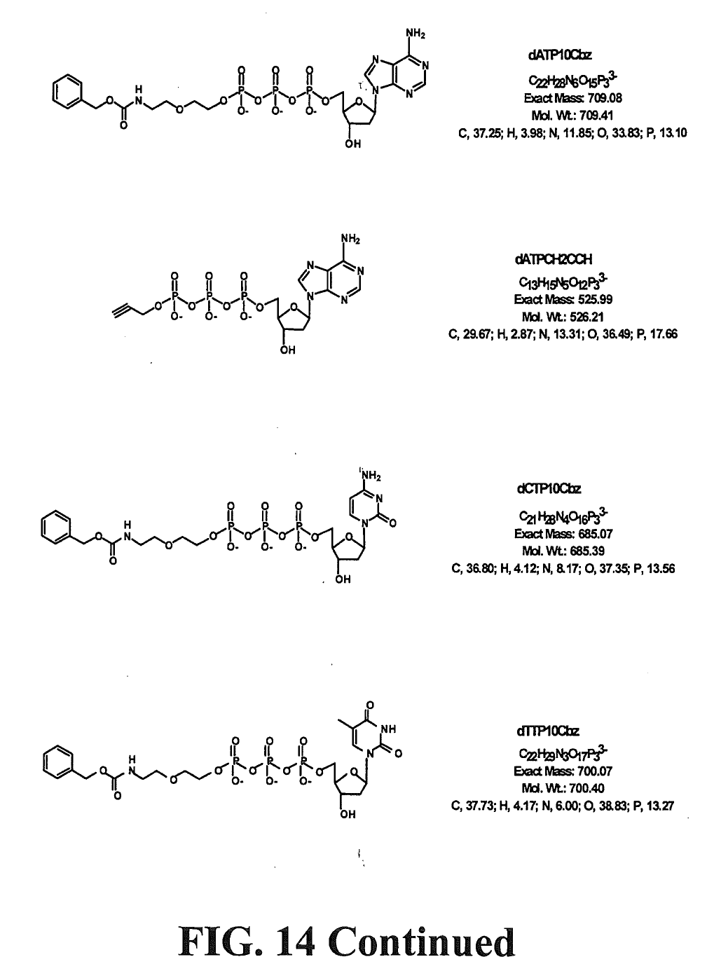

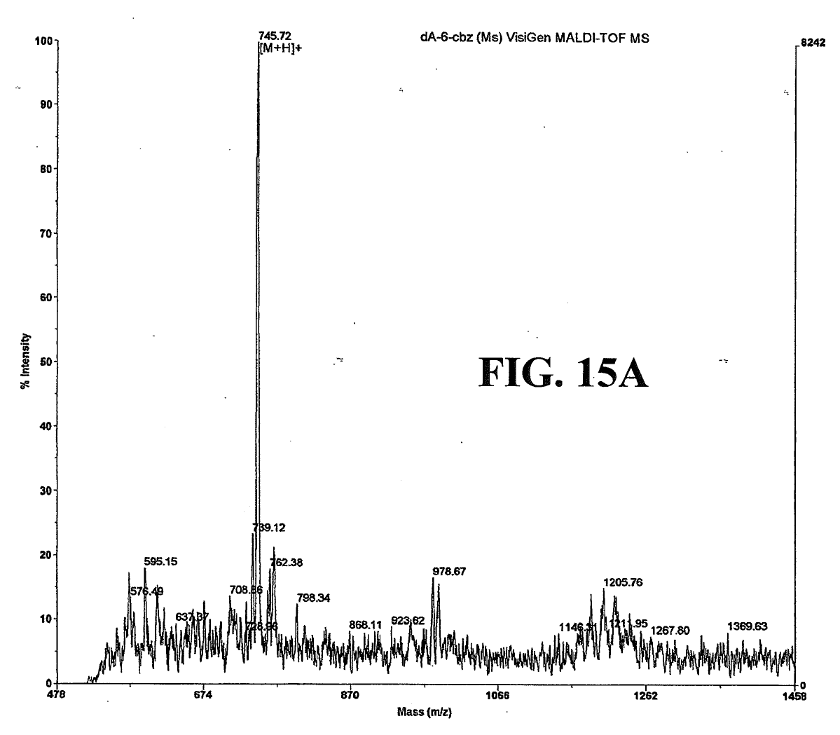

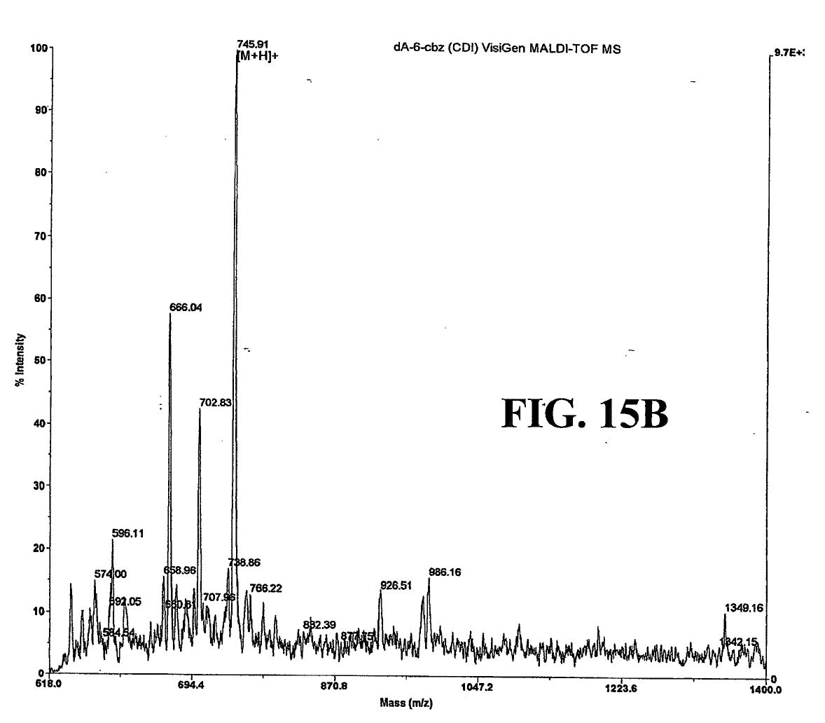

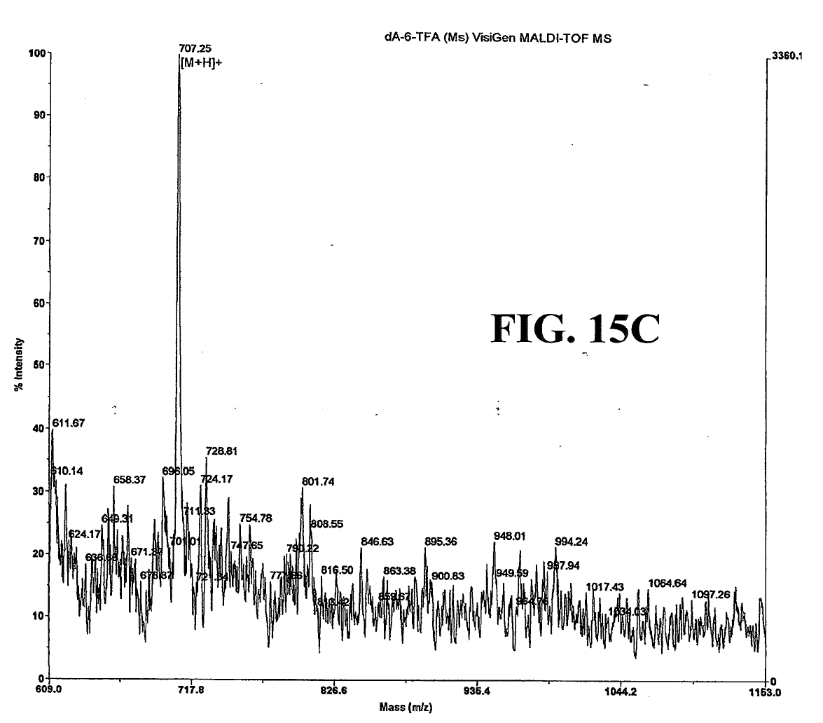

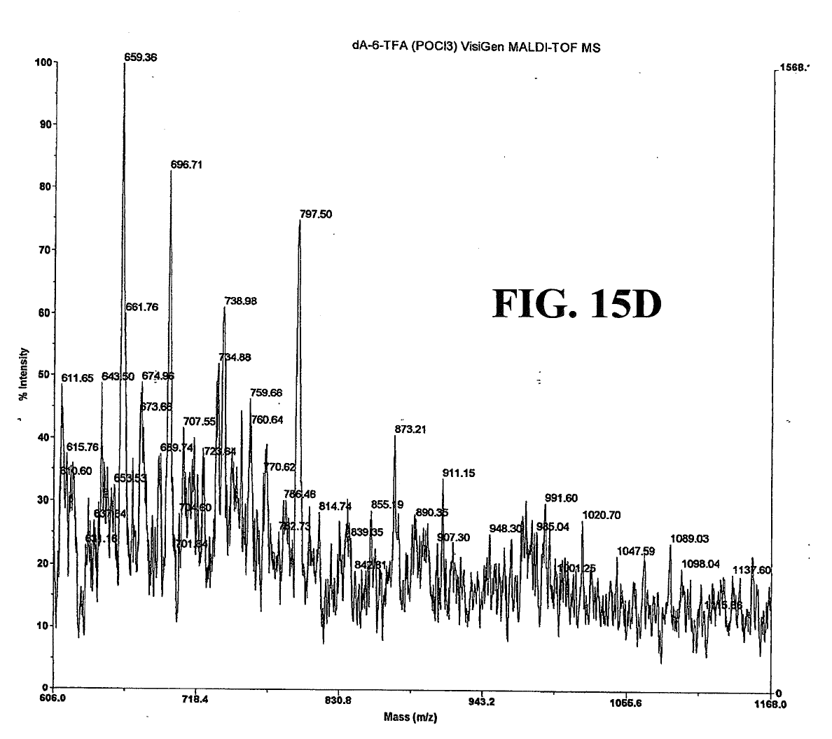

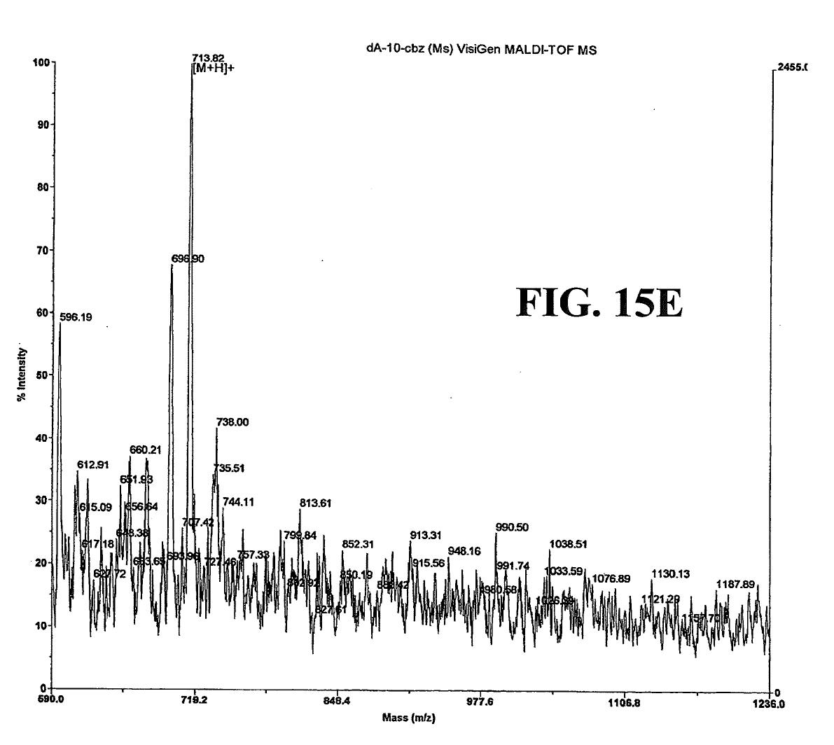

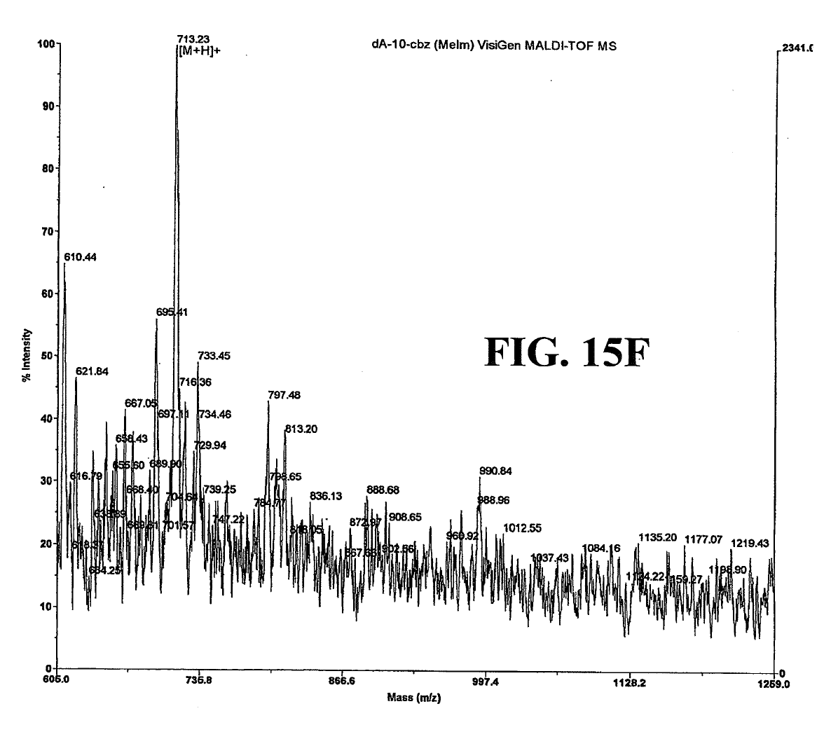

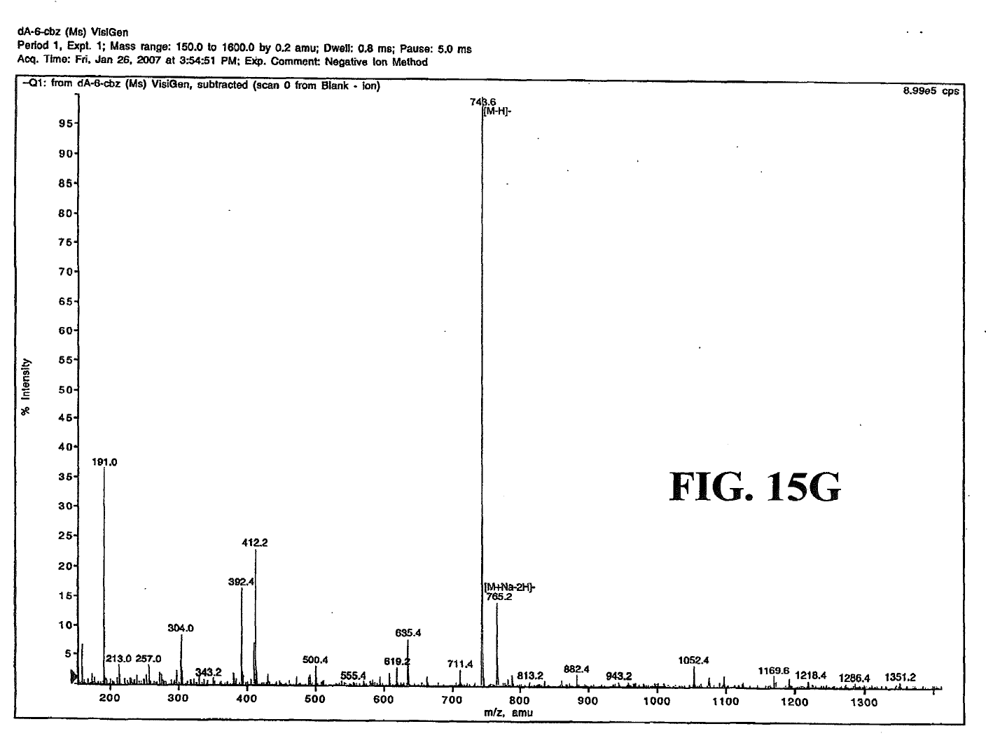

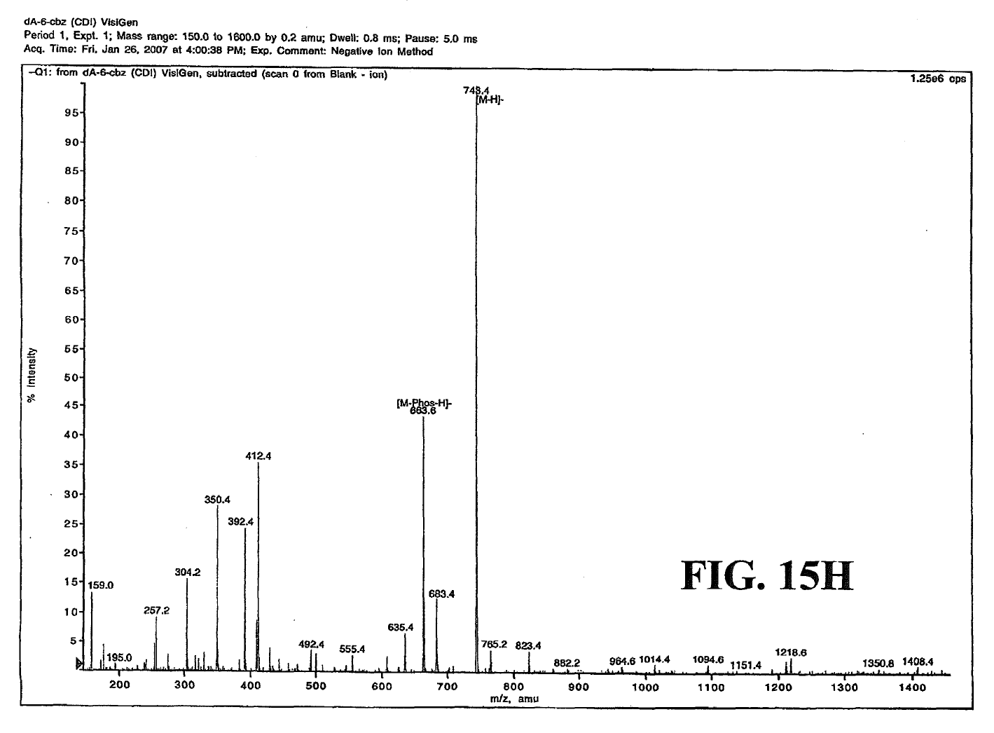

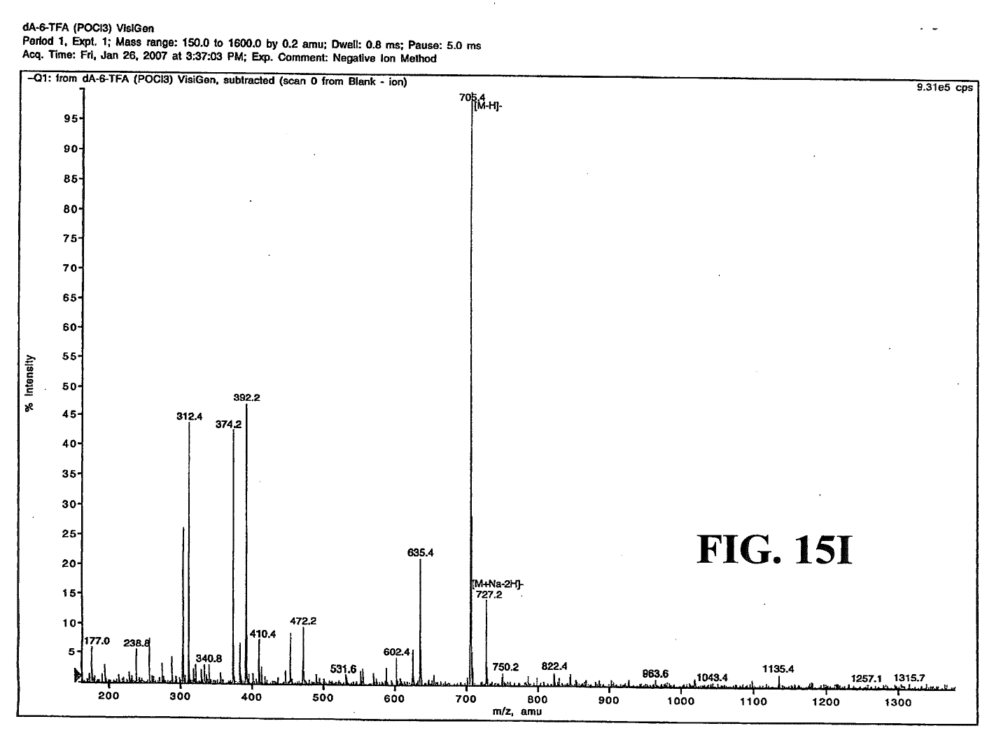

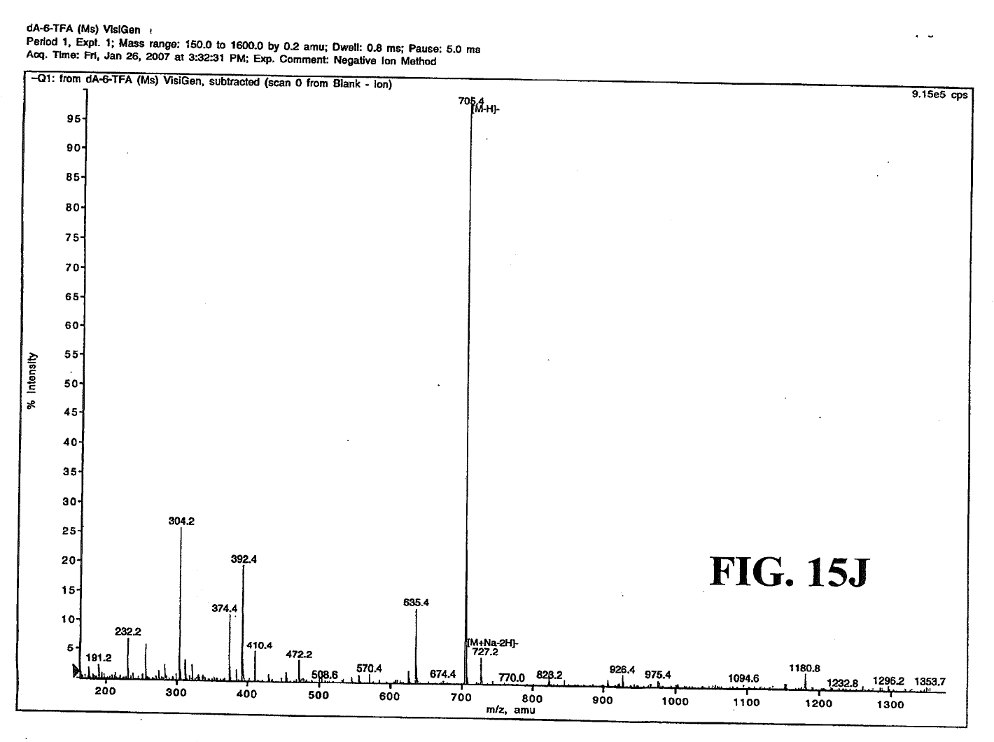

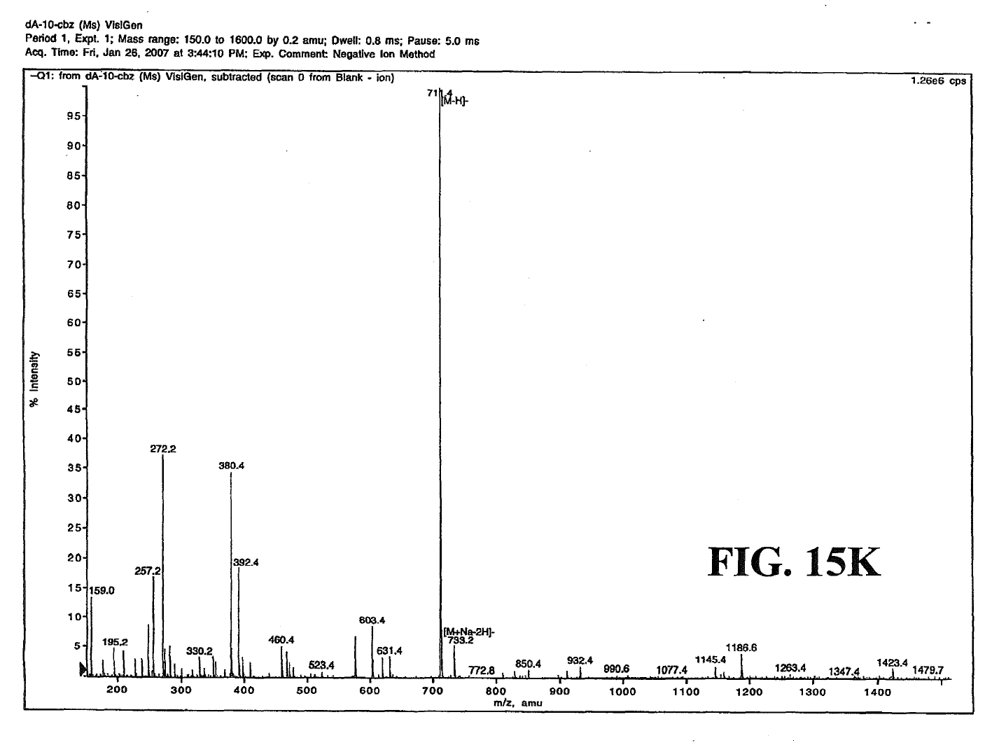

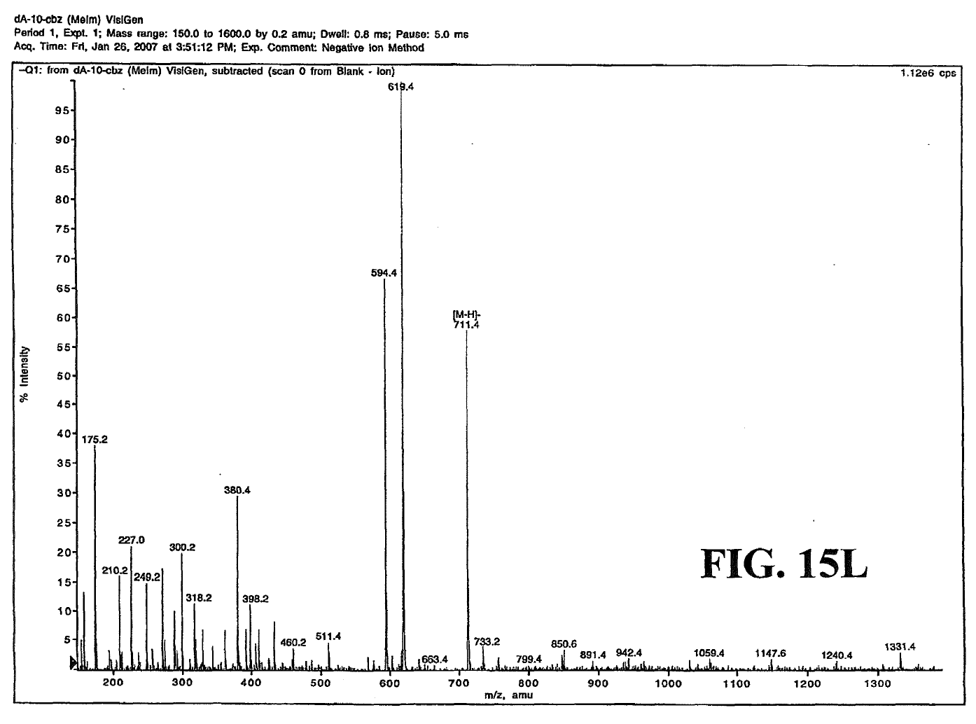

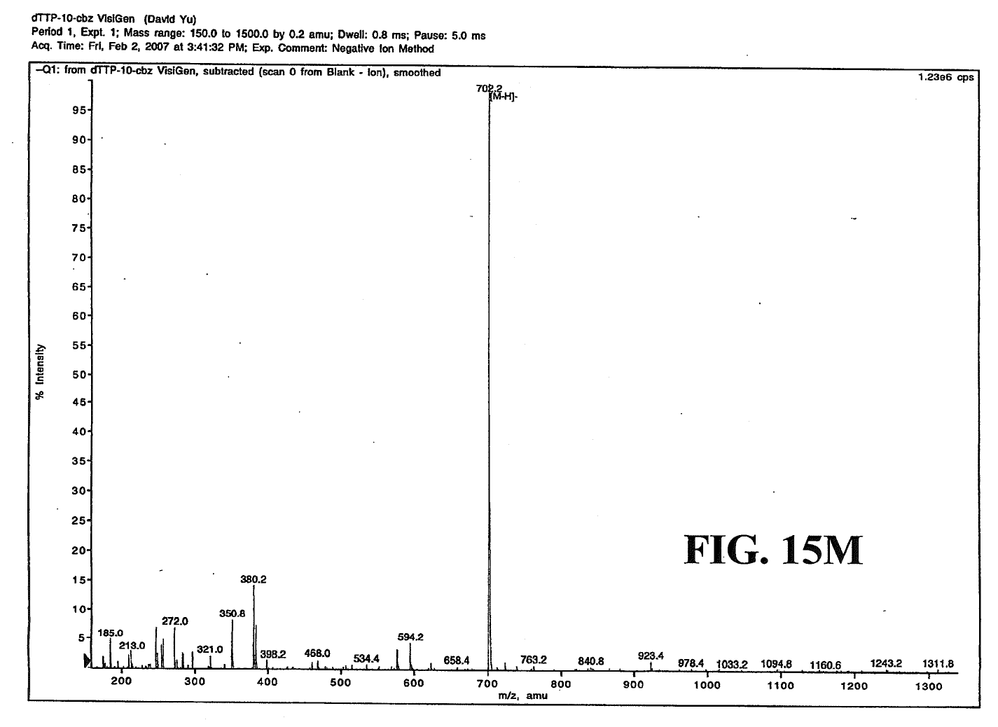

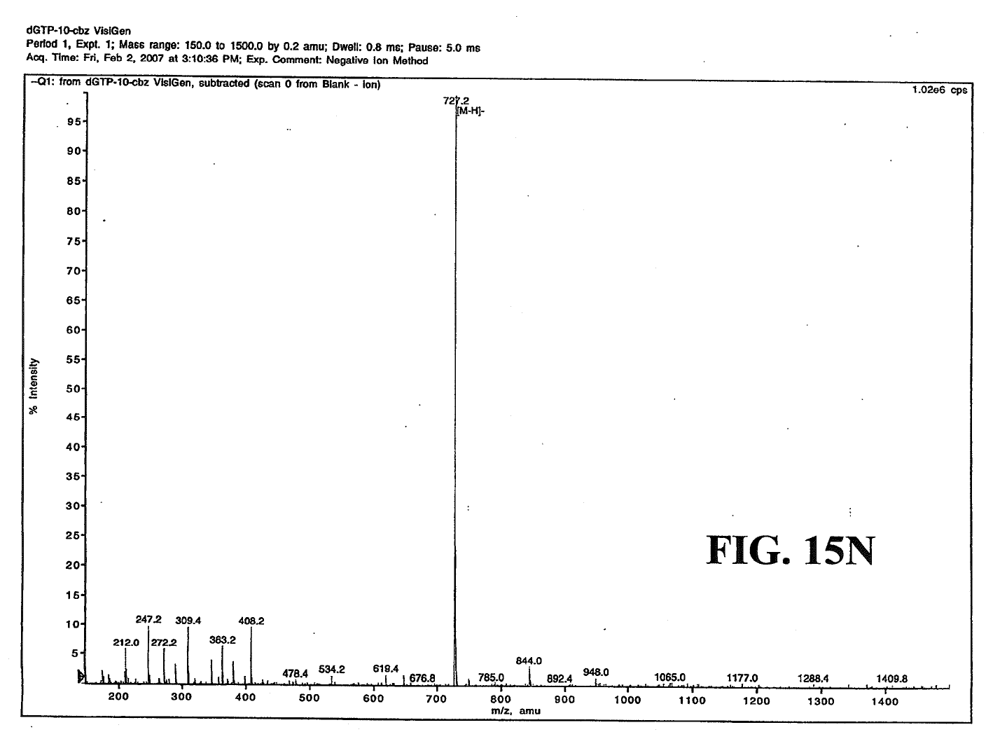

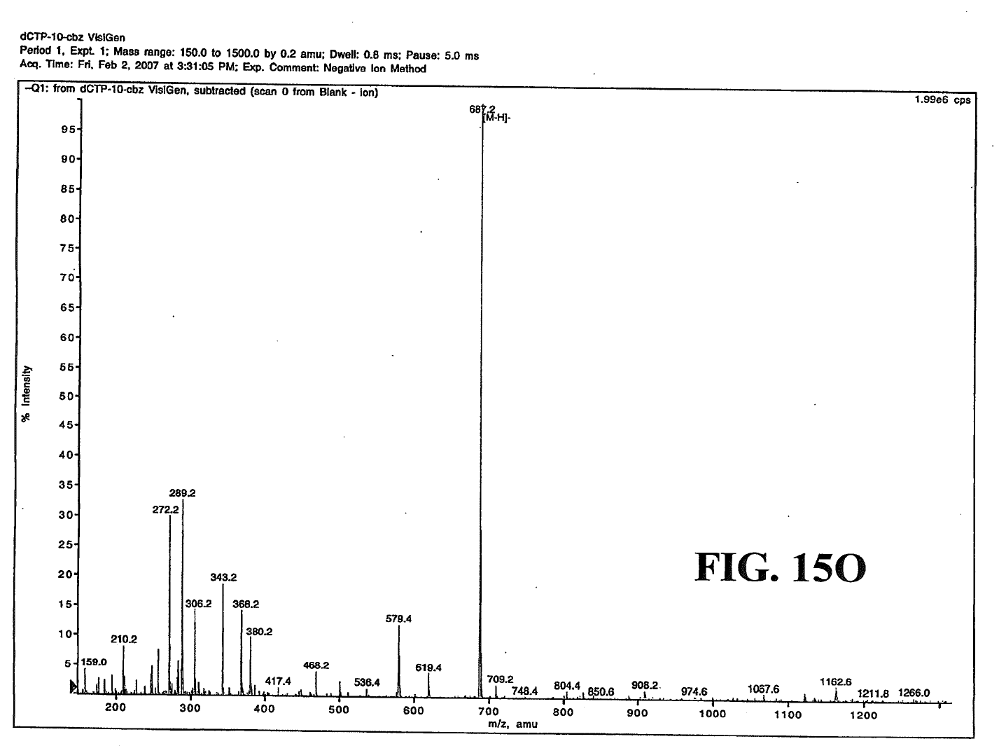

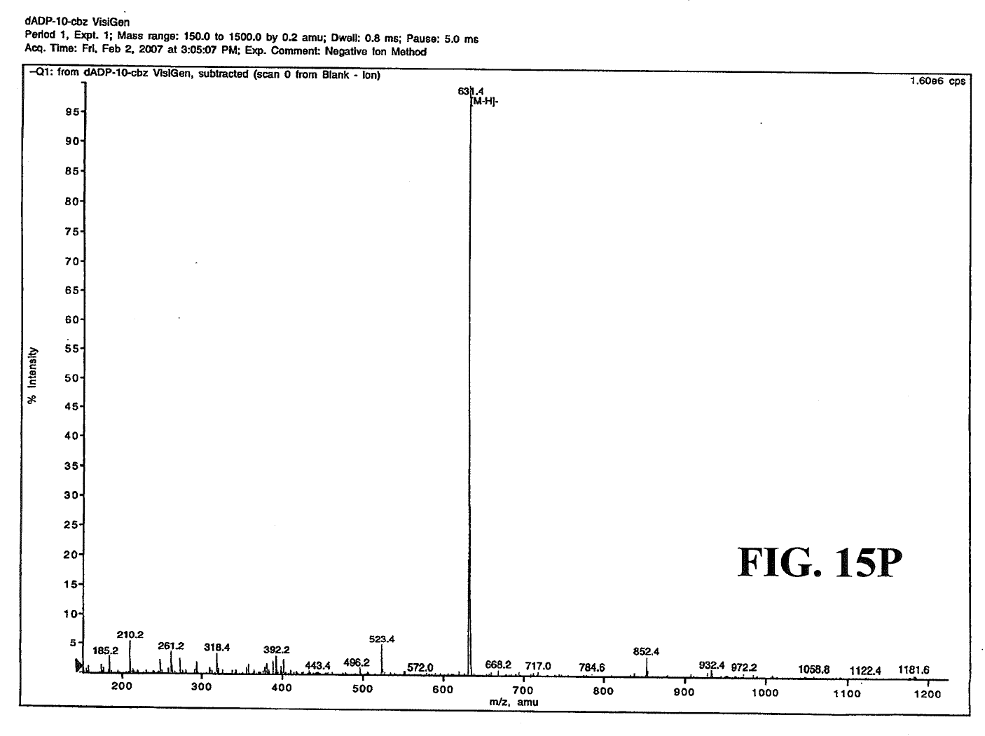

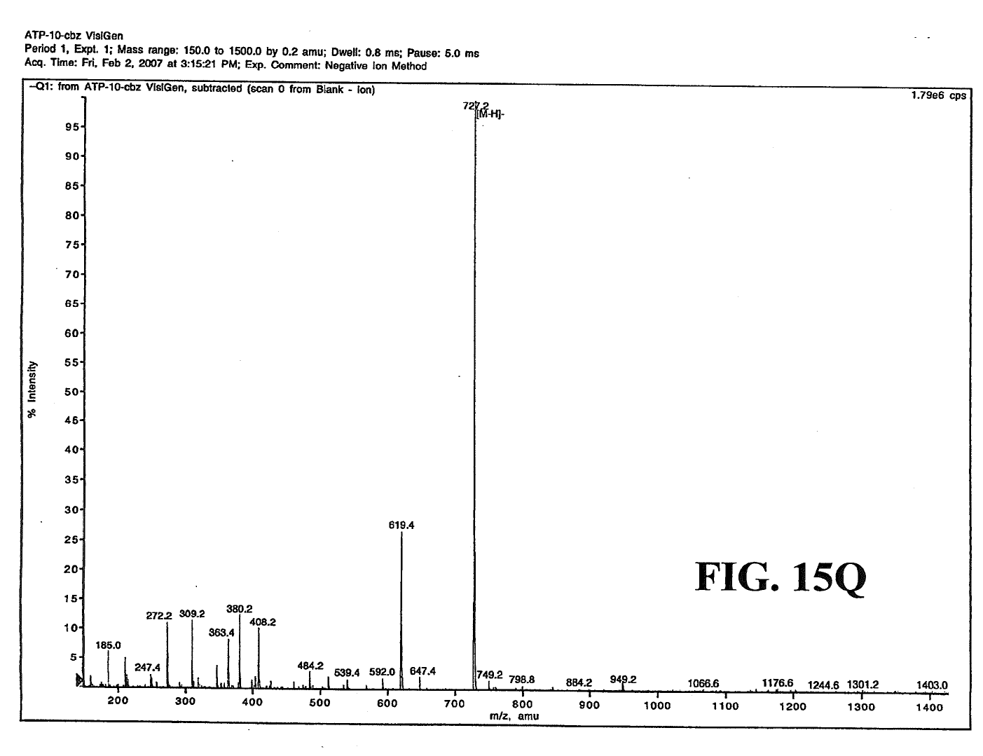

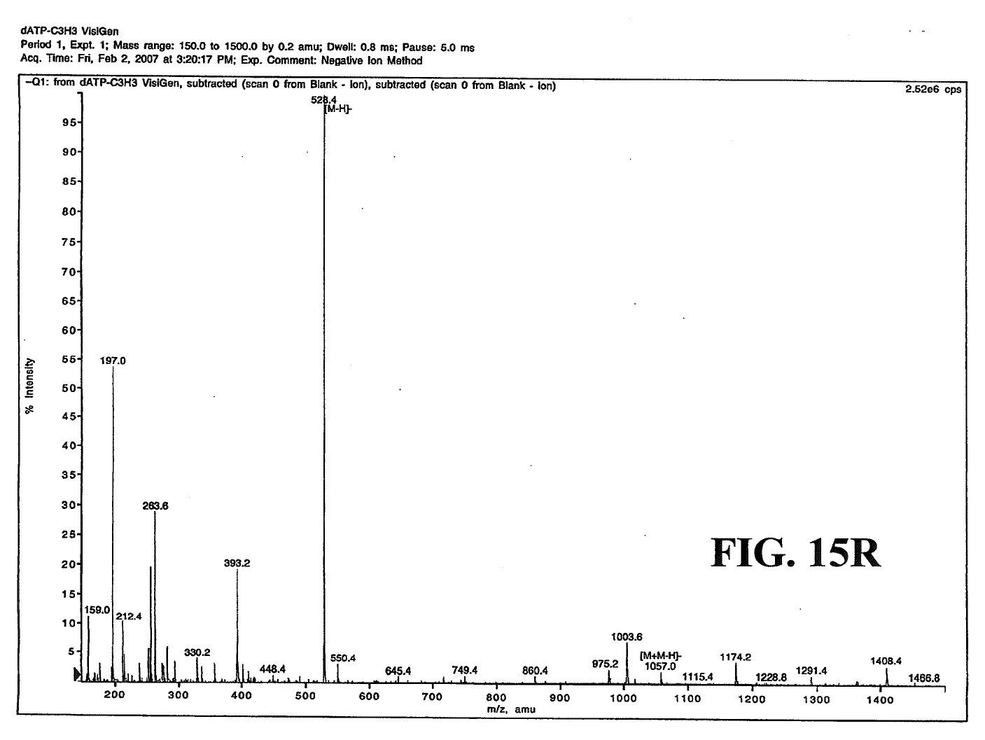

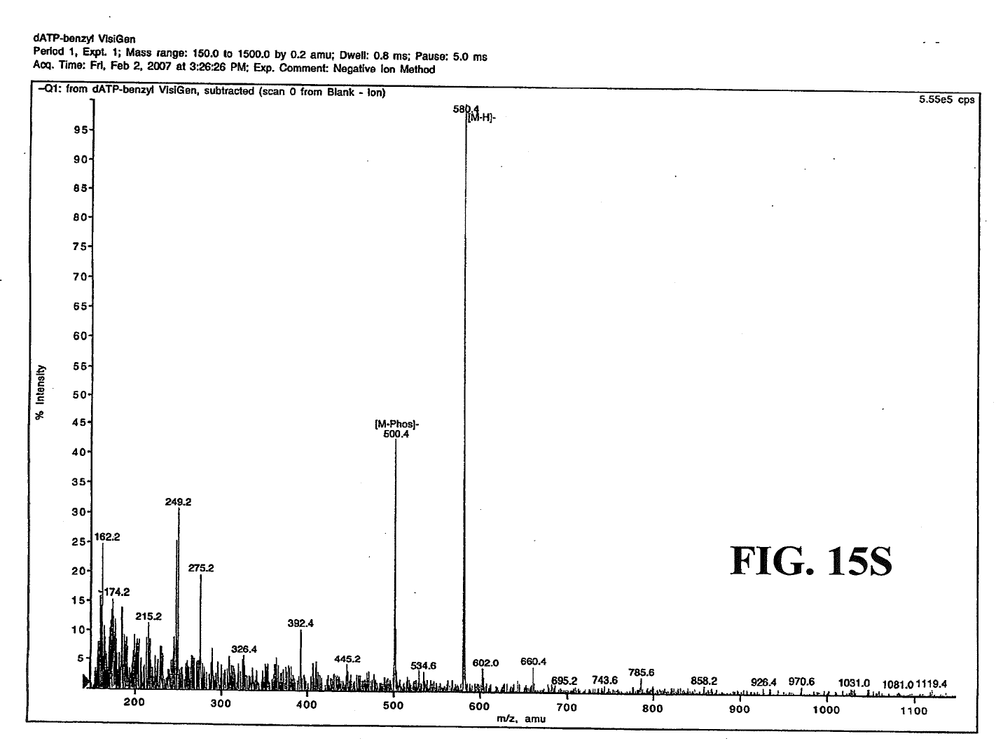

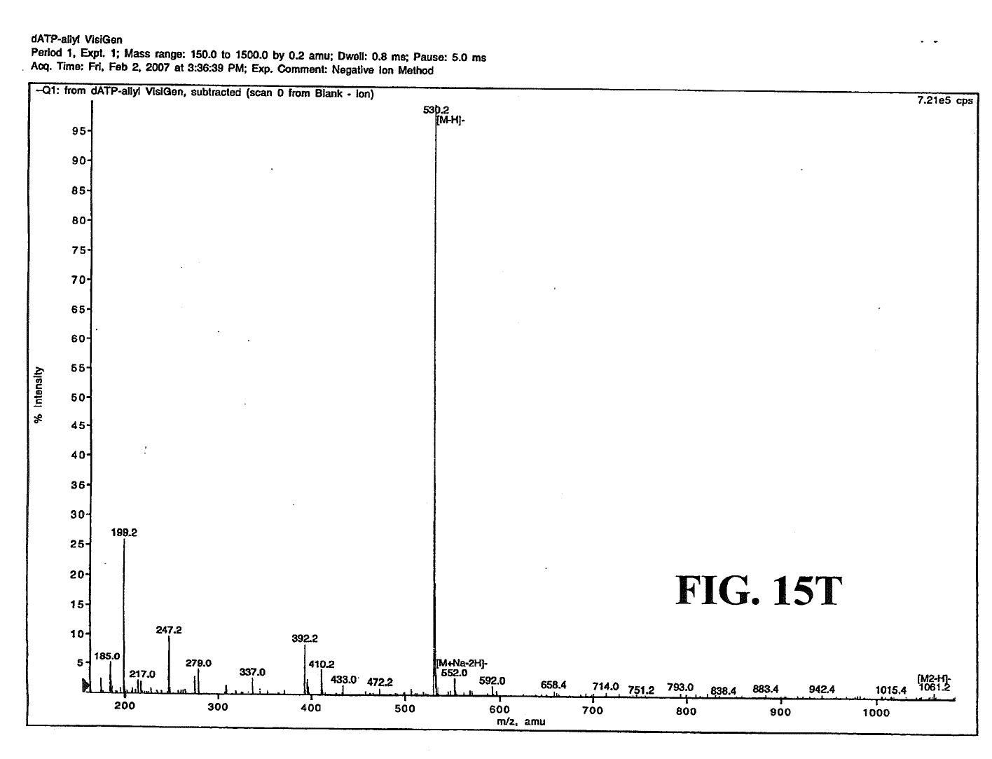

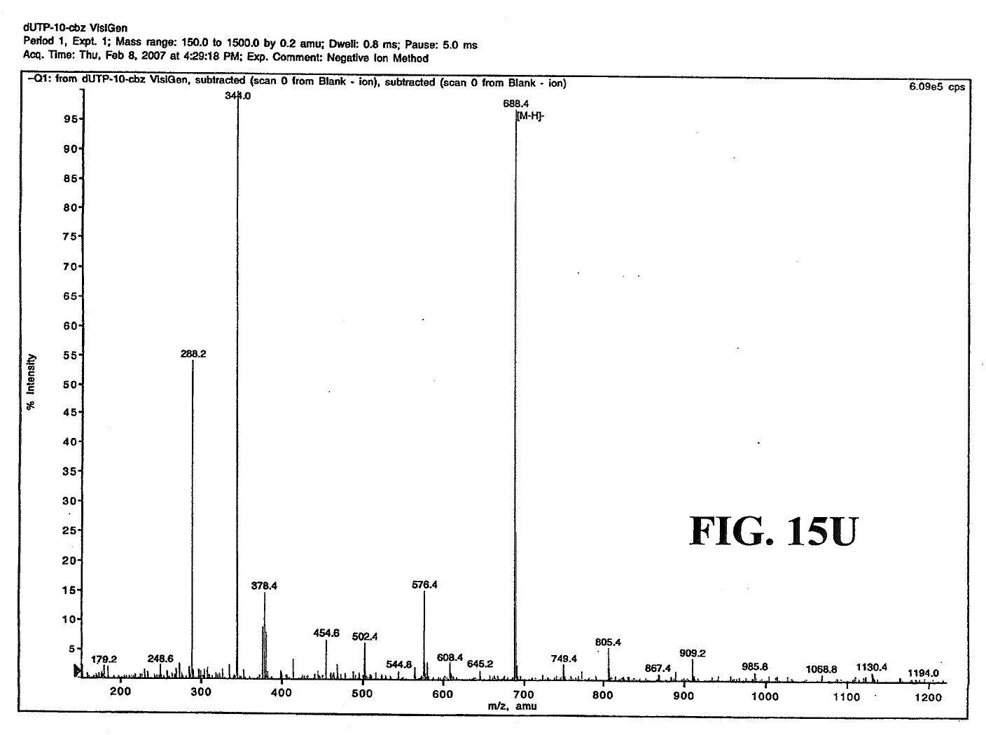

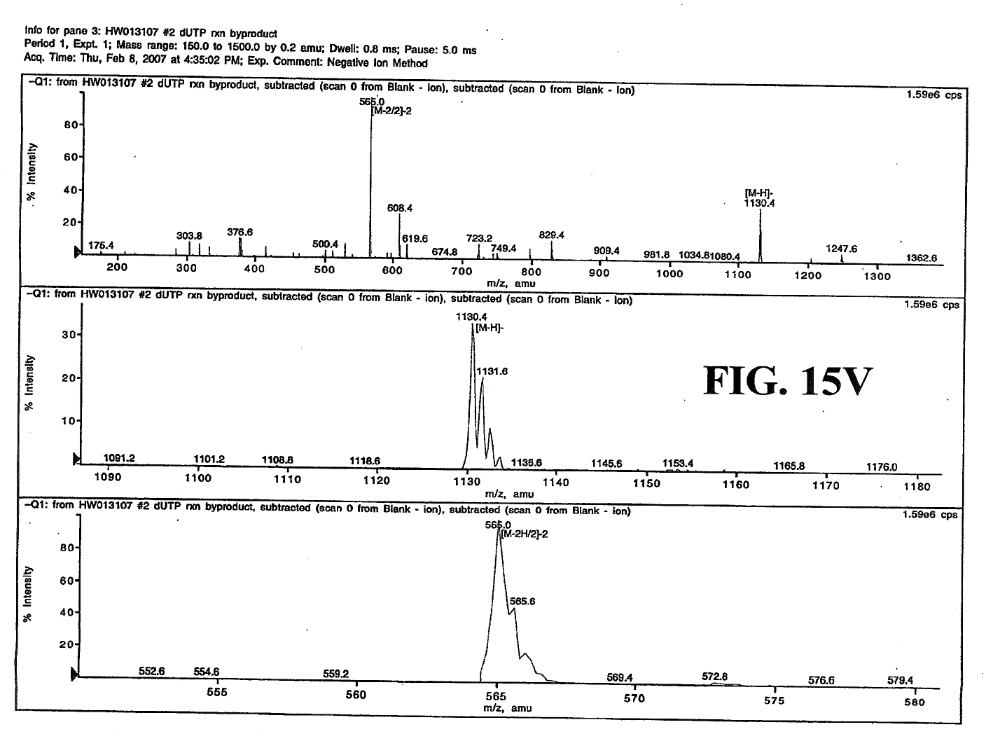

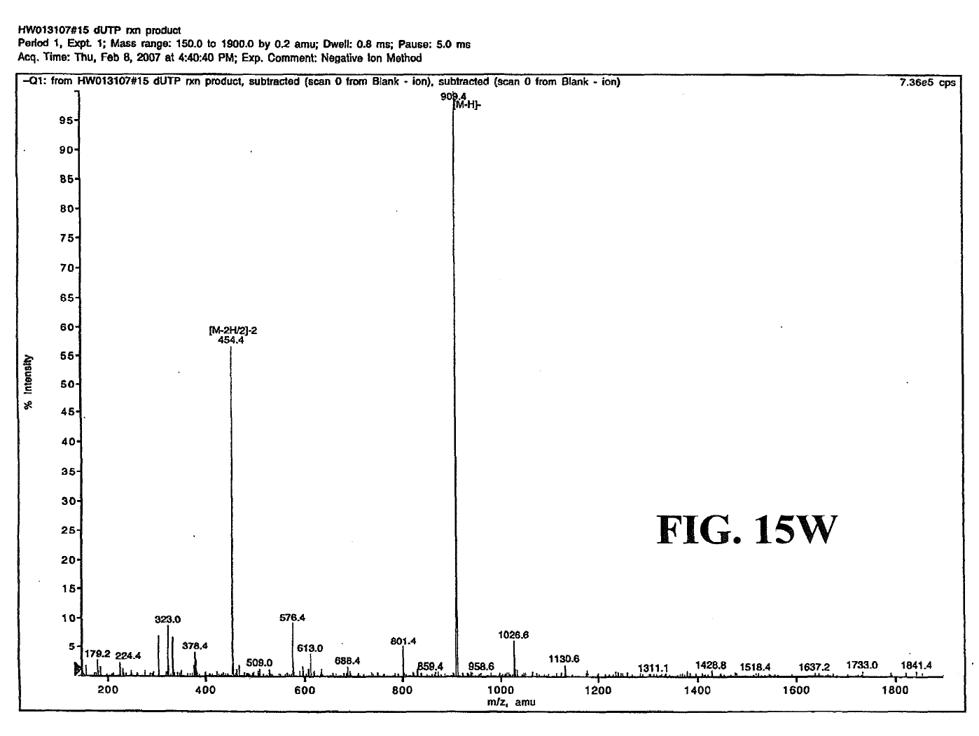

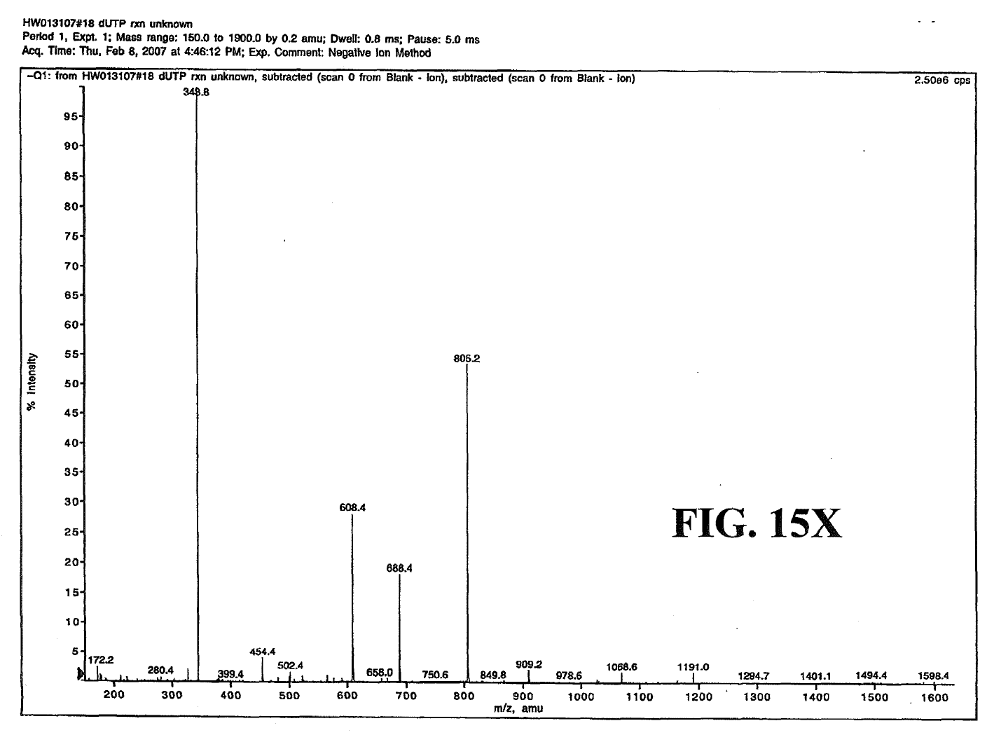

Under the designed experimental conditions alkylation on the gamma-phosphate dominates to give the desired product against possible alkylation on the base. The method has the following general characteristics: (1) the method is applicable to all five natural bases: A, T, C, G, and U; (2) the method is applicable to both deoxyribo- and ribo-nucleotides (Entry #8); (3) the method is applicable to other nucleotides (Entry #9, 10, 29, 31); (4) substrate concentration affects product yield (Entry #3, 21, 27, 28); (5) moisture or water lowers yields significantly (Entry #25 & 26); (6) nucleotide phosphate protonation state affects product distribution (Entry #7 & 31), a one-step method to doubly modify a nucleotide in both gamma- and base-positions; (7) solvent selection affects yields with polar aprotic solvents giving better results (Entry # 15, 18, 21, 23), surprisingly protic solvents also give products (Entry #17 & 24) while HMPA yields no product under experimental conditions (Aldrich 99% HMPA was treated with 2 batches of 4A MS over 48 hrs); (8) different leaving groups were presented: Ms, Ts, Br, etc.; and (9) different substrate structures were presented representing a wide variety of reactivities: PhCH₂—, CH₂═CHCH₂, CH≡CC—, PhCH₂CH₂—, Cbz-NH—CH₂CH₂OCH₂CH₂. FIG. 14 shows the structure of sixteen of the compounds set forth in Table 1. FIGS. 15A-X are mass spectra of the compounds of FIG. 14.

In conclusion, a facile, novel and general approach has been developed to generate NTP-γ-esters and other biological phosphate esters. As the need grows in biotechnology for these molecules, this approach will find many applications for its delivery in speed and simplicity.

The reactions were run by transferring mesylate with DMF into nucleotide tetrabutylammonium salt and stirring the resulting mixture at r.t. overnight. They were analyzed and the products were purified by HPLC (SAX or C18). All the products were characterized by mass spectrometry and gave the expected molecular weights (service by ABI). Every product was carefully assayed with phosphatases (CIAP and PDE1) coupled with silica TLC (two developing systems & phosphate staining). These experiments ruled out the possible base-modifications.

These experiments covered the following aspects in this chemistry development: (1) Bases: dATP, dTTP, dCTP, dGTP, dUTP; (2) Ribonucleotide: ATP; (3) Substrate leaving groups: mesylate, tosylate, bromide; (4) carbon types being attacked: saturated, allyl, benzyl, propynyl; and (5) other nucleotide: dADP.

It can be seen from the table that the yields are dependent on the nucleotide concentrations, substrate equivalents, and reaction times (18-42%). Leaving group reactivities and attacked carbon reactivities also have corresponding impacts.

The work will be focused on the other aspects of this chemistry including: (1) Solvent effects: CH3CN, NMP, MeOH, HMPA, etc.; (2) cation types: Et4N+, Mn+, Bu3NH+, Et3NH+, etc.; (3) nucleotide charges: dATP(Bu4N+)4, dATP(Bu4N)2, etc.; and (4) other phosphate species: DAMP.

New Compounds Prepared Using this Chemistry

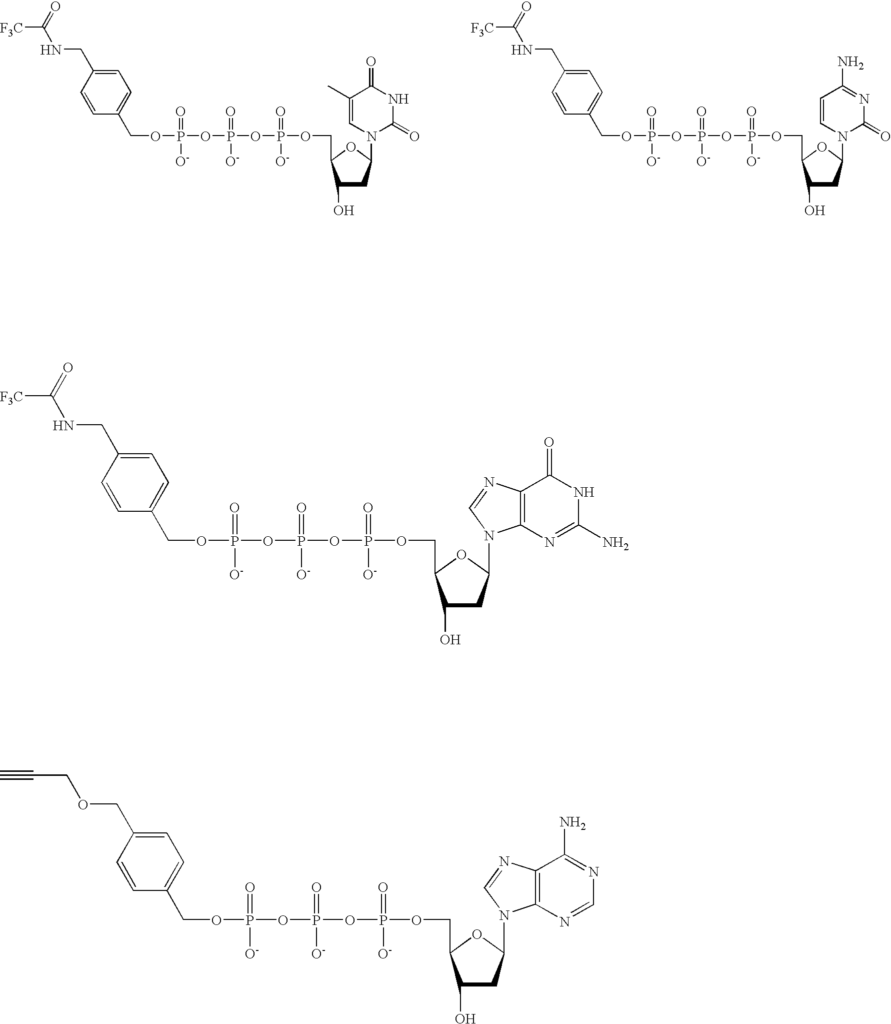

The nucleotides generated via this chemistry have wide applications in many aspects: click chemistry, fluorescent/quencher labeling, nucleotide structure/property modifications, star-molecule preparations, and other bioconjugations.

### Morpholidate Chemistry

In the 1960's phosphoromorpholidates were introduced by Moffatt and Khorana for the activation of nucleotide phosphates towards attack by a second phosphate (inorganic or other). See J. G. Moffatt and H. G. Khorana, JACS, 83:649 (1961) for further detail. They were also cursorily examined for attack by alcohols, but the reactions found to require a large excess of alcohol and proceed quite slowly, if at all. In general, the morpholidate chemistry is very slow (reaction times of a week is not uncommon). In 1997, Chi-Huey Wong showed that tetrazole is an effective catalyst for the reaction between phosphoromorpholidates and sugar-phosphates. See V. Wittmann and C—H. Wong, J O C, 62:2144 (1997) for further details. Building on this, we have used tetrazole as a catalyst for the reaction between phosphoromorpholidates and alcohols. This method for modifying the gamma-phosphate of a dNTP has the advantage of using the free alcohol of the linker, rather than an activated linker (phosphate, leaving group, etc.), and uses an easily made, storage-stable reactive dNTP intermediate. Phosphoromorpholidates of some NMPs are even commercially available, which demonstrates their feasibility as a product to make and store in bulk.

### General Morpholidate Synthetic Method

### Example 2

This example illustrates a general morpholidate method for preparing phosphate modified biomolecules, with deoxycytidine as the biomolecule and CbzN(H)CH₂CH₂OH as the modifying agent.

10 μmol of the triethylammonium salt of deoxycytidine-5′-phosphoromorpholidate, which was synthesized according to the method described in J. G. Moffatt and H. G. Khorana, JACS, 83:649 (1961), passed over a bed of DOWEX 50W8-200 ion exchange resin which had been equilibrated with 1M Et₃N·AcOH, and lyophilized, was coevaporated with methanol, ethanol, and three times with pyridine. 50 eq of alcohol 1 was then added, and the mixture coevaporated with pyridine once, then dried under vacuum for 3 hours. Pyridine (100 μL) and tetrazole (2 eq, 0.45M in acetonitrile) were then added, and the reaction stirred at room temperature for three days. After this, the reaction is quenched with water, and extracted three times with ether to remove excess alcohol, and dried to a residue. This was taken up in water and purified directly by reverse-phase HPLC.

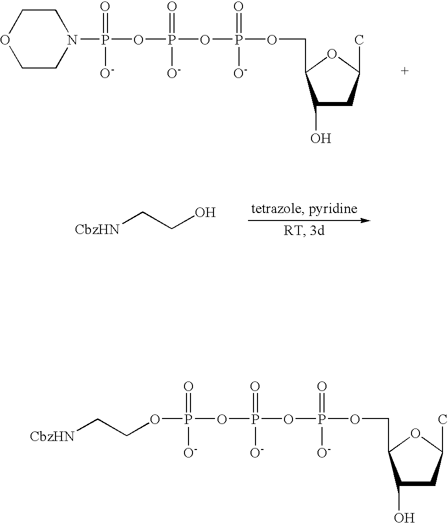

This method has been used to make compounds 2-4 shown below. The products have been characterized by enzymatic activity (all) and mass spectral analysis (compounds 3 and 4). These compounds demonstrate that this method works for both aliphatic and benzylic alcohols.

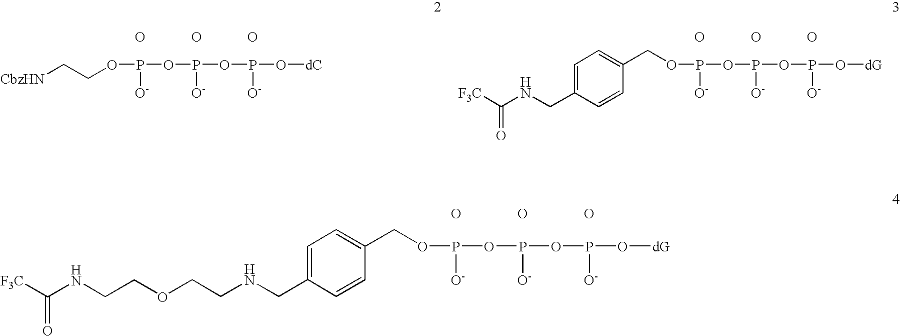

An evaluation of the optimum equivalents of tetrazole and linker shows that 1 eq of tetrazole is preferred, and that 3 eq of linker are sufficient for generating 21% of desired product. Additional linker does not increase product yield, and additional catalyst leads to decomposition of the starting material.

The following additional gamma-dNTPs have been made using the morpholidate chemistry:

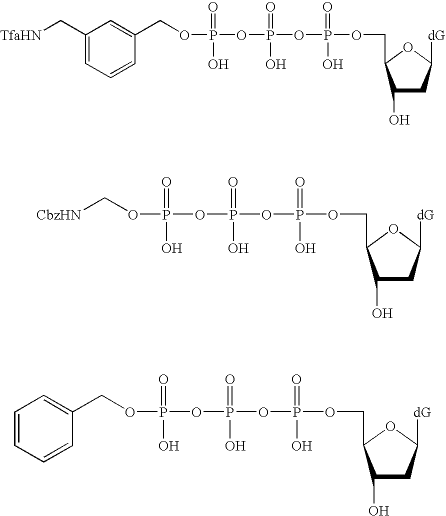

dNTP as Nucleophile Chemistry

The following gamma-dNTP has been made using the dNTP as nucleophile chemistry with a tosylated linker:

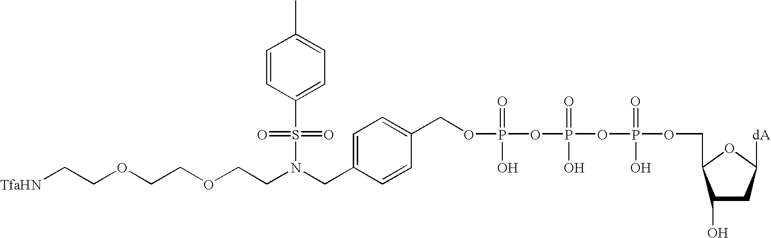

Dual-Labeled dNTP Synthesis

The dual labeled dNTP was synthesized in an attempt to monitor energy transfer from a stable donor (quantum dots) to Cy3 and then on to Cy5. The donor would be stable enough for real-time detection and the FRET between Cy3 and Cy5 should be very efficient. The candidate chosen to be synthesized was dA-2-Cy3-2-Cy5.

In the first step, p-xylylenediamine (0.3 mg; 2.4 μmol) was dissolved in MeOH/TEA anhydrous (3/1) and the solvent was evaporated first on the rotavap, than under high vacuum for a couple of hours. Once dried, p-xylylenediamine (Linker 2) was dissolved in DMF anhydrous (100 μL), and a Cy5-NHS (0.4 μmol) solution in DMF anhydrous was added to the flask in presence of TEA (30 μL). The reaction mixture was shaken overnight in the dark. The reaction was conducted in a large excess of Linker 2 (α,α′-diamino-p-xylene or p-H₂NCH₂PhCH₂NH₂) to insure that the major product would be the mono-substituted Linker 2. The reaction mixture was purified by HPLC using the C18 column and the desired product was identified after purification by TLC. The reaction yield was 17% (68 nmoles).

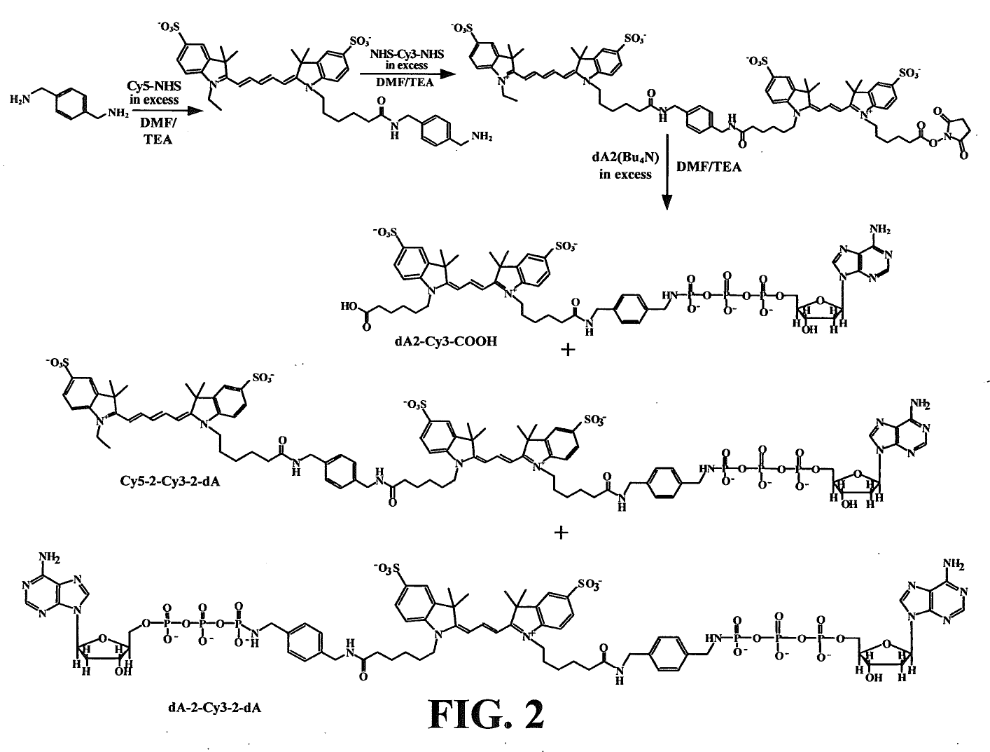

In a second step, Linker 2-Cy5 (20 nmoles) was coupled to Cy3-bis NHS (in excess, 3 eq.) in DMF/TEA anhydrous (4/1). The reaction mixture was shaken overnight in the dark. The reaction was followed by TLC until the Linker 2-Cy5 spot disappeared. A new purple spot (Cy5 blue+Cy3 pink) appeared which seemed to indicate that the desired intermediate, (NHS)-Cy3-Linker 2-Cy5, was formed. The reaction mixture was not purified to avoid hydrolyzing the NHS groups. Note that in the reaction mixture, there is an excess of Cy3-bis NHS, which is available to make other interesting molecules as indicated in the synthesis scheme shown in FIG. 2. Linker 2 is sometimes abbreviated L2.

In a third step, dA2(Bu₄N⁺)₃ was dissolved in DMF anhydrous and added in large excess (0.27 umol; >10 eq.) to the reaction mixture. After stirring overnight in the dark, a new spot appeared on the TLC, a smearing pink spot, which would correspond to a labeled dNTP. The reaction mixture was purified by HPLC using a SAX column. Several fractions were collected with absorptions at either 260 nm only, 260 nm+548 nm, 548 nm+647 nm, or 260 nm+548 nm+647 nm. All the fractions were resuspended in 10 mM HEPES and a PDE enzyme test was performed to identify the different fractions using a silica TLC plate for detection. As expected, the desired product was one of the later peaks on the HPLC spectra, with absorption at both 548 nm and 647 nm. The yield of the reaction was low (7%, 1.4 nmoles).

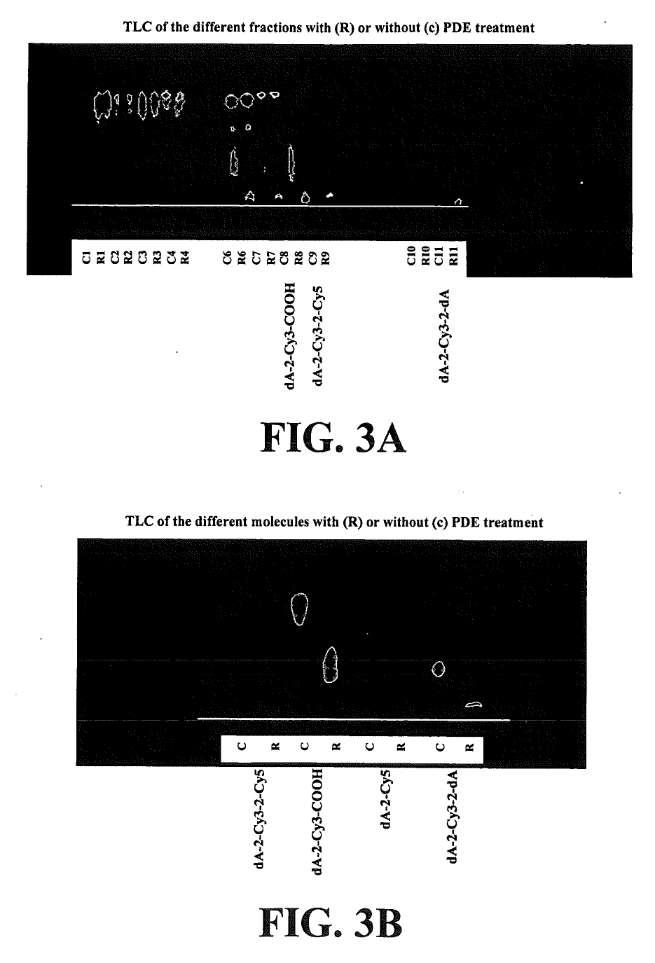

Note that other molecules were isolated and identified as products of this reaction: dA2-Cy3-COOH (5.3 nmoles), and dA-2-Cy3-2-dA (star molecule, 2.8 nmoles) which is the last fraction to come out on the HPLC and migrate much lower than dA-2-Cy3 on PEI cellulose TLC plates, as shown in FIGS. 3A&B. The star molecule was determined by the inventors to be a potential way to double the amount of dNTP in the reaction mix without increasing fluorescence concentration, thereby lower background fluorescence.

After the products were positively identified, steps 2 and 3 were repeated with the remaining Linker2-Cy5 to generate more products. The yields were similar as the ones obtained previously.

### Synthesis Scheme of the Star Molecule

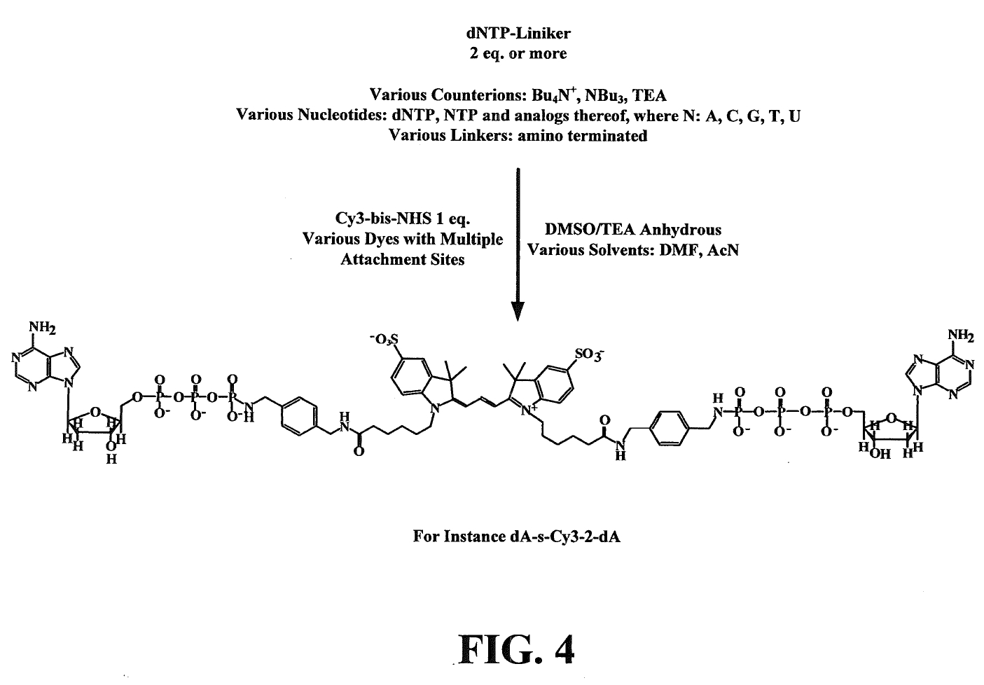

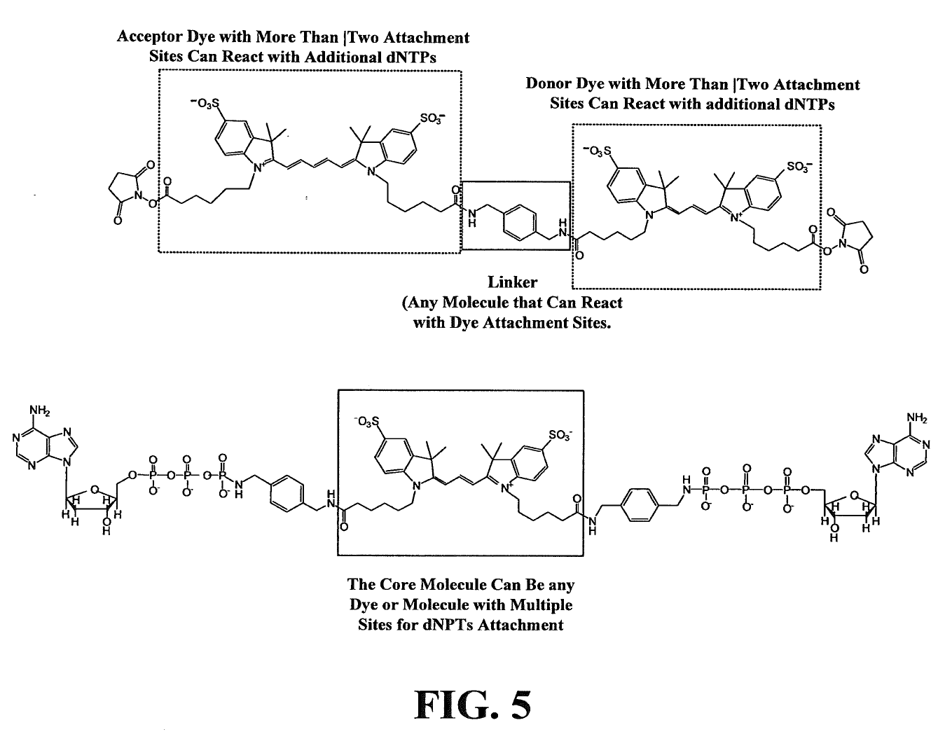

A gamma-modified dNTP with amino alkyl counter ions is dissolved in an anhydrous polar solvent such as DMSO, DMF, or AcN, in a slight excess compared the bis-reactive dye, in presence of TEA. The reaction mixture is stirred overnight, in the dark, at room temperature. The reaction is followed by TLC. Upon completion, the reaction mixture is purified by HPLC. The synthetic scheme is shown graphically in FIG. 4. In FIG. 5, several examples of core structures that can be used to prepare star molecules are shown. Unexpectedly, higher reaction yields were obtained when the reactions were performed in a solvent mixture of DMF and sodium bicarbonate buffer (130 mM final concentration), instead of anhydrous organic solvents.

### Molecules Analysis

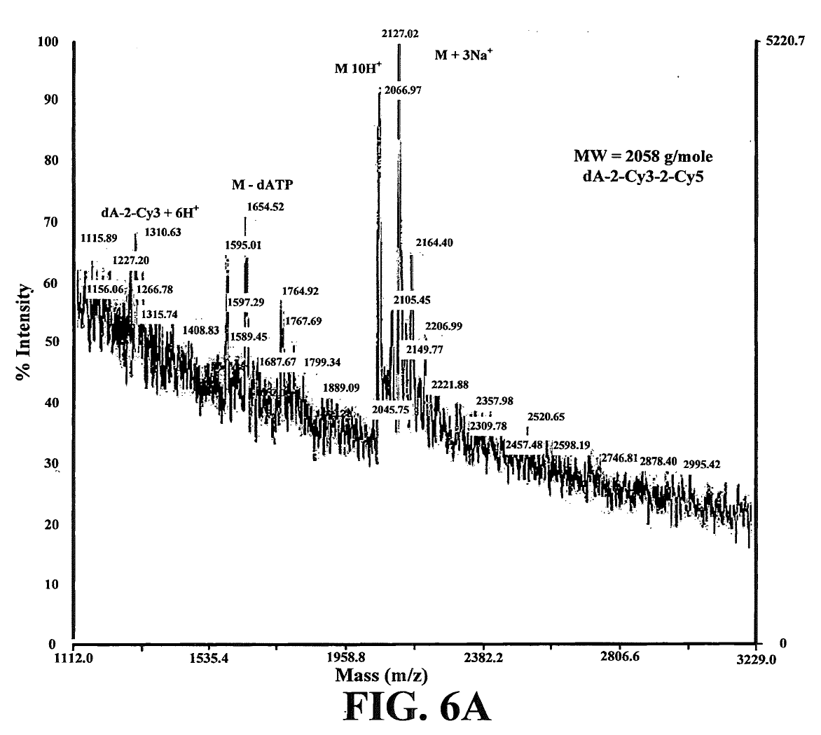

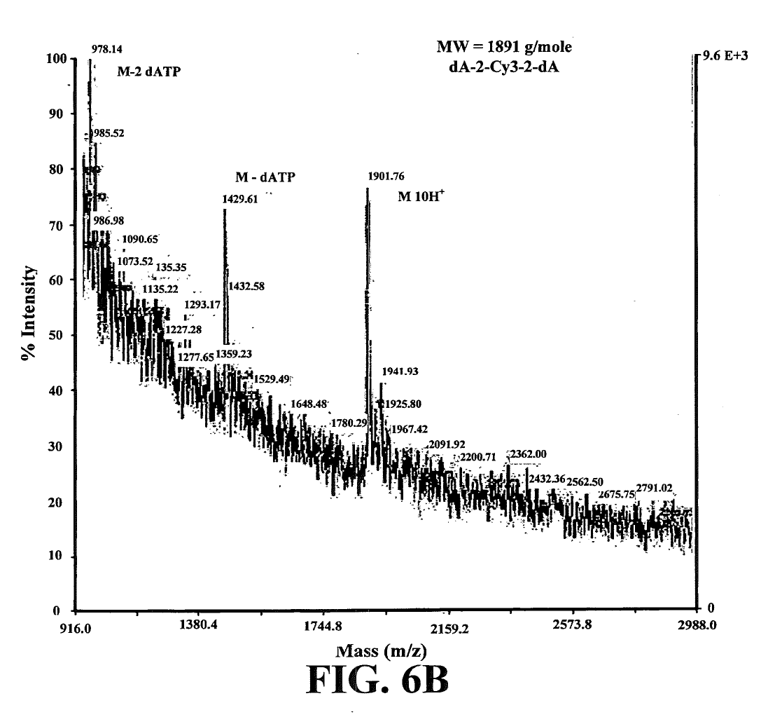

The dual-labeled dNTP, dA-L2-Cy3-L2-Cy5, (MW: 2058 g/mol) and the star molecule (MW: 1891 g/mol) were both sent for mass analysis (MALDI), which confirmed that these molecules were the identified products as shown in FIGS. 6A&B, respectively. Cy3 and Cy5 are commercially available fluorophores.

The molecules UV absorption spectra were obtained. It was noted that for the dual-labeled molecule that absorption at 548 nm was 2-3 times higher than the 647 nm absorption. This was not expected since the extinction coefficient of Cy5 is 2.5 times larger than the extinction coefficient of Cy3. On the TLC plate, one can see that there is some free Cy3 in this fraction, which may explain a higher Cy3 absorption, but not to the extent observed. However, no side products were detected on the MALDI mass spectra.

### Example 3

This example illustrates an extension reaction using the dual labeled nucleotide dA-L2-Cy3-L2-Cy5 and the dual nucleotide star molecule, dA-L2-Cy3-L2-dA.

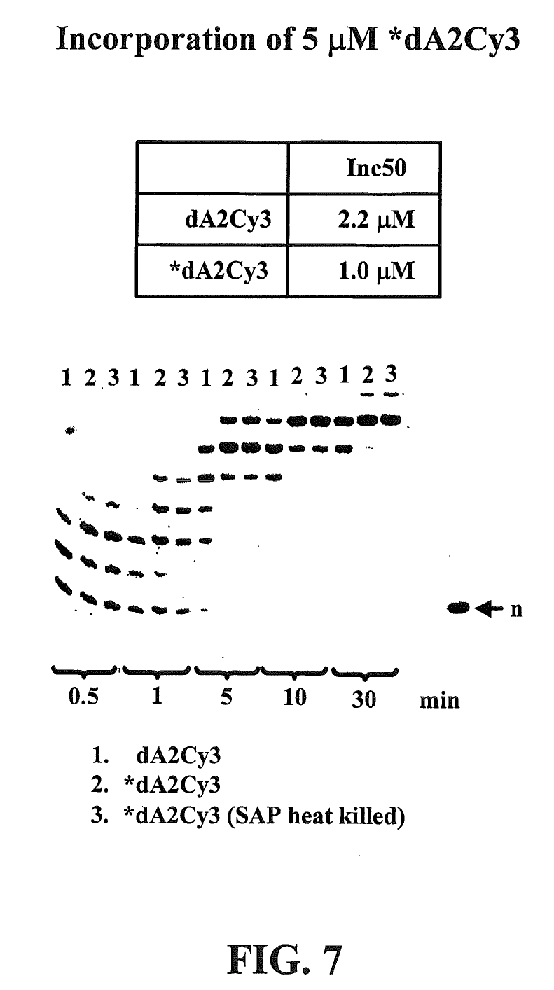

The Inc50 concentrations were from 1.25 μM to 0.002 μM over 10 reactions. The *dA2Cy3 [/2] indicates that the stock concentration was based on dA and not Cy3. In the 7 base extension the stock concentrations of *dA2Cy3 was based on Cy3 and were at 5 μM. The 7 base extension was also repeated at more limiting dNTP concentration, however the above assay indicates there is more DATP present in the *dA2Cy3 preparation and that both dATPs are utilized. We are not sure if both dATPs are utilized at the same efficiency by Klenow. The Inc50 for the *dA2Cy3 was repeated in triplicate to confirm the Inc50 value. The results are shown in FIG. 7.

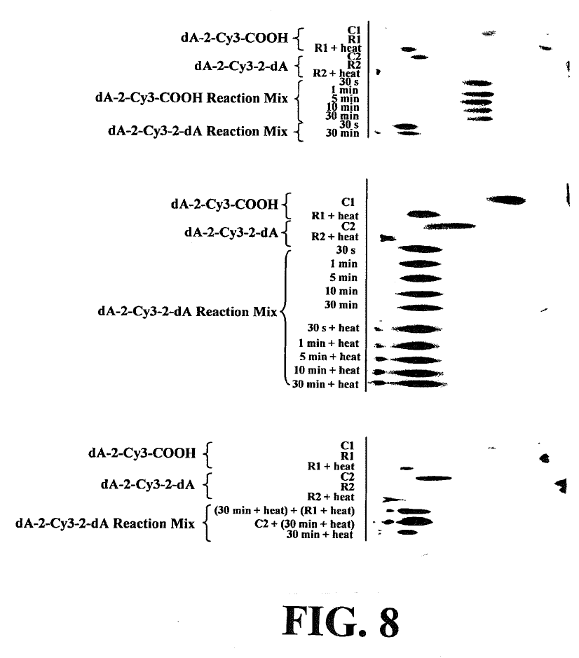

Using the solution obtained for the time points, but quenched with EDTA, the release products were followed by TLC. The TLC was run on PEI-cellulose glass plates, using EtOH (35%) in 1M TEAB as eluant. The results are presented as shown in FIG. 8. The nucleotides dA-2-Cy3-COOH and dA-2-Cy3-2-dA were treated with PDE1 and used as controls. Results show that when SAP remains active the PPi-product gets cleaved and free dye is released. Therefore the PPi-product release cannot be followed under these conditions. When SAP is heat killed, the PPi-product remains intact. We can see that the amount of PPi-2-Cy3-2-PPi released increases with longer reaction time. This indicates that with time, both sides of the star molecules are incorporated. Since the nucleotides are in large excess compared to the duplexes, they remain in the reaction mix, even after incorporation has occurred, as seen on the TLC. Note that the star molecules migrate differently depending on the buffer it is diluted in. In PDE buffer (C2), the star molecule migrates higher compared to the reaction buffer (time points) because of the presence of magnesium ions; but, when co-spotted they migrate together to the lower height. This explains why the PPi-2-Cy3-2-dA intermediate spot cannot be distinguished from the star molecule spot, they both run together on the TLC. The TLC plates are shown in FIG. 8. In FIG. 8, C represents nucleotide in PDE buffer, R represents nucleotide in PDE buffer with PDE1 enzyme and Heat represents SAP was heat killed before the extension reaction was run.

### Example 4

This example illustrates an extension reaction using dual nucleotide star molecule of this invention, dA-L2-Cy3-L2-dA, using Klenow and a variant thereof.

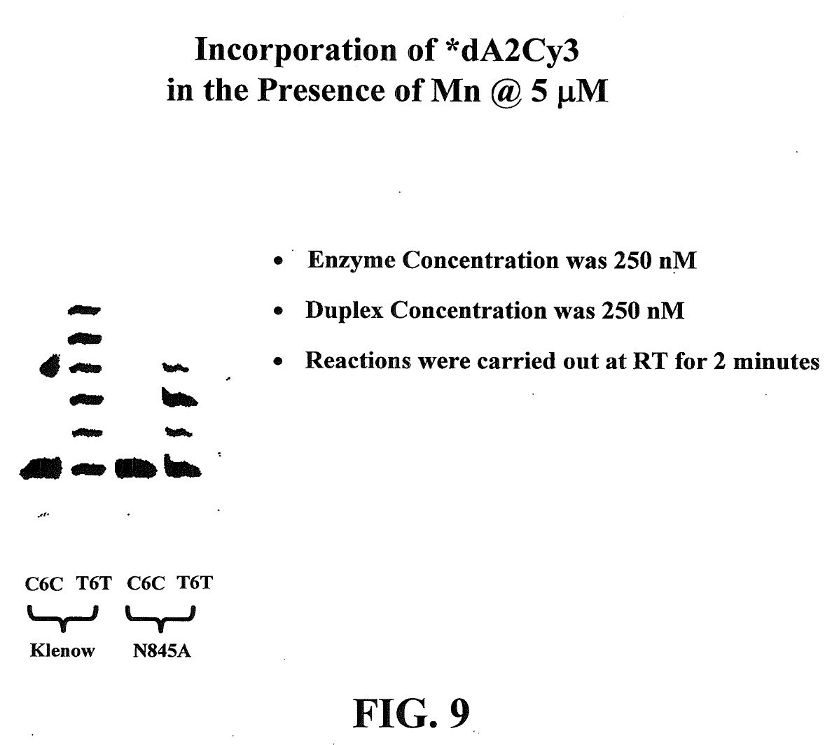

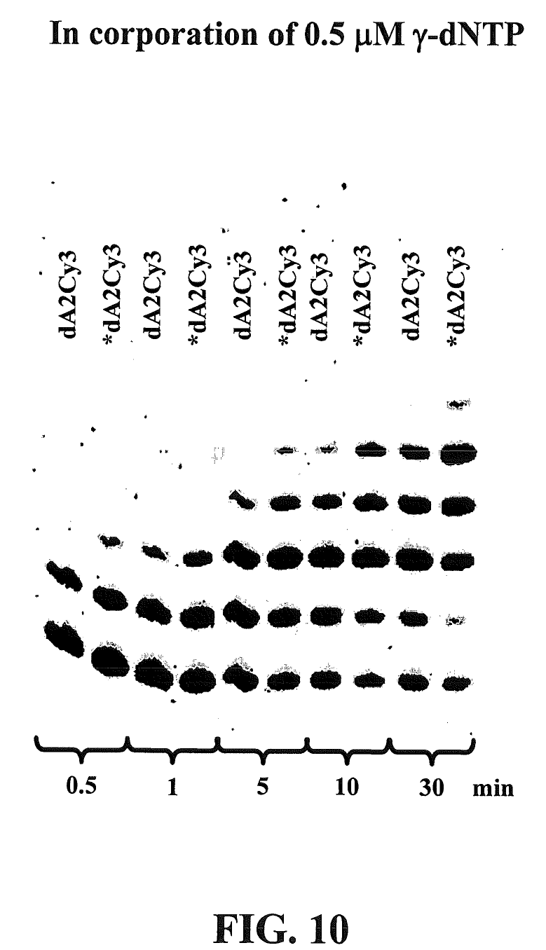

Klenow Concentrations of Inc50 175 nM and Duplex Concentrations of Inc50 10 nM in a 7 base extension and combos 250 nM. Inc50s are quenched at 1 minute, and on the last slide everything was quenched at 5 min. The others the times are noted because there were multiple time points. Components: 50 mM Tris (pH 7.0), 1 mM MnCl₂, 100 μM DTT (Inc50s included 1 mM Spermine). The dNTPs used, dA-L2-Cy3-L2-dA (*dA2Cy3) and gamma-labeled dA-L2-Cy3, and their concentrations are indicated. All reactions were performed at room temperature. The results of these test are shown in FIG. 9 and FIG. 10.

### Example 5

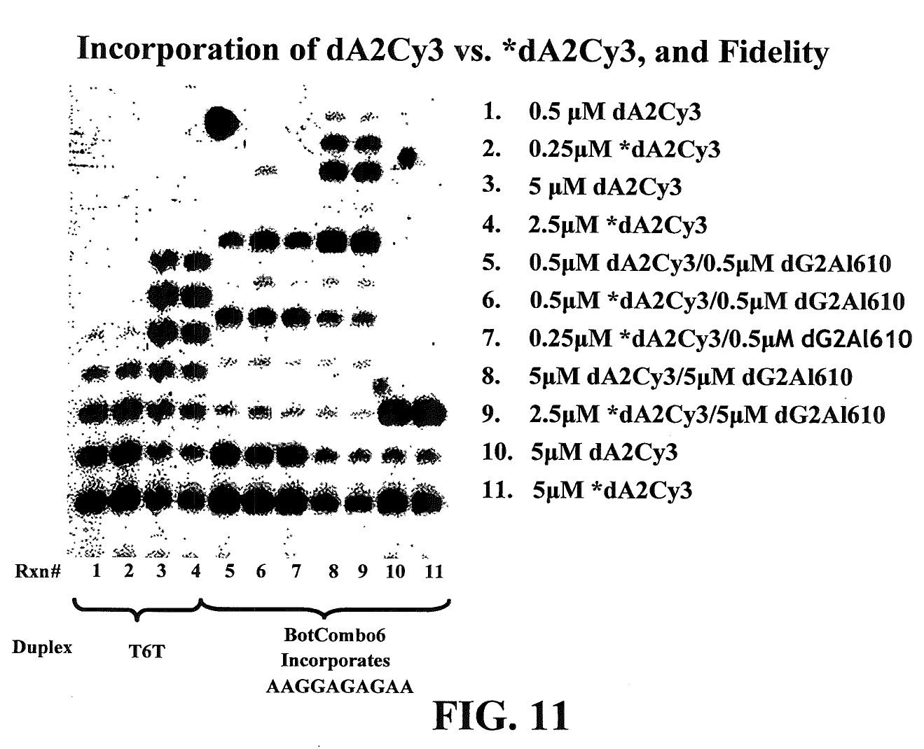

This example illustrates an extension reaction using the gamma-labeled nucleotide dA-L2-Cy3 and the dual nucleotide star molecule, dA-L2-Cy3-L2-dA (*dA2Cy3) either alone or in combination with gamma-labeled dG-L2-Al610. This example illustrates representative extension reactions that demonstrate that the nucleotides attached to the star molecule and the gamma-labeled nucleotides are accurately incorporated. The results are shown in FIG. 11.

### Example 6

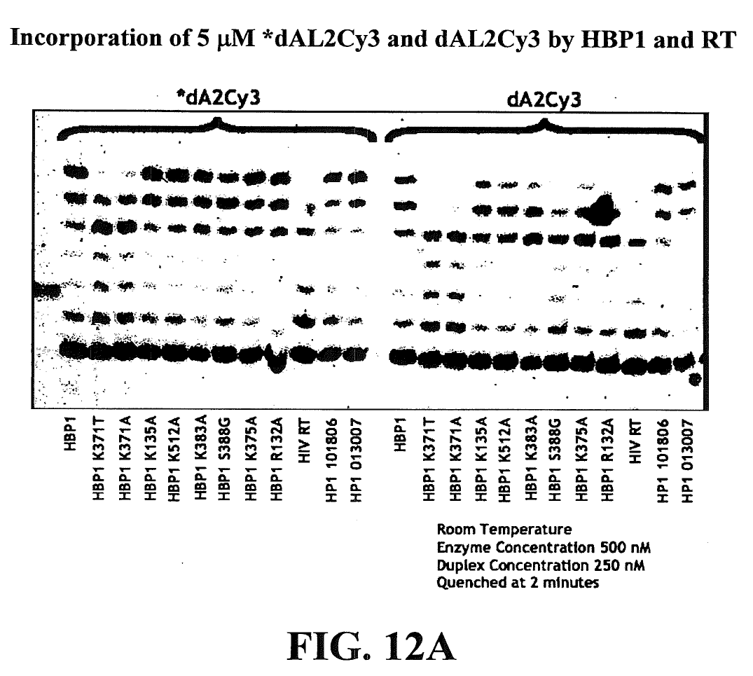

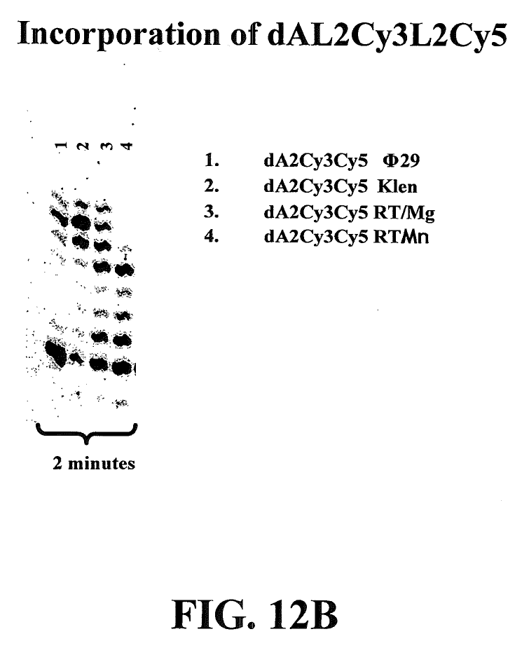

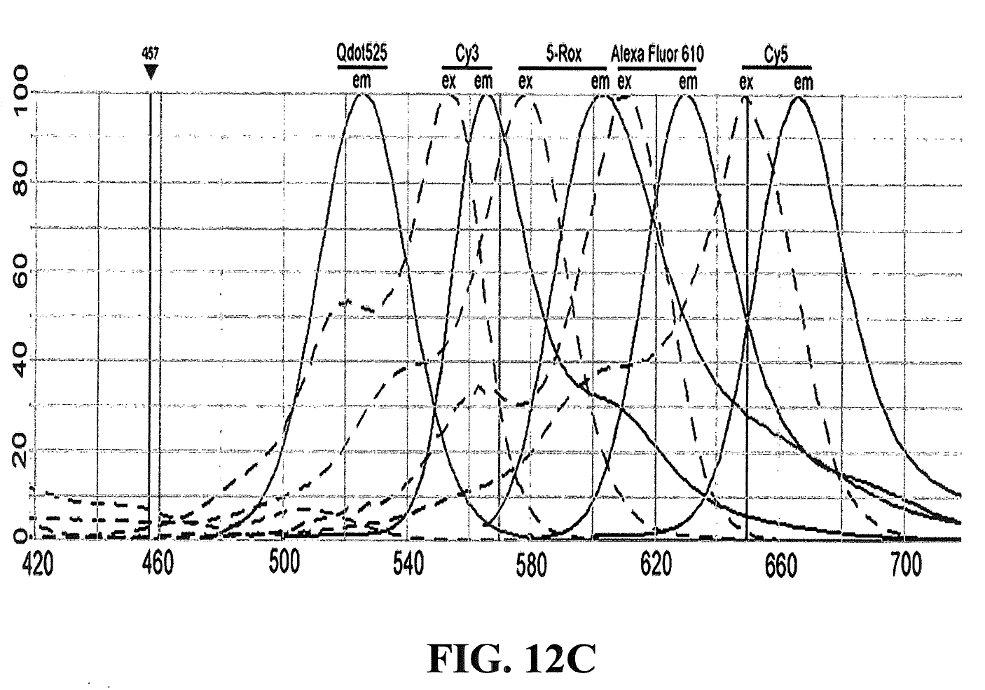

This example illustrates extension reactions using the gamma-labeled nucleotide dA-L2-Cy3 (dA2Cy3) and the dual nucleotide star molecule, dA-L2-Cy3-L2-dA (*dA2Cy3) using phi29 variants and HIV-RT. Interestingly, phi29 variants efficiently use the star molecule to different extents, whereas HIV-RT appears to use the gamma-labeled dA2Cy3 more efficiently. These results are shown in FIG. 12A. In FIG. 12B, primer extension reaction are shown for dA-L2-Cy3-L2-Cy5 (dA2Cy3Cy5).

The present invention also relates to a FRET strategy utilizing nucleotides having dual tags or labels at their gamma phosphate such as the Cy3 and Cy5 nucleotide discussed and synthesized above. These dual labeled nucleotides are designed to be used in “triple-FRET” strategy using a Quantum dot as a persistent donor. The inventors believe that the triple FRET strategy will have the following advantages: (1) further reduce background fluorescence because a lower wavelength laser is used to drive the energy transfer (less direct excitation of acceptors); (2) produce stronger acceptor FRET because Cy3 is a better FRET pair than 488 with all of our acceptors; (3) the use of a Qdot is expected to provide a stronger signal by providing more energy into Cy3 (acceptor #1); and (4) lower laser power can be used to excite the Qdot, further reducing background fluorescence.

All of the above advantages will give rise to primer extension reaction having improved signal to noise ratio and, thus, detectability.

The dual label configuration may also allow us to obtain relatively similar detectability between the different acceptors due to the ability to control spacing between Cy3 (acceptor #1) and the identifier acceptor dye—both of which are attached to the gamma phosphate.

The quantum dot used as the donor will be near or associated with an immobilized or confined replication complex (polymerase/primer/template).

The commercially available bis-Cy3 allowed the necessary linkers to be added on the same side of the molecule, creating a more ‘U’ shaped molecule that, unfortunately is not well incorporated by Klenow (other enzymes are being tested). Minimally, this dual-dNTP will serve to characterize triple-FRET and its potential benefits.

As shown in FIG. 12C, a commercially available quantum dot Qdot 525 effectively excites Cy3, which effectively excites Cy5, via FRET allowing the quantum dot to induce FRET in the Cy3Cy5 dual labeled dNTP as it moves into the local vicinity of a replication complex associate with or attached to quantum dot.

### Example 7

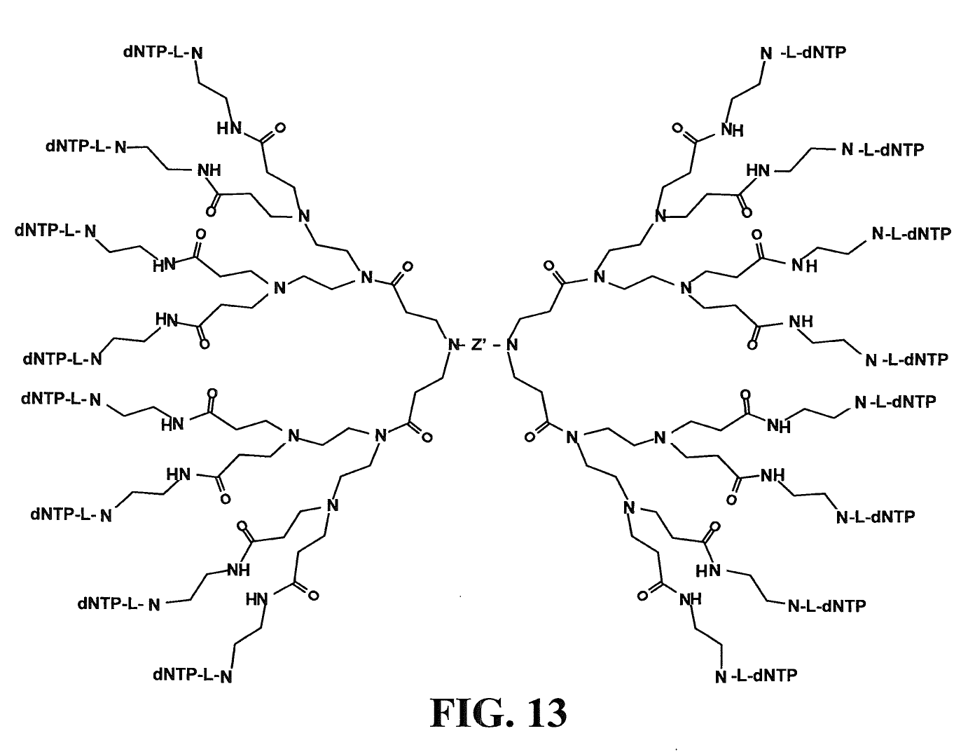

This example illustrates a dendrimer that is modified to produce a star molecule with 16 nucleotides attached via linkers. The results are shown in FIG. 13.

### Triple Fret Test Compound

A non-nucleotide compound was synthesized to test the FRET efficiency of linked fluorophores (Scheme B). The inventors first tried to synthesize the compound in a one-pot reaction by staggering the addition of the two fluorophores to linker 2. Unfortunately, no product was recovered. The reaction was repeated, but instead, purified linker 2-Cy3b (mono-functional cy3) intermediate was added before adding ROX-NHS. The desired product was isolated, which underwent spectroscopic characterization. Cy3b is a commercial mono-functional dye from Amersham Biosciences a GE Healthcare company.

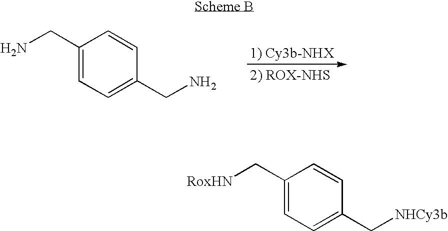

### New Compounds

Referring now to FIG. 13, a possible dendrimer structure including L-dNTP terminated arms is shown. Of course, one of ordinary skill in the art should recognize that this structure is possible using the synthetic approaches set forth above. At the center or core of the dendrimer is a Z group, a group having a detectable property such as a fluorescent dye or a donor-acceptor pair. If the core of the dendrimer is a fluorophore that interacts with a fluorophore on the polymerase, proper spacing between the donor and acceptor must be maintained to obtain efficient (detectable) FRET.

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

We claim:

1. A method for preparing a modified nucleotide comprising the step of:
contacting a biomolecule having a phosphate group and a modifying agent having a leaving group capable of being displaced by the phosphate group under displacement reaction conditions to form a phosphate modified biomolecule.

2. The method of claim 1, wherein the nucleotides, phosphorylated polypeptides, phosphorylated proteins, phosphorylated sugars or sacchrides, phosphorylated carbohydrates, phosphorylated enzymes, phosphorylated membranes, phosphorylated cells, phosphorylated tissues, phospholipids or any other bio-material or organized structure bearing at least one phosphate group or mixtures or combinations thereof.

3. The method of claim 1, wherein the modifying agent comprises:
a molecule having one attachment site or a plurality of attachment sites, each site bearing a leaving group.

4. The method of claim 3, wherein the molecule comprises a molecular core and the site or sites extend out from the core.

5. The method of claim 3, wherein:
the biomolecule comprises a nucleotide comprising a nucleoside or nucleoside analog having at least one phosphate group attached at its 5′ hydroxy group, and
the core comprises (a) one quencher or a plurality of quenchers, or (b) an acceptor fluorophore or a plurality of acceptor fluorophore, or (c) an acceptor fluorophore or a plurality of acceptor fluorophore and a donor fluorophore or a plurality of donor fluorophore for the acceptor or acceptors.

6. The method of claim 5, wherein the core is selected from the group consisting of bi-functional molecules, polyfunctional molecules, polyfunctional boron-nitride nanostructures, polyfunctional carbon nanostructures, polyfunctional dendrimers, polyfunctional oligomers, polyfunctional polymers, polyfunctional metal oxide nanostructures, polyfunctional quantum dots, polyfunctional metal clusters, polyfunctional nanoshells, polyfunctional liposomes, or any other structure that can support attachment of a plurality of nucleotides through their terminal phosphate or mixtures of combinations thereof.

7. The method of claim 5, wherein the quenchers are the same or different, the acceptor fluorophores are the same or different or the acceptor fluorophores are the same or different and the donor fluorophores for the acceptors are the same of different.

8. A method for preparing a modified nucleotide comprising the steps of:
contacting a biomolecule having a phosphate group and a linker having a reactive site protected by a protecting group and a leaving group capable of being displaced by the phosphate group under displacement reaction conditions to form a phosphate protected linker modified biomolecule, and
de-protecting the terminal phosphate protected linker modified biomolecule to form a terminal phosphate reactive linker modified biomolecule.

9. The method of claim 8, further comprising the step of:
contacting the terminal phosphate reactive linker modified biomolecule with a modifying agent to form biomolecule having a linker attached to its terminal phosphate group and a modifying agent attached to the reactive site of the linker,
where the modifying agent has a detectable property or is capable of interfering with, quenching, augmenting, reducing or enhancing a detectable property of an external biomolecule, biomolecular complex or biomolecular assembly.

10. The method of claim 9, wherein the nucleotides, phosphorylated polypeptides, phosphorylated proteins, phosphorylated sugars or sacchrides, phosphorylated carbohydrates, phosphorylated enzymes, phosphorylated membranes, phosphorylated cells, phosphorylated tissues, phospholipids or any other bio-material or organized structure bearing at least one phosphate group or mixtures or combinations thereof and wherein the linker is a compound having a formula Q-E-R-E′-Q′, where Q is a leaving group, E and E′ are B, C, Si, Ge, N, P, As, O, S, and/or Se atom-containing moieties, Q′ is a leaving group or a protecting or blocking group, and R is an alkenyl group, an arenyl group, an aralkenyl group and/or a alkarenyl group.

11. The method of claim 10, wherein the modifying agent comprises:
a molecule having one attachment site or a plurality of attachment sites, each site bearing a leaving group.

12. The method of claim 11, wherein the molecule comprises a molecular core and the site or sites extend out from the core.

13. The method of claim 11, wherein:
the biomolecule comprises a nucleotide comprising a nucleoside or nucleoside analog having at least one phosphate group attached at its 5′ hydroxy group, and
the core comprises (a) one quencher or a plurality of quenchers, or (b) an acceptor fluorophore or a plurality of acceptor fluorophore, or (c) an acceptor fluorophore or a plurality of acceptor fluorophore and a donor fluorophore or a plurality of donor fluorophore for the acceptor or acceptors.

14. The method of claim 11, wherein the core is selected from the group consisting of bi-functional molecules, polyfunctional molecules, polyfunctional boron-nitride nanostructures, polyfunctional carbon nanostructures, polyfunctional dendrimers, polyfunctional oligomers, polyfunctional polymers, polyfunctional metal oxide nanostructures, polyfunctional quantum dots, polyfunctional metal clusters, polyfunctional nanoshells, polyfunctional liposomes, or any other structure that can support attachment of a plurality of nucleotides through their terminal phosphate or mixtures of combinations thereof.

15. The method of claim 11, wherein the quenchers are the same or different, the acceptor fluorophores are the same or different or the acceptor fluorophores are the same or different and the donor fluorophores for the acceptors are the same of different.

16. A biomolecular composition comprising:
a molecular core,
a first plurality of attachment sites extending out from the core,
a second plurality of biomolecules, each biomolecule including a phosphate-containing group,
where each biomolecule is attached to an attachment site through a direct bond to a terminal phosphate moiety of the phosphate-containing group or through a linker interposed between the site and the terminal phosphate moiety of the phosphate-containing group.

17. The composition of claim 16, wherein:
the biomolecule comprises a nucleotide comprising a nucleoside or nucleoside analog having at least one phosphate group attached at its 5′ hydroxy group, and
the core includes: (a) one quencher or a plurality of quenchers, or (b) an acceptor fluorophore or a plurality of acceptor fluorophore, or (c) an acceptor fluorophore or a plurality of acceptor fluorophore and a donor fluorophore or a plurality of donor fluorophore for the acceptor or acceptors, where the quenchers are the same or different, the acceptor fluorophores are the same or different or the acceptor fluorophores are the same or different and the donor fluorophores for the acceptors are the same of different.

18. The composition of claim 16, wherein the core is selected from the group consisting of bi-functional molecules, polyfunctional molecules, polyfunctional boron-nitride nanostructures, polyfunctional carbon nanostructures, polyfunctional dendrimers, polyfunctional oligomers, polyfunctional polymers, polyfunctional metal oxide nanostructures, polyfunctional quantum dots, polyfunctional metal clusters, polyfunctional nanoshells, polyfunctional liposomes, or any other structure that can support attachment of a plurality of nucleotides through their terminal phosphate or mixtures of combinations thereof.

19. The composition of claim 16, wherein the biomolecules are the same or different.

20. The composition of claim 16, wherein the linkers are the same or different and are compounds having a formula Q-E-R-E′-Q′, where Q is a leaving group, E and E′ are B, C, Si, Ge, N, P, As, O, S, and/or Se atom-containing moieties, Q′ is a leaving group or a protecting or blocking group, and R is an alkenyl group, an arenyl group, an aralkenyl group and/or a alkarenyl group.

21. A method for sequencing comprising the steps of:
providing a biomolecular composition comprising:
a molecular core,
first plurality of attachment sites extending out from the core,
a second plurality of biomolecules, each biomolecule including a phosphate-containing group,
where each biomolecule is attached to an attachment site through a direct bond to the site an a terminal phosphate moiety of the phosphate-containing group or through a linker interposed between the site and the terminal phosphate moiety of the phosphate-containing group,
providing a sequencing solution including a polymerizing agent, a primer-template duplex, and sequencing buffer,
detecting a plurality of detectable events evidencing binding and/or nucleotide incorporation events, and
analyzing the events to determine a sequence of nucleotide incorporations complementary to a sequence on the template.

22. The method of claim 21, wherein:
the biomolecule comprises a nucleotide comprising a nucleoside or nucleoside analog having at least one phosphate group attached at its 5′ hydroxy group, and
the core includes: (a) one quencher or a plurality of quenchers, or (b) an acceptor fluorophore or a plurality of acceptor fluorophore; (c) an acceptor fluorophore or a plurality of acceptor fluorophore and a donor fluorophore or a plurality of donor fluorophore for the acceptor or acceptors, where the quenchers are the same or different, the acceptor fluorophores are the same or different or the acceptor fluorophores are the same or different and the donor fluorophores for the acceptors are the same of different.

23. The method of claim 21, wherein the core is selected from the group consisting of bi-functional molecules, polyfunctional molecules, polyfunctional boron-nitride nanostructures, polyfunctional carbon nanostructures, polyfunctional dendrimers, polyfunctional oligomers, polyfunctional polymers, polyfunctional metal oxide nanostructures, polyfunctional quantum dots, polyfunctional metal clusters, polyfunctional nanoshells, polyfunctional liposomes, or any other structure that can support attachment of a plurality of nucleotides through their terminal phosphate or mixtures of combinations thereof.

24. The method of claim 21, wherein the biomolecules are the same or different.

25. The composition of claim 21, wherein the linkers are the same or different and are compounds having a formula Q-E-R-E′-Q′, where Q is a leaving group, E and E′ are B, C, Si, Ge, N, P, As, O, S, and/or Se atom-containing moieties, Q′ is a leaving group or a protecting or blocking group, and R is an alkenyl group, an arenyl group, an aralkenyl group and/or a alkarenyl group.


SEQUENCE LISTING (1 sequence)

SEQ ID NO: 1 - DNA, 10 bp, Artificial Sequence
  synthetic construct
aaggagagaa